(19) United States
(12) Patent Application Publication
(10) Pub. No.: US 2023/0058961 A1
(43) Pub. Date: Feb. 23, 2023

(54) SUBSTITUTED HETEROCYCLICS WITH THERAPEUTIC ACTIVITY IN HIV

(71) Applicant: New York Blood Center, Inc., New York, NY (US)

(72) Inventors: Asim Kumar DEBNATH, Fort Lee, NJ (US); Francesca CURRELI, West New York, NJ (US)

(21) Appl. No.: 17/777,923

(22) PCT Filed: Nov. 19, 2020
(86) PCT No.: PCT/US2020/061239

§ 371 Date: May 18, 2022

Related U.S. Application Data
(60) Provisional application No. 62/937,665, filed on Nov. 19, 2019.

(51) Int. Cl. A61K 31/4025; A61K 31/426

(52) U.S. Cl. CPC A61K 31/4025; A61K 31/426

(57) ABSTRACT

Substituted heterocyclic substituted pyrrole carboxamide compounds such as those represented by Formula I or Formula II are provided herein. Such compounds, or pharmaceutically acceptable salts thereof, can be used in the treatment of HIV infection and related conditions.

### RELATED APPLICATIONS

This application claims the benefit of U.S. Provisional Patent Application No. 62/937,665, filed Nov. 19, 2019, which is incorporated herein by reference in its entirety.

### GOVERNMENT SUPPORT

This invention was made with government support under Grant Number R01AI104416 awarded by the National Institutes of Health. The Government has certain rights in the invention.

### BACKGROUND

Human immunodeficiency virus (HIV) is known to cause AIDS. Human immunodeficiency virus type 1 (HIV-1) cell entry process is thought to start when its surface envelope glycoproteins gp120 bind to the host cell primary receptor CD4. The binding triggers conformational changes in gp120 that facilitate its binding to the host cell co-receptor (secondary) CCR5 or CXCR4. There is no drug available yet that targets HIV-1 gp120.

### SUMMARY

Disclosed herein are anti-HIV compounds and methods of treating HIV infection utilizing them. Generally, the compounds are heterocyclic substituted pyrrole carboxamides.

In some embodiments, the anti-HIV compounds are represented by Formula (I):

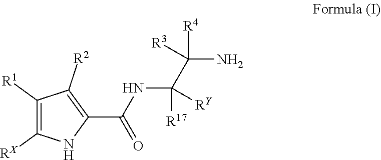

or a pharmaceutically acceptable salt thereof,
wherein in some embodiments:
each of R¹, R², R³, and R⁴, is, independently, H or C₁₋₃ alkyl;
R¹⁷ is H or CH₃;

### R^{X} is

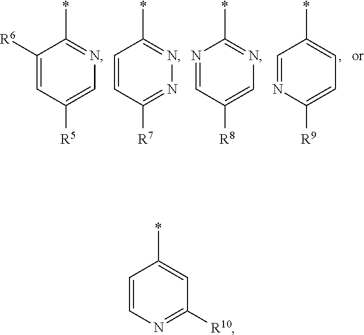

wherein * indicates the point of attachment to the pyrrolyl ring,
each of R⁵, R⁷, R⁸, and R⁹ is H, CH₃, halogen, or CF₃;
R⁶ is H or halogen; and
R¹⁰ is H, halogen, CF₃, OCH₃ or OCF₃; and

### R^{Y} is

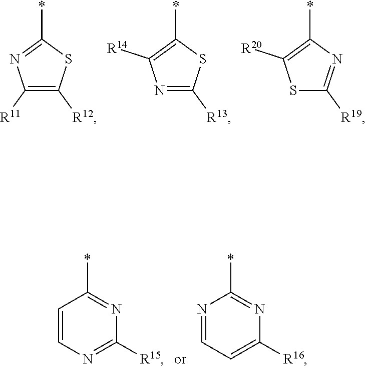

wherein * indicates the point of attachment to the Formula I backbone; and each of R¹¹, R¹², R¹³, R¹⁴, R¹⁵, R¹⁶, R¹⁹, and R²⁰, is, independently, H, CH₂OH, CH₂CH₂OH, (CH₂)₃OH or CH(OH)CH₂OH.

In some embodiments, the anti-HIV compounds are represented by Formula (II):

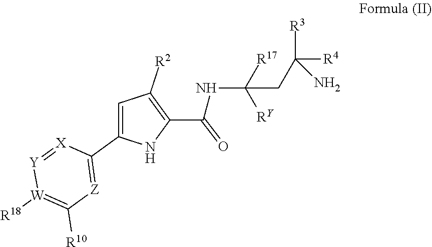

or a pharmaceutically acceptable salt thereof,
wherein in some embodiments:

### W is C or N;

X, Y, and Z are independently CH or N, and at least one of W, X, Y, or Z is N; if more than one of W, X, Y, or Z is N, then only X and Y, or X and Z, are both N.
each of R², R³, and R⁴, is, independently, H or CH₃;
R¹⁰ is H when X is N, and is H or OCH₃ when X is CH;

### R^{Y} is

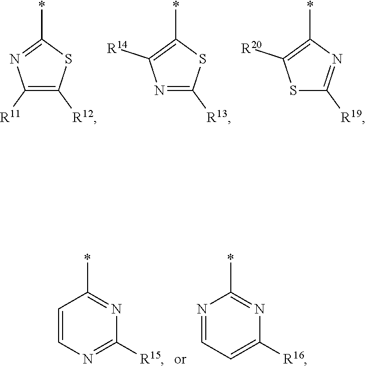

wherein * indicates the point of attachment to the Formula II backbone; and
each of R¹¹, R¹², R¹³, R¹⁴, R¹⁵, R¹⁶, R¹⁹, and R²⁰, is, independently, H, CH₂OH, CH₂CH₂OH, (CH₂)₃OH, or CH(OH)CH₂OH;
R¹⁷ is H or CH₃; and
R¹⁸ is absent when W is N, and is H, CH₃, Cl, F, CH₂F, CHF₂, or CF₃ when W is C.

Some embodiments include a pharmaceutical composition, such as an antiviral composition, comprising a compound represented by Formula (I), Formula (II), or species or subgenera thereof.

In some embodiments, the compound of Formula I or Formula II has a selectivity index (SI=CC₅₀/IC₅₀) greater than a threshold value, which in some embodiments is 100, 200, 300, or 400. Other embodiments exclude subject compounds with an SI less than a threshold value, which in some embodiments is 20, 30, 40, 50, 60, or 70.

In some embodiments, the compound of Formula I or Formula II has an IC₅₀ below a threshold value, which in some embodiments is 20, 15, 10, 5, 2, 1, 0.5, or 0.2 μM. Other embodiments exclude subject compounds with an IC₅₀ greater than a threshold value, which in some embodiments is 1, 2, 3, 5, 10, or 15 μM.

In some embodiments, the compound of Formula I or Formula II has a CC₅₀ greater than a threshold value, which in some embodiments is 60, 70, 80, 90, 100, 110, 120, 130, or 140 μM. Other embodiments exclude subject compounds with a CC₅₀ less than a threshold value, which in some embodiments is 70, 60, 50, 40, 30 μM.

Some embodiments, include a method of inhibiting HIV comprising administering a subject compound to a human being infected with HIV virus.

Some embodiments include a method of treating HIV infection comprising administering a subject compound to a human being infected with HIV virus.

Further embodiments, corresponding to methods of treatment, include use of a subject compound in the manufacture of a medicament for treatment of HIV infection, and the like, or the subject compounds for use in treating HIV infection, and the like.

Further embodiments include methods of preparing the subject compounds according to the synthetic schemes disclosed in Examples 5 and 6 (below).

### BRIEF DESCRIPTION OF THE DRAWINGS

FIG. 1 depicts the infectivity of CD4 negative Cf2Th-CCR5 cells by HIV-1_{NL4-3/ADA} (CD4-dependent virus) in the presence of indicated control and subject compounds.

### DETAILED DESCRIPTION

Disclosed herein are compounds useful for treating or preventing HIV infection, and methods of using those compounds. Some of the compounds described herein may target and inhibit gp120 from binding to the host cell receptor, CD4. In some embodiments, the herein disclosed compounds do not promote binding to CCR5 by gp120 nor thereby facilitate HIV entry into CD4 negative cells.

Unless otherwise indicated, when a compound or chemical structural feature such as aryl is referred to as being “optionally substituted,” it includes a feature that has no substituents (i.e. unsubstituted), or a feature that is “substituted,” meaning that the feature has one or more substituents. The term “substituent” includes a moiety that replaces one or more hydrogen atoms in a parent compound or structural feature. The term “replaces” is merely used herein for convenience, and does not require that the compound be formed by replacing one atom with another.

For convenience, the term “molecular weight” is used with respect to a moiety or part of a molecule to indicate the sum of the atomic masses of the atoms in the moiety or part of a molecule, even though it may not be a complete molecule. If a substituent is anionic or cationic, only the covalently bonded atoms are counted in the molecular weight.

Although counter-ions can be present, they are not included in the determination of molecular weight. Thus, —CO₂⁻Na⁺ would be considered have a molecular weight of about 44 Da and not about 67 Da.

As used herein, the term “alkyl” is a moiety composed of carbon and hydrogen containing no double or triple bonds. Alkyl may be linear alkyl, branched alkyl, or a combination thereof, and in some embodiments, may contain from one to thirty-five carbon atoms. In some embodiments, alkyl may include C₁₋₁₀ linear alkyl, for example C₁₋₃ or C₁₋₆ alkyl, such as methyl (—CH₃), ethyl (—CH₂CH₃), n-propyl (—CH₂CH₂CH₃), n-butyl (—CH₂CH₂CH₂CH₃), n-pentyl (—CH₂CH₂CH₂CH₂CH₃), n-hexyl (—CH₂CH₂CH₂CH₂CH₂CH₃), etc.; or C₃₋₆ branched alkyl, such as C₃H7 (e.g. isopropyl), C₄H9 (e.g. branched butyl isomers), C₅H11 (e.g. branched pentyl isomers), or C₆H13 (e.g. branched hexyl isomers). As used herein C₁₋₃ alkyl means a methyl (C₁), ethyl (C₂), or propyl group (C₃), or any combination thereof. Propyl may be n-propyl, isopropyl, cyclopropyl, or any subset thereof.

As used herein the term “aryl” is a ring or a ring system having at least one aromatic ring, such as phenyl, naphthyl, etc.

The term “heteroaryl” refers to an “aryl” that has one or more heteroatoms in the ring or ring system. Examples of “heteroaryl” may include, but are not limited to, pyridinyl, pyrimidinyl, pyridazinyl, furyl, thienyl, oxazolyl, thiazolyl, pyrrolyl, imidazolyl, indolyl, quinolinyl, benzofuranyl, benzothienyl, benzooxazolyl, benzothiazolyl, benzoimidazolyl, etc.

As used herein, the term “halogen” can include Cl, F, Br, or I. Some embodiments specifically include one or more of these species. In some embodiments, the halogen is limited to: Cl, F, or I; Cl or F; Cl; F; or I. Some embodiments specifically exclude one or more of these species.

The compounds provided herein may include pharmaceutically acceptable salts, such as sodium, potassium, and ammonium salts, as well as pharmaceutically acceptable salts found in Remington's Pharmaceutical Sciences, 17^{th} ed., Mack Publishing Company, Easton, Pa., 1985, p. 1418 and Journal of Pharmaceutical Science, 66, 2 (1977), each of which is incorporated herein by reference in its entirety. The compounds provided herein may also take the form of: prodrugs, such as ester prodrugs; alternate solid forms, such as polymorphs, solvates, hydrates, etc.; tautomers; or any other chemical species that may rapidly convert to a compound described herein under conditions in which the compounds are used as described herein.

In some embodiments, the herein disclosed compounds have a structure corresponding to Formula I

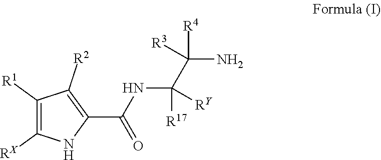

or a pharmaceutically acceptable salt thereof, wherein the R groups are as defined below.

In some embodiments, the herein disclosed compounds have a structure corresponding to Formula II

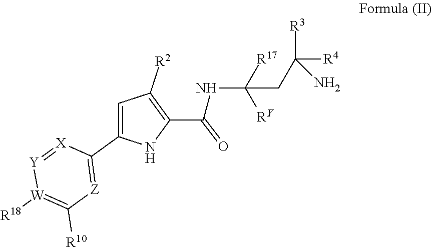

or a pharmaceutically acceptable salt thereof, wherein W, X, Y, Z, and the R groups are as defined below.

With respect to any relevant structural representation, in certain embodiments R¹ is, independently, H, or C₁₋₃ alkyl. In some embodiments, R¹ is H. In some embodiments, R¹ is methyl. In some embodiments, R¹ is ethyl. In some embodiments, R¹ is propyl.

Some of these embodiments specifically include one or more of these species. Some of these embodiments specifically exclude one or more of these species.

With respect to any relevant structural representation, in certain embodiments R² is, independently, H, or C₁₋₃ alkyl. In some embodiments, R² is H. In some embodiments, R² is methyl. In some embodiments, R² is ethyl. In some embodiments, R² is propyl.

Some of these embodiments specifically include one or more of these species. Some of these embodiments specifically exclude one or more of these species.

With respect to any relevant structural representation, in certain embodiments R³ is, independently, H, or C₁₋₃ alkyl. In some embodiments, R³ is H. In some embodiments, R³ is methyl. In some embodiments, R³ is ethyl. In some embodiments, R³ is propyl.

Some of these embodiments specifically include one or more of these species. Some of these embodiments specifically exclude one or more of these species.

With respect to any relevant structural representation, in certain embodiments R⁴ is, independently, H, or C₁₋₃ alkyl. In some embodiments, R⁴ is H. In some embodiments, R⁴ is methyl. In some embodiments, R⁴ is ethyl. In some embodiments, R⁴ is propyl. Some of these embodiments specifically include one or more of these species. Some of these embodiments specifically exclude one or more of these species.

With respect to any relevant structural representation, in certain embodiments R^{X} is

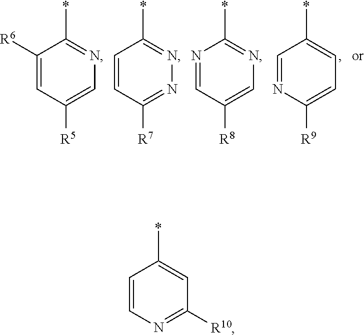

wherein * indicates the point of attachment to the pyrrolyl ring, as in Formula I. Some of these embodiments specifically include one or more of these species. Some of these embodiments specifically exclude one or more of these species.

With respect to any relevant structural representation, in certain embodiments R⁵ is independently, H, halogen, CH₃, or CF₃. In some embodiments, R⁵ is H. In some embodiments, R⁵ is halogen, or any subset thereof, as defined above. Some of these embodiments specifically include one or more of these species. Some of these embodiments specifically exclude one or more of these species.

With respect to any relevant structural representation, in certain embodiments R⁶ is, independently, H or halogen. In some embodiments, R⁶ is H. In some embodiments, R⁶ is halogen, or any subset thereof, as defined above. Some of these embodiments specifically include one or more of these species. Some of these embodiments specifically exclude one or more of these species.

With respect to any relevant structural representation, in certain embodiments R⁷ is, independently, H, halogen, CH₃, or CF₃. In some embodiments, R⁷ is H. In some embodiments, R⁷ is halogen, or any subset thereof, as defined above. Some of these embodiments specifically include one or more of these species. Some of these embodiments specifically exclude one or more of these species.

With respect to any relevant structural representation, in certain embodiments R⁸ is, independently, H, halogen, CH₃, or CF₃. In some embodiments, R⁸ is H. In some embodiments, R⁸ is halogen, or any subset thereof, as defined above. Some of these embodiments specifically include one or more of these species. Some of these embodiments specifically exclude one or more of these species.

With respect to any relevant structural representation, in certain embodiments R⁹ is, independently, H, halogen, CH₃, or CF₃. In some embodiments, R⁹ is H. In some embodiments, R⁹ is halogen, or any subset thereof, as defined above. Some of these embodiments specifically include one or more of these species. Some of these embodiments specifically exclude one or more of these species.

With respect to any relevant structural representation, in certain embodiments R¹⁰ is, independently, H, halogen, OCH₃, CF₃, or OCF₃. In some embodiments, R¹⁰ is H.

In some embodiments, R¹⁰ is halogen, or any subset thereof, as defined above. Some of these embodiments specifically include one or more of these species. Some of these embodiments specifically exclude one or more of these species.

With respect to any relevant structural representation, in certain embodiments R^{Y} is

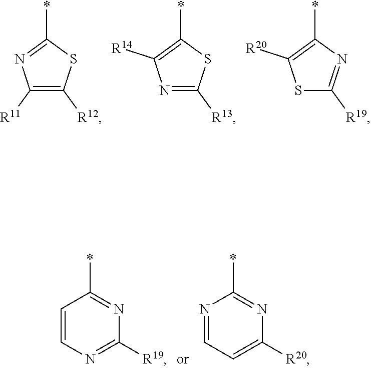

wherein * indicates the point of attachment to the Formula I or II backbone. Some of these embodiments specifically include one or more of these species. Some of these embodiments specifically exclude one or more of these species.

With respect to any relevant structural representation, in certain embodiments when R^{Y} is

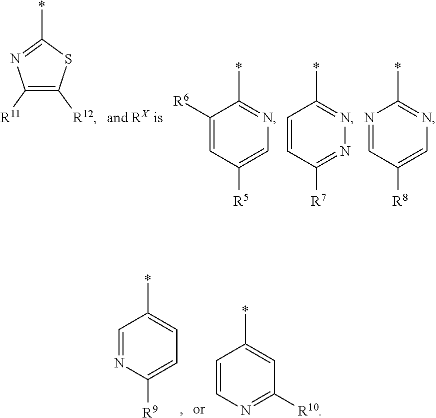

Some of these embodiments specifically include one or more of these species of R^{X}.
Some of these embodiments specifically exclude one or more of these species of R^{X}.

With respect to any relevant structural representation, in certain embodiments when R^{Y} is

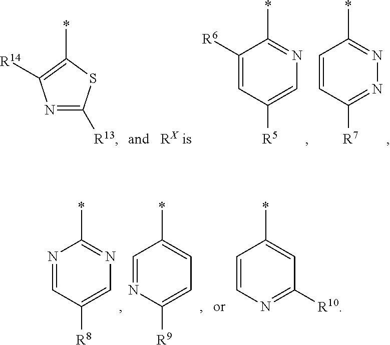

Some of these embodiments specifically include one or more of these species of R^{X}.
Some of these embodiments specifically exclude one or more of these species of R^{X}.

With respect to any relevant structural representation, in certain embodiments when R^{Y} is

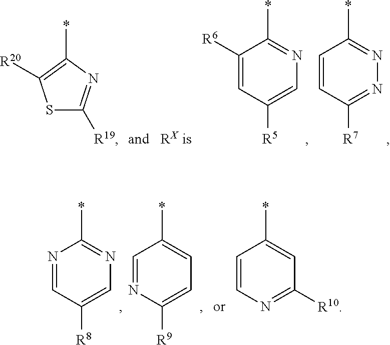

Some of these embodiments specifically include one or more of these species of R^{X}.
Some of these embodiments specifically exclude one or more of these species of R^{X}.

With respect to any relevant structural representation, in certain embodiments R¹¹ is, independently, H, CH₂OH, CH₂CH₂OH, (CH₂)₃OH, or CH(OH)CH₂OH. In some embodiments, R¹¹ is H. In some embodiments, R¹¹ is CH₂OH. Some of these embodiments specifically include one or more of these species. Some of these embodiments specifically exclude one or more of these species.

With respect to any relevant structural representation, in certain embodiments R¹² is, independently, H, CH₂OH, CH₂CH₂OH, (CH₂)₃OH, or CH(OH)CH₂OH. In some embodiments, R¹² is H. In some embodiments, R¹¹ is CH₂OH. Some of these embodiments specifically include one or more of these species. Some of these embodiments specifically exclude one or more of these species.

In some embodiments, R¹¹ is H when R¹² is H. In some embodiments, R¹¹ is H when R¹² is not H. In some embodiments, R¹¹ is H when R¹² is CH₂OH. In some embodiments, R¹¹ is CH₂OH when R¹² is H. In some embodiments, R¹¹ is H when R¹² is CH(OH)CH₂OH. In some embodiments, R¹¹ is CH(OH)CH₂OH when R¹² is H. In some embodiments, R¹¹ is CH₂OH when R¹² is CH₂OH. In some embodiments, R¹¹ is (CH₂)₂OH when R¹² is H. In some embodiments, R¹¹ is H when R¹² is (CH₂)₃OH. In some embodiments, R¹¹ is (CH₂)₂OH when R¹² is H. In some embodiments, R¹¹ H is when R¹² is (CH₂)₃OH. The additional pairings of the substituents of R¹¹ and R¹² constitute further embodiments.

With respect to any relevant structural representation, in certain embodiments R¹³ is, independently, H, CH₂OH, (CH₂)₂OH, (CH₂)₃OH or CH(OH)CH₂OH. In some embodiments, R¹³ is H. Some of these embodiments specifically include one or more of these species. Some of these embodiments specifically exclude one or more of these species.

With respect to any relevant structural representation, in certain embodiments R¹⁴ is, independently, H, CH₂OH, (CH₂)₂OH, (CH₂)₃OH or CH(OH)CH₂OH. In some embodiments, R¹⁴ is H. Some of these embodiments specifically include one or more of these species. Some of these embodiments specifically exclude one or more of these species.

In some embodiments, R¹³ is H when R¹⁴ is H. In some embodiments, R¹³ is H when R¹⁴ is not H. In some embodiments, R¹³ is H when R¹⁴ is CH₂OH. In some embodiments, R¹³ is CH₂OH when R¹⁴ is H. In some embodiments, R¹³ is H when R¹⁴ is CH(OH)CH₂OH. In some embodiments, R¹³ is CH(OH)CH₂OH when R¹⁴ is H. In some embodiments, R¹³ is CH₂OH when R¹⁴ is CH₂OH. In some embodiments, R¹³ is (CH₂)₂OH when R¹⁴ is H. In some embodiments, R¹³ is H when R¹⁴ is (CH₂)₃OH. In some embodiments, R¹³ is (CH₂)₂OH when R¹⁴ is H. In some embodiments, R¹³ H is when R¹⁴ is (CH₂)₃OH. The additional pairings of the substituents of R¹³ and R¹⁴ constitute further embodiments.

With respect to any relevant structural representation, in certain embodiments R¹⁵ is, independently, H, CH₂OH, (CH₂)₂OH, (CH₂)₃OH or CH(OH)CH₂OH. In some embodiments, R¹⁵ is H. Some of these embodiments specifically include one or more of these species. Some of these embodiments specifically exclude one or more of these species.

With respect to any relevant structural representation, in certain embodiments R¹⁶ is, independently, H, CH₂OH, (CH₂)₂OH, (CH₂)₃OH or CH(OH)CH₂OH. In some embodiments, R¹⁶ is H. Some of these embodiments specifically include one or more of these species. Some of these embodiments specifically exclude one or more of these species.

With respect to any relevant structural representation, in certain embodiments R¹⁷ is, independently, H or CH₃. In some embodiments, R¹⁷ is H. In some embodiments, R¹⁷ is CH₃. Some of these embodiments specifically include one or more of these species. Some of these embodiments specifically exclude one or more of these species.

With respect to any relevant structural representation, such as Formula II, in certain embodiments W is C or N. In some embodiments, W is C. In some embodiments, W is N. Some of these embodiments specifically include one or more of these species. Some of these embodiments specifically exclude one or more of these species.

With respect to any relevant structural representation, such as Formula II, in certain embodiments each of X, Y, and Z is, independently, CH or N, except that among W, X, Y, and Z, only X and Y, or X and Z, are both N in any compound. Thus, with respect to W, X, Y, and Z, in some embodiments: only W is N; only X is N; only Y is N; X and Y are N; or X and Z are N. In some embodiments, Z is N only when X is N. Some of these embodiments specifically include one or more of these species or combinations. Some of these embodiments specifically exclude one or more of these species or combinations.

With respect to any relevant structural representation, such as Formula II, in certain embodiments R¹⁸ is, independently, H, CH₃, Cl, F, CH₂F, CHF₂, or CF₃ when W is C; or absent when W is N. Some of these embodiments specifically include one or more of these species. Some of these embodiments specifically exclude one or more of these species. In some of these embodiments, R¹⁸ is not H.

With respect to any relevant structural representation, in certain embodiments R¹⁹ is, independently, H, CH₂OH, (CH₂)₂OH, (CH₂)₃OH or CH(OH)CH₂OH. In some embodiments, R¹⁹ is H. Some of these embodiments specifically include one or more of these species. Some of these embodiments specifically exclude one or more of these species.

With respect to any relevant structural representation, in certain embodiments R²⁰ is, independently, H, CH₂OH, (CH₂)₂OH, (CH₂)₃OH or CH(OH)CH₂OH. In some embodiments, R²⁰ is H. Some of these embodiments specifically include one or more of these species. Some of these embodiments specifically exclude one or more of these species.

In some embodiments, R¹⁹ is H when R²⁰ is H. In some embodiments, R¹⁹ is H when R²⁰ is not H. In some embodiments, R¹⁹ is H when R²⁰ is CH₂OH. In some embodiments, R¹⁹ is CH₂OH when R²⁰ is H. In some embodiments, R¹⁹ is H when R²⁰ is CH(OH)CH₂OH. In some embodiments, R¹⁹ is CH(OH)CH₂OH when R²⁰ is H. In some embodiments, R¹⁹ is CH₂OH when R²⁰ is CH₂OH. In some embodiments, R¹⁹ is (CH₂)₂OH when R¹⁴ is H. In some embodiments, R¹⁹ is H when R²⁰ is (CH₂)₃OH. In some embodiments, R¹⁹ is (CH₂)₂OH when R²⁰ is H. In some embodiments, R¹⁹ H is when R²⁰ is (CH₂)₃OH. The additional pairings of the substituents of R¹⁹ and R²⁰ constitute further embodiments.

In some embodiments, a compound of Formula I or Formula II can be:

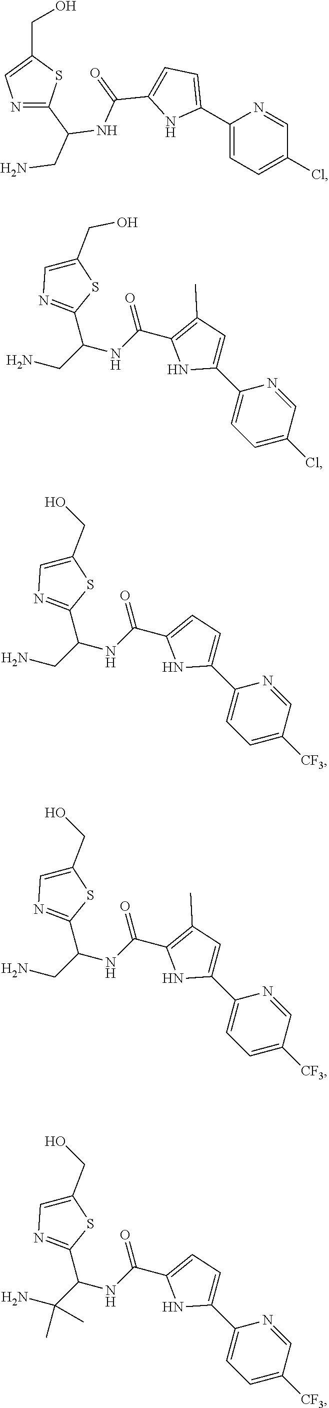 [SMILES: NCC(NC(=O)c1ccc(-c2ccc(Cl)cn2)[nH]1)c1ncc(CO)s1 ; Cc1cc(-c2ccc(Cl)cn2)[nH]c1C(=O)NC(CN)c1ncc(CO)s1 ; NCC(NC(=O)c1ccc(-c2ccc(C(F)(F)F)cn2)[nH]1)c1ncc(CO)s1 ; Cc1cc(-c2ccc(C(F)(F)F)cn2)[nH]c1C(=O)NC(CN)c1ncc(CO)s1 ; CC(C)(N)C(NC(=O)c1ccc(-c2ccc(C(F)(F)F)cn2)[nH]1)c1ncc(CO)s1]

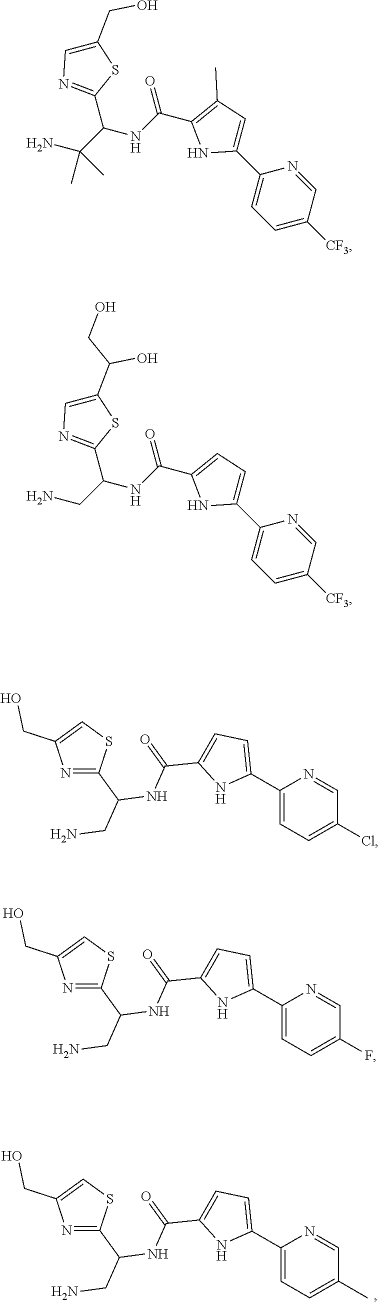 [SMILES: Cc1cc(-c2ccc(C(F)(F)F)cn2)[nH]c1C(=O)NC(c1ncc(CO)s1)C(C)(C)N ; NCC(NC(=O)c1ccc(-c2ccc(C(F)(F)F)cn2)[nH]1)c1ncc(C(O)CO)s1 ; NCC(NC(=O)c1ccc(-c2ccc(Cl)cn2)[nH]1)c1nc(CO)cs1 ; NCC(NC(=O)c1ccc(-c2ccc(F)cn2)[nH]1)c1nc(CO)cs1 ; Cc1ccc(-c2ccc(C(=O)NC(CN)c3nc(CO)cs3)[nH]2)nc1]

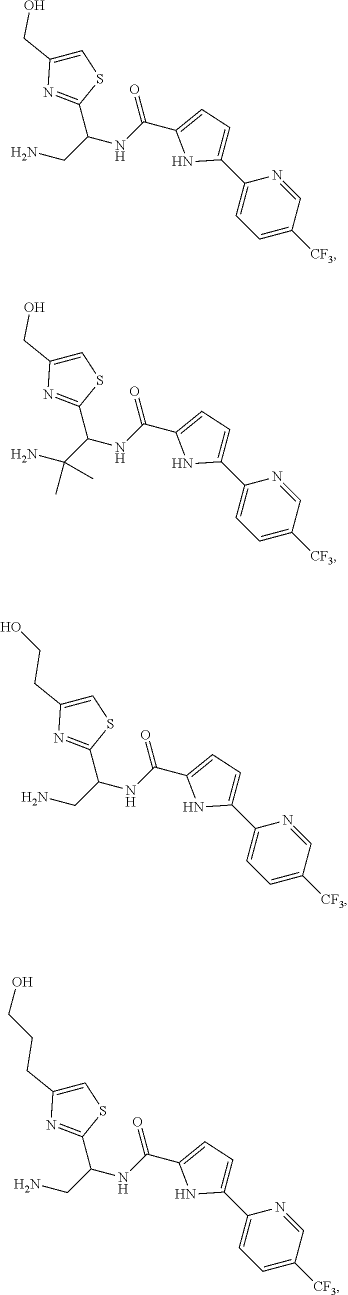 [SMILES: NCC(NC(=O)c1ccc(-c2ccc(C(F)(F)F)cn2)[nH]1)c1nc(CO)cs1 ; CC(C)(N)C(NC(=O)c1ccc(-c2ccc(C(F)(F)F)cn2)[nH]1)c1nc(CO)cs1 ; NCC(NC(=O)c1ccc(-c2ccc(C(F)(F)F)cn2)[nH]1)c1nc(CCO)cs1 ; NCC(NC(=O)c1ccc(-c2ccc(C(F)(F)F)cn2)[nH]1)c1nc(CCCO)cs1]

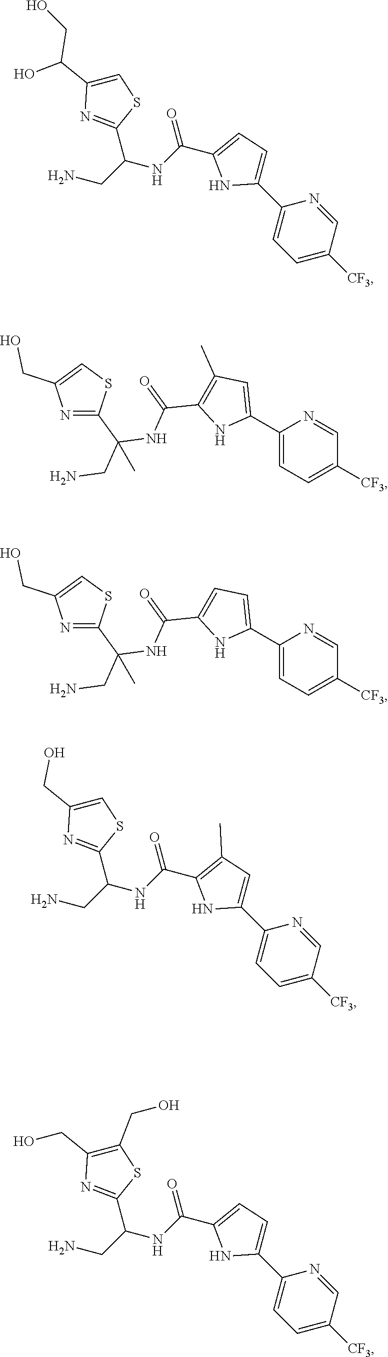 [SMILES: NCC(NC(=O)c1ccc(-c2ccc(C(F)(F)F)cn2)[nH]1)c1nc(C(O)CO)cs1 ; Cc1cc(-c2ccc(C(F)(F)F)cn2)[nH]c1C(=O)NC(C)(CN)c1nc(CO)cs1 ; CC(CN)(NC(=O)c1ccc(-c2ccc(C(F)(F)F)cn2)[nH]1)c1nc(CO)cs1 ; Cc1cc(-c2ccc(C(F)(F)F)cn2)[nH]c1C(=O)NC(CN)c1nc(CO)cs1 ; NCC(NC(=O)c1ccc(-c2ccc(C(F)(F)F)cn2)[nH]1)c1nc(CO)c(CO)s1]

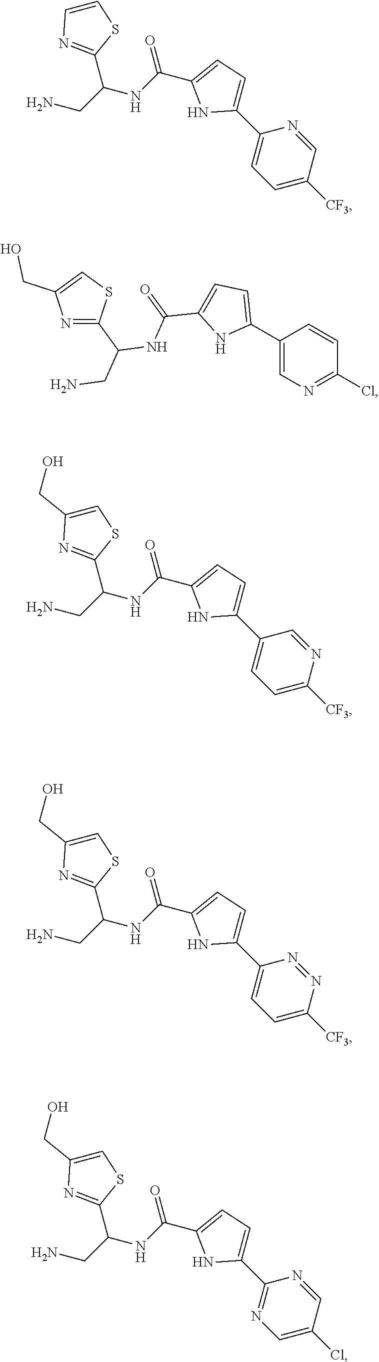 [SMILES: NCC(NC(=O)c1ccc(-c2ccc(C(F)(F)F)cn2)[nH]1)c1nccs1 ; NCC(NC(=O)c1ccc(-c2ccc(Cl)nc2)[nH]1)c1nc(CO)cs1 ; NCC(NC(=O)c1ccc(-c2ccc(C(F)(F)F)nc2)[nH]1)c1nc(CO)cs1 ; NCC(NC(=O)c1ccc(-c2ccc(C(F)(F)F)nn2)[nH]1)c1nc(CO)cs1 ; NCC(NC(=O)c1ccc(-c2ncc(Cl)cn2)[nH]1)c1nc(CO)cs1]

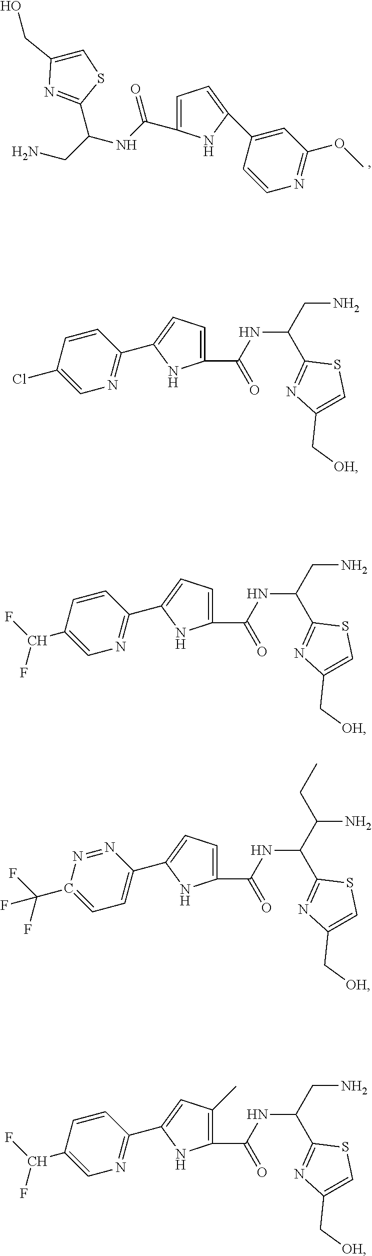

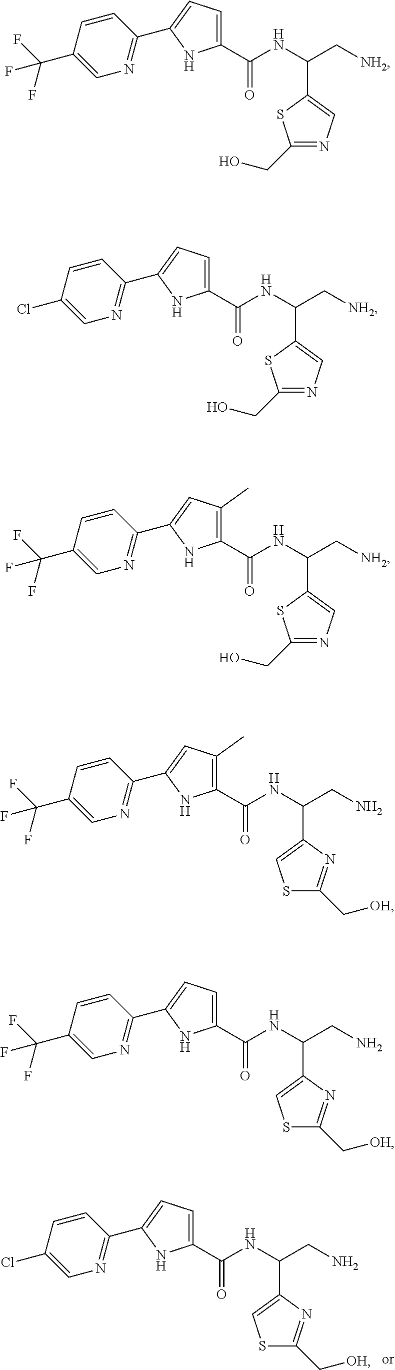 [SMILES: NCC(NC(=O)c1ccc(-c2ccc(C(F)(F)F)cn2)[nH]1)c1cnc(CO)s1 ; NCC(NC(=O)c1ccc(-c2ccc(Cl)cn2)[nH]1)c1cnc(CO)s1 ; Cc1cc(-c2ccc(C(F)(F)F)cn2)[nH]c1C(=O)NC(CN)c1cnc(CO)s1 ; Cc1cc(-c2ccc(C(F)(F)F)cn2)[nH]c1C(=O)NC(CN)c1csc(CO)n1 ; NCC(NC(=O)c1ccc(-c2ccc(C(F)(F)F)cn2)[nH]1)c1csc(CO)n1 ; NCC(NC(=O)c1ccc(-c2ccc(Cl)cn2)[nH]1)c1csc(CO)n1]

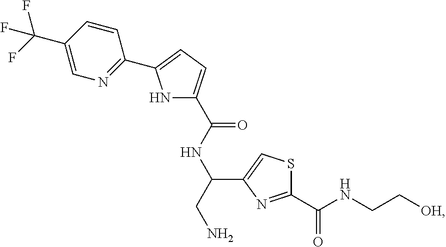 [SMILES: NCC(NC(=O)c1ccc(-c2ccc(C(F)(F)F)cn2)[nH]1)c1csc(C(=O)NCCO)n1]

or a pharmaceutically acceptable salt thereof. With respect to each of the above structures, in some embodiments, the compound is the R enantiomer, in some embodiments, the compound is the S enantiomer, in some embodiments, the compound is a mixture of R and S enantiomers (that is, racemic), and in some embodiments, the stereochemistry is unspecified. Each subset of these compounds constitutes a distinct embodiment. Some embodiments specifically include one or more of these species. Some embodiments specifically exclude one or more of these species.

In some embodiments, a compound of Formula I or Formula II is at least 90, 95, 97, 98, 99, 99.9, or 99.99 percent the S enantiomer of the compound. In some embodiments, a compound of Formula I or Formula II is at least 90, 95, 97, 98, 99, 99.9, or 99.99 percent the R enantiomer of the compound.

The compounds described herein, such as compounds of Formulae I and II (referred to hereafter as “subject compounds” or “subject compound”) may be used as inhibitors of human immunodeficiency virus (HIV) or for treating diseases, disorders, and conditions associated with HIV. Subject compounds may also be referred to as (heterocyclic) means for inhibiting HIV, means for inhibiting HIV entry, and the like. A pharmaceutical composition comprising at least one subject compound may be administered to individuals suffering from or susceptible to HIV-1 infection. In further embodiments the subject compounds are administered to a subject in need thereof, in methods of eradicating, reducing, or slowing an HIV infection, reducing viral load associated with HIV infection, reducing recurrence of HIV infection, of reducing an adverse physiological impact of an HIV infection, of inducing remission of an organ injury from an HIV infection, of reducing the physiological impact of long-term antiviral therapy for HIV infection, of prophylactically treating an HIV infection in a subject afflicted with a latent HIV infection. In an aspect of these embodiments the HIV infection is an HIV-1 infection.

The term “treating” or “treatment” broadly includes any kind of treatment activity, including the diagnosis, mitigation, or prevention of disease in human or other animals, or any activity that otherwise affects the structure or any function of the body of a human or other animals. Treatment activity includes the administration of the medicaments, dosage forms, and pharmaceutical compositions described herein to a patient, especially according to the various methods of treatment disclosed herein, whether by a healthcare professional, the patient him/herself, or any other person. Treatment activities include the orders, instructions, and advice of healthcare professionals such as physicians, physician's assistants, nurse practitioners, and the like, that are then acted upon by any other person including other healthcare professionals or the patient him/herself. In some embodiments, treatment activity can also include encouraging, inducing, or mandating that a particular medicament, or combination thereof, be chosen for treatment of a condition—and the medicament is actually used and benefit received thereby—by approving insurance coverage for the medicament, denying coverage for an alternative medicament, including the medicament on, or excluding an alternative medicament, from a drug formulary, or offering a financial incentive to use the medicament, as might be done by an insurance company or a pharmacy benefits management company, and the like. In some embodiments, treatment activity can also include encouraging, inducing, or mandating that a particular medicament be chosen for treatment of a condition—and the medicament is actually used and benefit received thereby—by a policy or practice standard as might be established by a hospital, clinic, health maintenance organization, medical practice or physicians group, and the like.

As used herein, the term “inhibiting HIV” means reducing the amount of virus produced (including completely blocking production of virus). Accordingly, inhibiting HIV may include preventing or reducing initial infection or transmission from cell to cell, or production of virus in or release of virus from an infected cell.

Appropriate excipients for use in a pharmaceutical composition comprising a subject compound (referred to hereafter as “subject compositions” or “subject composition”) may include, for example, one or more carriers, binders, fillers, vehicles, disintegrants, surfactants, dispersion or suspension aids, thickening or emulsifying agents, isotonic agents, preservatives, lubricants, and the like or combinations thereof, as suited to a particular dosage from desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. This document is incorporated herein by reference in its entirety.

As used herein, “pharmaceutically acceptable” indicates that the reagent or composition is not, or does not contain an agent or contaminant, respectively, that would preclude its use as a pharmaceutical product according to its intended use in the expected patient population. As HIV infection is primarily a human disease, (though in some embodiments treatment of other animals, for example, non-human primates, is not excluded) the standards of the US Food and Drug Administration, or corresponding authorities in other jurisdictions, for antiviral drugs would be of relevance in assessing pharmaceutical acceptability in preferred embodiments. Such considerations would include the absence of acute toxic effects, especially unrelated to the mechanism of action, or at least that the toxicity was minor or negligible in relation to the therapeutic benefit; non-exposure to reagents potentially contaminated with adventitious infectious agents; absence of oncogenic agents; and being aseptic or sterile.

A subject composition may be formulated for any desirable route of delivery including, but not limited to, parenteral, intravenous, intradermal, subcutaneous, oral, inhalative, transdermal, topical, transmucosal, rectal, intracisternal, intravaginal, intraperitoneal, buccal, and intraocular.

In certain aspects, parenteral, intradermal or subcutaneous formulations may be sterile injectable aqueous or oleaginous suspensions. Acceptable vehicles, solutions, suspensions and solvents may include, but are not limited to, water or other sterile diluent; saline; Ringer's solution; sodium chloride; fixed oils such as mono- or diglycerides; fatty acids such as oleic acid; polyethylene glycols; glycerine; propylene glycol or other synthetic solvents; antibacterial agents such as benzyl alcohol; antioxidants such as ascorbic acid; chelating agents such as ethylenediaminetetraacetic acid; buffers such as acetates, citrates or phosphates; and agents for the adjustment of tonicity such as sodium chloride or dextrose.

Solutions or suspensions used for parenteral, intradermal, or subcutaneous application may include one or more of the following components: a sterile diluent such as water for injection, saline solution, fixed oils, polyethylene glycols, glycerine; propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. The pH can be adjusted with acids or bases, such as hydrochloric acid or sodium hydroxide. The parenteral preparation may be enclosed in ampoules, disposable syringes or multiple dose vials made of glass or plastic.

Pharmaceutical compositions suitable for injectable use may include sterile aqueous solutions or dispersions and sterile powders for the extemporaneous preparation of sterile injectable solutions or dispersion. For intravenous administration, suitable carriers include, but are not limited to, saline, bacteriostatic water, CREMOPHOR EL (BASF, Parsippany, N.J.) or phosphate buffered saline (PBS). The solvent or dispersion medium may contain, for example, water, ethanol, polyol (for example, glycerol, propylene glycol, and liquid polyethylene glycol, and the like), and suitable mixtures thereof. Proper fluidity can be maintained, for example, by the use of a coating such as lecithin, by the maintenance of the required particle size in the case of dispersion and by the use of surfactants. Preventing growth of microorganisms can be achieved by various antibacterial and antifungal agents, for example, parabens, chlorobutanol, phenol, ascorbic acid, thimerosal, and the like. The composition may also include isotonic agents such as, for example, sugars; polyalcohols such as manitol; sorbitol; or sodium chloride. Prolonged absorption of injectable compositions can be enhanced by addition of an agent that delays absorption, such as, for example, aluminum monostearate or gelatin.

Oral compositions may include an inert diluent or an edible carrier. They may be enclosed in gelatin capsules or compressed into tablets. Tablets, pills, capsules, troches and the like can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

In addition to oral or injected administration, systemic administration may be by transmucosal or transdermal means. For transmucosal or transdermal administration, penetrants may be used. Such penetrants are generally known in the art, and include, for example, detergents, bile salts, and fusidic acid derivatives. Transdermal administration may include a bioactive agent and may be formulated into ointments, salves, gels, or creams as generally known in the art. Transmucosal administration may be accomplished through the use of nasal sprays or suppositories.

A subject compound may be administered in a therapeutically effective amount, according to an appropriate dosing regimen. As understood by a skilled artisan, an exact amount required may vary from subject to subject, depending on a subject's species, age and general condition, the severity of the infection, the particular agent(s) and the mode of administration. In some embodiments, about 0.001 mg/kg to about 50 mg/kg, of the subject compound (potentially in a pharmaceutical composition) based on the subject's body weight, is administered one or more times a day, to obtain the desired therapeutic effect. In other embodiments, about 0.01 mg/kg to about 25 mg/kg, of the subject compound, based on the subject's body weight, is administered one or more times a day, to obtain the desired therapeutic effect.

A total daily dosage of a subject compound can be determined by the attending physician within the scope of sound medical judgment. A specific therapeutically effective dose level for any particular patient or subject will depend upon a variety of factors including the disorder being treated and the severity of the disorder; the activity of the specific compound employed; the specific composition employed; the age, body weight, general health, sex and diet of the patient or subject; the time of administration, route of administration, and rate of excretion of the specific compound employed; the duration of the treatment; drugs used in combination or coincidental with the specific compound employed; and other factors well known in the medical arts.

In some embodiments, the methods of treating HIV infection provided herein comprise administering a compound of any one of Formulae I or II, or a pharmaceutically acceptable salt thereof, to a human being infected with HIV.

In some embodiments, the method of treating HIV infection provided herein comprise administering any one of compounds 1-70 (see Table 1), or a pharmaceutically acceptable salt thereof, to a human being infected with HIV. In some embodiments, one or more particular subject compounds are specifically included. For example some embodiments may specifically include compound 8 or 33 or both. In some embodiments, one or more particular subject compounds are specifically excluded. For example some embodiments may specifically exclude compounds 39-48, or any subset thereof.

### EXAMPLES

### Example 1. Antiviral Screening

The HIV-1 inhibitory activity and cytotoxicity of the subject compounds was evaluated. The anti-HIV-1 activity was assessed in a single-cycle assay by infecting TZM-bl indicator cells with the pseudovirus HIV-1 HXB2 according to established procedures (F. Curreli, Y. D. Kwon, D. S. Belov, R. R. Ramesh, A. V. Kurkin, A. Altieri, P. D. Kwong, A. K. Debnath, J Med Chem 2017, 60, 3124-3153; which is incorporated herein by reference for all that it teaches about the conduct and interpretation of the assay). Results with respect to anti-HIV activity and cytotoxicity are expressed as IC₅₀, CC₅₀, and selectivity index (SI=CC₅₀/IC₅₀) and presented in Table 1 for exemplary compounds according to Formulae I and II.

All of the compounds in Table 1 exhibit significant inhibitory activity against HIV. Of these compounds only Compounds 17-18, 20, 36, and 39-48 had an IC₅₀ exceeding 10 μM, and none exceeded 16 μM. Compounds 1, 5, 7-12, 21-22, 24, 26, 28, and 33 had an IC₅₀ less than 1 μM. Of these compounds only Compounds 9, 11-12, and 23-24 had a CC₅₀ less than 50 μM. Compounds 1-2, 13-20, 27, 29-30, 39-42, and 47-78 had a CC₅₀ greater than 100 μM. Of these compounds only Compounds 17-20, 36, 39-48 had an SI of less than 20. Compounds 1, 7-10, 21-22, and 33 had an SI greater than 100.

Comparator A

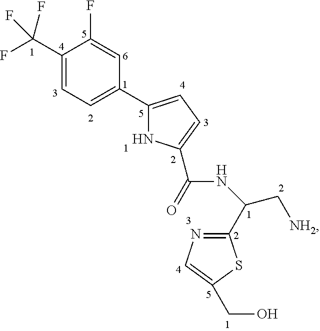 [SMILES: NCC(NC(=O)c1ccc(-c2ccc(C(F)(F)F)c(F)c2)[nH]1)c1ncc(CO)s1]

has an IC₅₀=0.089, CC₅₀=21.9 and SI=246; and

Comparator B

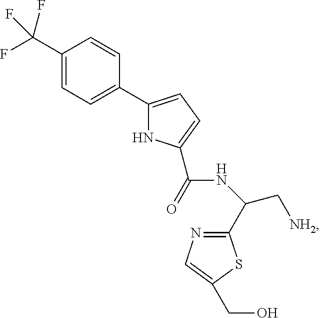 [SMILES: NCC(NC(=O)c1ccc(-c2ccc(C(F)(F)F)cc2)[nH]1)c1ncc(CO)s1]

has an IC₅₀=0.27, CC₅₀=34 and SI=126. For the majority of the 48 compounds exemplifying Formula II in Table 1, the 6-member ring is a pyridinyl ring as compared to a phenyl ring as found in Comparators A and B. This was observed to considerably improve cytotoxicity, irrespective of the position of “N” in the pyridinyl ring. Antiviral activity was dependent on other substituents on the 6-member and pyrrole rings, for example, a CH₃ group at R² improved antiviral activity, but it had no detrimental effect on the toxicity.

An electron-withdrawing substituent at position R¹⁸ mostly improved antiviral potency or retained the activity; however, the antiviral activity considerably dropped when introducing an electron-donating substituent at that position, such as CH₃, (Compound 19-20). Although fluorine is an electron-withdrawing substituent, its presence at R¹⁸ lowered the activity, most likely due to its small size. It is to be noted that when the “N” atom in pyridine is at position Y or W (see the structure in Table 1), the antiviral potency dropped dramatically. Similarly, a bulkier group at R¹⁰ also had detrimental effect on antiviral activity, which is most likely due to the steric limitation in the narrow hydrophobic cavity.

Substitutions on the thiazole ring and in the vicinity of the terminal amine typically had a greater effect on toxicity than antiviral activity. Introduction of CH₂OH or its higher congeners in position R¹¹ (Compounds 15-34) generally improved the cytotoxicity, but the introduction of a branched alcohol group (CHOHCH₂OH) at R¹¹, and separately at R¹², resulted in loss of activity (Compounds 29-30 and 13-14, respectively). The cytotoxicity of these compounds remains low (higher CC₅₀ values). Longer alcohol group, such as (CH₂)₂OH (Compounds 25-26) and (CH₂)₃OH (Compounds 27-28) were introduced and it was observed that the antiviral activity did not drop substantially. However, cytotoxicity was somewhat higher in a few cases, such as introduction of CH₃ group in position R³ and R⁴, which generally retained the antiviral potency, but the toxicity of those compounds was higher (Compounds 9-12 and 23-24).

TABLE 1
Anti-HIV-1 activity (IC₅₀) and cytotoxicity (CC₅₀) of subject compounds in single-cycle (TZM-bl cells) assay

| | |
|---|---|
| | Formula II |
| 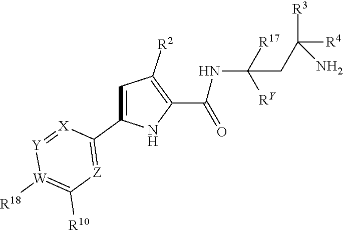 | |

| | | | | | | | | | | | | | | | μM | | |
|---|---|---|---|---|---|---|---|---|---|---|---|---|---|---|---|---|---|
| No. | Enantiomer | R^{Y} | R² | R³ | R⁴ | R¹⁰ | R¹¹ | R¹² | R¹⁷ | R¹⁸ | W | X | Y | Z | IC₅₀ | CC₅₀ | SI |
| 1 | fR | 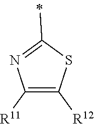 | H | H | H | H | H | CH₂OH | H | Cl | C | N | CH | CH | 0.85 ± 0.1 | 144 ± 7.5 | 169 |
| 2 | fS | 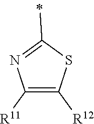 | H | H | H | H | H | CH₂OH | H | Cl | C | N | CH | CH | 2.7 ± 0.7 | 142 ± 1.7 | 52 |
| 3 | fR | 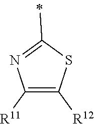 | CH₃ | H | H | H | H | CH₂OH | H | Cl | C | N | CH | CH | 1.2 ± 0.1 | 98.3 ± 4 | 81.9 |
| 4 | fS | 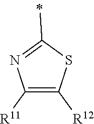 | CH₃ | H | H | H | H | CH₂OH | H | Cl | C | N | CH | CH | 2.4 ± 0.2 | 95 ± 3.6 | 39.6 |
| 5 | fR | 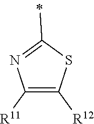 | H | H | H | H | H | CH₂OH | H | CF₃ | C | N | CH | CH | 0.96 | >122 | >127 |
| 6 | fS | 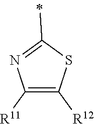 | H | H | H | H | H | CH₂OH | H | CF₃ | C | N | CH | CH | 1.2 | >122 | 101 |
| 7 | fR | 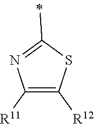 | CH₃ | H | H | H | H | CH₂OH | H | CF₃ | C | N | CH | CH | 0.36 ± 0.01 | 92.8 ± 2.4 | 258 |
| 8 | fS | 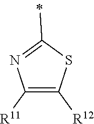 | CH₃ | H | H | H | H | CH₂OH | H | CF₃ | C | N | CH | CH | 0.16 ± 0.004 | 109.3 ± 2 | 683 |
| 9 | fR | 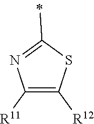 | H | CH₃ | CH₃ | H | H | CH₂OH | H | CF₃ | C | N | CH | CH | 0.187 | 74 ± 3.6 | 396 |
| 10 | fS | 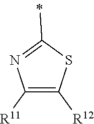 | H | CH₃ | CH₃ | H | H | CH₂OH | H | CF₃ | C | N | CH | CH | 0.5 | 41 ± 2.5 | 82 |
| 11 | fR | 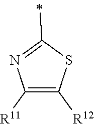 | CH₃ | CH₃ | CH₃ | H | H | CH₂OH | H | CF₃ | C | N | CH | CH | 0.6 ± 0.2 | 35.3 ± 2 | 59 |
| 12 | fS | 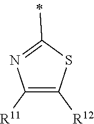 | CH₃ | CH₃ | CH₃ | H | H | CH₂OH | H | CF₃ | C | N | CH | CH | 0.43 ± 0.05 | 37.5 ± 2.2 | 87 |
| 13 | fR | 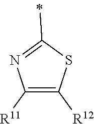 | CH₃ | H | H | H | H | CHOHCH₂OH | H | CF₃ | C | N | CH | CH | 2.1 ± 0.3 | >113 | >54 |
| 14 | fS | 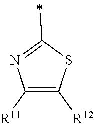 | CH₃ | H | H | H | H | CHOHCH₂OH | H | CF₃ | C | N | CH | CH | 5.9 ± 0.6 | >113 | >19 |
| 15 | fR | 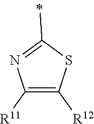 | H | H | H | H | CH₂OH | H | H | Cl | C | N | CH | CH | 5 ± 0.5 | >132 | >26 |
| 16 | fS | 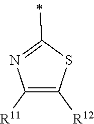 | H | H | H | H | CH₂OH | H | H | Cl | C | N | CH | CH | 1.7 ± 0.3 | >132 | >78 |
| 17 | fR | 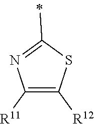 | H | H | H | H | CH₂OH | H | H | F | C | N | CH | CH | >11 | >138 | −12 |
| 18 | fS | 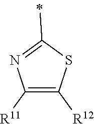 | H | H | H | H | CH₂OH | H | H | F | C | N | CH | CH | >11 | >138 | −12 |
| 19 | fR | 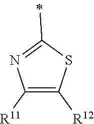 | H | H | H | H | CH₂OH | H | H | CH₃ | C | N | CH | CH | 9.6 ± 1.2 | >140 | >14 |
| 20 | fS | 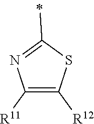 | H | H | H | H | CH₂OH | H | H | CH₃ | C | N | CH | CH | >11 | >140 | −13 |
| 21 | fR | 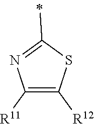 | H | H | H | H | CH₂OH | H | H | CF₃ | C | N | CH | CH | 0.5 | 96 ± 6 | 192 |
| 22 | fS | 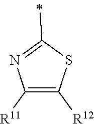 | H | H | H | H | CH₂OH | H | H | CF₃ | C | N | CH | CH | 0.3 | 124 ± 7 | 413 |
| 23 | fR | 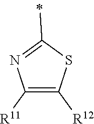 | H | CH₃ | CH₃ | H | CH₂OH | H | H | CF₃ | C | N | CH | CH | 1.73 | 61 ± 6.5 | 35 |
| 24 | fS | 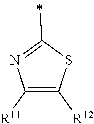 | H | CH₃ | CH₃ | H | CH₂OH | H | H | CF₃ | C | N | CH | CH | 0.58 | 59 ± 10 | 102 |
| 25 | fR | 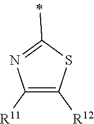 | H | H | H | H | (CH₂)₂OH | H | H | CF₃ | C | N | CH | CH | 1.7 | 100 ± 7 | 59 |
| 26 | fS | 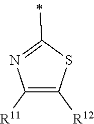 | H | H | H | H | (CH₂)₂OH | H | H | CF₃ | C | N | CH | CH | 0.76 | 91 ± 3.6 | 120 |
| 27 | fR | 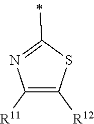 | H | H | H | H | (CH₂)₃OH | H | H | CF₃ | C | N | CH | CH | 2.3 ± 0.3 | >114 | >49.5 |
| 28 | fS | 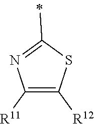 | H | H | H | H | (CH₂)₃OH | H | H | CF₃ | C | N | CH | CH | 0.89 ± 0.12 | 81.2 ± 1.3 | 91 |
| 29 | fR | 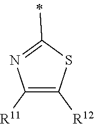 | H | H | H | H | CHOHCH₂OH | H | H | CF₃ | C | N | CH | CH | 5.2 ± 0.9 | >113 | >22 |
| 30 | fS | 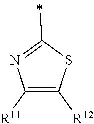 | H | H | H | H | CHOHCH₂OH | H | H | CF₃ | C | N | CH | CH | 3.8 ± 0.2 | >113 | >30 |
| 31 | racemate | 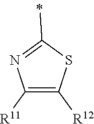 | CH₃ | H | H | H | CH₂OH | H | CH₃ | CF₃ | C | N | CH | CH | 2.6 ± 0.4 | >68.3 | >26 |
| 32 | racemate | 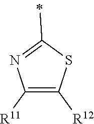 | H | H | H | H | CH₂OH | H | CH₃ | CF₃ | C | N | CH | CH | 5.1 ± 0.4 | >70.5 | >14 |
| 33 | fR | 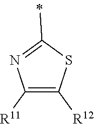 | CH₃ | H | H | H | CH₂OH | H | H | CF₃ | C | N | CH | CH | 0.35 | 85 ± 3 | 243 |
| 34 | fS | 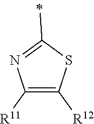 | CH₃ | H | H | H | CH₂OH | H | H | CF₃ | C | N | CH | CH | 1.1 | 85 ± 4 | 77 |
| 35 | fR | 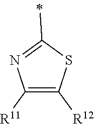 | H | H | H | H | CH₂OH | CH₂OH | H | CF₃ | C | N | CH | CH | 1.8 | >113 | 63 |
| 36 | fS | 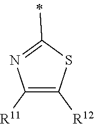 | H | H | H | H | CH₂OH | CH₂OH | H | CF₃ | C | N | CH | CH | 13 | >113 | 9 |
| 37 | fR | 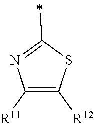 | H | H | H | H | H | H | H | CF₃ | C | N | CH | CH | 2 ± 0.2 | 89.3 ± 1 | 45 |
| 38 | fS | 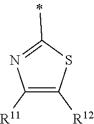 | H | H | H | H | H | H | H | CF₃ | C | N | CH | CH | 1.2 ± 0.03 | 93.6 ± 1.5 | 78.0 |
| 39 | fR | 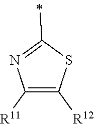 | H | H | H | H | CH₂OH | H | H | Cl | C | CH | N | CH | >11 | >132 | −12 |
| 40 | fS | 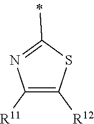 | H | H | H | H | CH₂OH | H | H | Cl | C | CH | N | CH | >11 | >132 | −12 |
| 41 | fR | 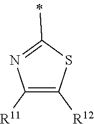 | H | H | H | H | CH₂OH | H | H | CF₃ | C | CH | N | CH | 12.7 ± 2.8 | >122 | >9.6 |
| 42 | fS | 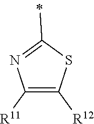 | H | H | H | H | CH₂OH | H | H | CF₃ | C | CH | N | CH | 11.9 ± 1.9 | >122 | >10.2 |
| 43 | fR | 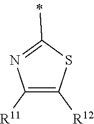 | H | H | H | H | CH₂OH | H | H | CF₃ | C | N | N | CH | >12 | >73 | ~6 |
| 44 | fS | 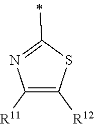 | H | H | H | H | CH₂OH | H | H | CF₃ | C | N | N | CH | >12 | >73 | ~6 |
| 45 | fR | 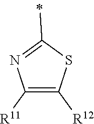 | H | H | H | H | CH₂OH | H | H | Cl | C | N | CH | N | >15.8 | >66 | −4 |
| 46 | fS | 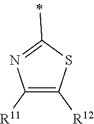 | H | H | H | H | CH₂OH | H | H | Cl | C | N | CH | N | >15.8 | >66 | −4 |
| 47 | fR | 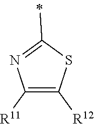 | H | H | H | OCH₃ | CH₂OH | H | H | | N | CH | CH | CH | >11 | >133 | −12 |
| 48 | fS | 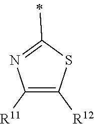 | H | H | H | OCH3 | CH₂OH | H | H | | N | CH | CH | CH | >11 | >133 | −12 |
| 49 | fR | 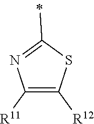 | H | H | H | H | CH₂OH | H | H | Cl | C | CH | CH | N | 2.7 ± 0.7 | >132 | >48.9 |
| 50 | fS | 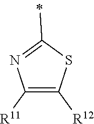 | H | H | H | H | CH₂OH | H | H | Cl | C | CH | CH | N | 0.85 ± 0.1 | >132 | >155 |
| 51 | fR | 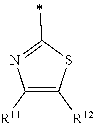 | H | H | H | H | CH₂OH | H | H | CF₃ | C | CH | CH | N | >25.4 | >102 | >4 |
| 52 | fS | 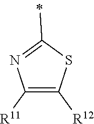 | H | H | H | H | CH₂OH | H | H | CF₃ | C | CH | CH | N | >25.4 | >102 | >4 |
| 53 | fR | 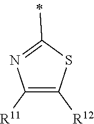 | H | CH₃CH₂ | H | H | CH₂OH | H | H | CF₃ | C | N | N | CH | 5 ± 0.2 | 50.8 ± 4.7 | 10.2 |
| 54 | fS | 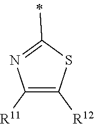 | H | CH₃CH₂ | H | H | CH₂OH | H | H | CF₃ | C | N | N | CH | 4.5 ± 0.5 | 37.9 ± 6.9 | 18.4 |
| 55 | fR | 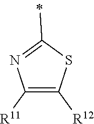 | CH₃ | H | H | H | CH₂OH | H | H | CF₃ | C | CH | CH | N | 10 ± 1.2 | −122.7 | −12.3 |
| 56 | fS | 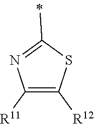 | CH₃ | H | H | H | CH₂OH | H | H | CF₃ | C | CH | CH | N | 7.6 ± 2.1 | −122.7 | −16.1 |
| | | | | | | | | | | | | | | | μM | | |
| No. | Enantiomer | R^{Y} | R² | R³ | R⁴ | R¹⁰ | R¹⁴ | R¹³ | R¹⁷ | R¹⁸ | W | X | Y | Z | IC₅₀ | CC₅₀ | SI |
| 57 | fR | 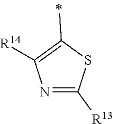 | H | H | H | H | H | CH₂OH | H | CF₃ | C | CH | CH | N | 2.1 ± 0.4 | >122 | >58 |
| 58 | fS | 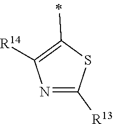 | H | H | H | H | H | CH₂OH | H | CF₃ | C | CH | CH | N | 2.1 ± 0.2 | >122 | >58 |
| 59 | fR | 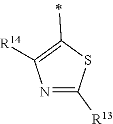 | H | H | H | H | H | CH₂OH | H | Cl | C | CH | CH | N | ~26 | >106 | — |
| 60 | fS | 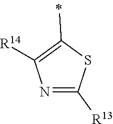 | H | H | H | H | H | CH₂OH | H | Cl | C | CH | CH | N | 3.6 ± 0.4 | >106 | >29.4 |
| 61 | fR | 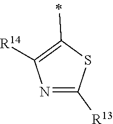 | CH₃ | H | H | H | H | CH₂OH | H | CF₃ | C | CH | CH | N | 2.3 ± 1 | 65 ± 4.9 | 28.3 |
| 62 | fS | 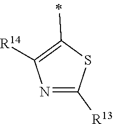 | CH₃ | H | H | H | H | CH₂OH | H | CF₃ | C | CH | CH | N | 1.9 ± 0.25 | 63.8 ± 2.1 | 9.8 |
| | | | | | | | | | | | | | | | μM | | |
| No. | Enantiomer | R^{Y} | R² | R³ | R⁴ | R¹⁰ | R²⁰ | R¹⁹ | R¹⁷ | R¹⁸ | W | X | Y | Z | IC₅₀ | CC₅₀ | SI |
| 63 | fR | 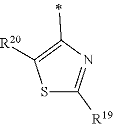 | CH₃ | H | H | H | H | CH₂OH | H | CF₃ | C | CH | CH | N | 1.4 ± 0.1 | 42.3 ± 0.2 | 30.2 |
| 64 | fS | 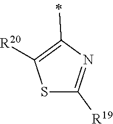 | CH₃ | H | H | H | H | CH₂OH | H | CF₃ | C | CH | CH | N | 1.3 ± 0.2 | 47.8 ± 5.9 | 36.8 |
| 65 | fR | 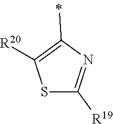 | H | H | H | H | H | CH₂OH | H | CF₃ | C | CH | CH | N | 1.6 ± 0.2 | 84.3 ± 1.4 | 52.7 |
| 66 | fS | 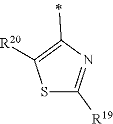 | H | H | H | H | H | CH₂OH | H | CF₃ | C | CH | CH | N | 1.7 ± 0.2 | 85.1 ± 2.5 | 50 |
| 67 | fR | 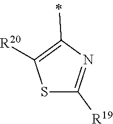 | H | H | H | H | H | CH₂OH | H | Cl | C | CH | CH | N | 7 . . .6 ± 1.6 | >106 | >13.9 |
| 68 | fS | 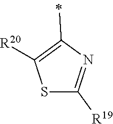 | H | H | H | H | H | CH₂OH | H | Cl | C | CH | CH | N | 8.2 ± 2.1 | >106 | >12.9 |
| 69 | fR | 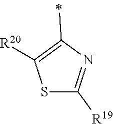 | H | H | H | H | H | C(=O)NHCH₂CH₂OH | H | CF₃ | C | CH | CH | N | >21.3 | >106.7 | — |
| 70 | fS | 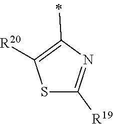 | H | H | H | H | H | C(=O)NHCH₂CH₂OH | H | CF₃ | C | CH | CH | N | >21.3 | >106.7 | — |

### Example 2. Antiviral Activity of the Polycyclic Subject Compounds Against a Large Panel of HIV-1 Env-Pseudotyped Reference Viruses

Compounds 8 and 33 were evaluated against a panel of HIV-1 Env pseudotypes based on a selection of 49 HIV-1 clones of clinical isolates of subtype A, B, C, D and of subtype A recombinant (Arec) HIV-1 clones including A/D, A2/D, and AG. These compounds exhibited sub-micromolar anti-HIV-1 activity (Table 2) with the overall mean of the IC50s very similar to those observed in the pseudovirus HIV-1 HXB2 assay above.

TABLE 2
Neutralization activity of subject compounds against a panel of HIV-1 Env Pseudoviruses

| | | | IC₅₀ μM^{a} | | |
|---|---|---|---|---|---|
| Subtype | NIH # | ENVs | Comparator B | Compound 33 | Compound 8 |
| A | 11887 | Q259ENV.W6 | 0.46 ± 0.06^{c} | 0.4 ± 0.05 | 0.23 ± 0.03 |
| | 11888 | QB726.70M.ENV.C4 | 0.31 ± 0.07 | 0.63 ± 0.1 | 0.22 ± 0.01 |
| | 11890 | QF495.23M.ENV.A1 | 0.44 ± 0.05 | 0.47 ± 0.05 | 0.14 ± 0.003 |
| | 11891 | QF495.23M.ENV.A3 | 0.26 ± 0.04^{c} | 0.71 ± 0.17 | 0.33 ± 0.01 |
| | | BG505-T332N | 0.41 ± 0.03 | 0.27 ± 0.007 | 0.124 ± 0.005 |
| | | KNH1144 | 0.62 ± 0.08 | 0.74 ± 0.06 | 0.26 ± 0.02 |
| A/D | 11901 | QA790.204I.ENV.A4 | 0.29 ± 0.01 | 0.36 ± 0.03 | 0.14 ± 0.003 |
| | 11904 | QA790.204I.ENV.E2 | 0.41 ± 0.03 | 0.36 ± 0.02 | 0.16 ± 0.03 |
| A2/D | 11905 | QG393.60M.ENV.A1 | 0.34 ± 0.02 | 0.42 ± 0.03 | 0.22 ± 0.002 |
| | 11906 | QG393.60M.ENV.B7 | 0.6 ± 0.005 | 0.5 ± 0.04 | 0.17 ± 0.03 |
| A/G | 11591 | CRF02_AG Clone 211 | 0.58 ± 0.05 | 0.38 ± 0.04 | 0.23 ± 0.04 |
| | 11594 | CRF02_AG clone 250 | 0.41 ± 0.01 | 0.42 ± 0.01 | 0.21 ± 0.01 |
| | 11595 | CRF02_AG clone 251 | 0.36 ± 0.06 | 0.39 ± 0.03 | 0.12 ± 0.01 |
| | 11598 | CRF02_AG clone 255 | 0.33 ± 0.01 | 0.66 ± 0.03 | 0.22 ± 0.01 |
| | 11599 | CRF02_AG clone 257 | 0.39 ± 0.01 | 0.74 ± 0.1 | 0.16 ± 0.002 |
| | 11600 | CRF13_cpx clone 258 | 0.52 ± 0.07 | 0.52 ± 0.1 | 0.14 ± 0.003 |
| | 11602 | CRF02_AG clone 266 | 0.51 ± 0.06 | 0.75 ± 0.05 | 0.23 ± 0.01 |
| AE | 11603 | CRF01_AE clone 269 | 0.52 ± 0.02 | 0.41 ± 0.2 | 0.138 ± 0.01 |
| B | | B41 | 0.36 ± 0.04 | 0.38 ± 0.05 | 0.136 ± 0.001 |
| | 11018 | QH0692, clone 42 | 0.27 ± 0.01^{c} | 0.99 ± 0.05 | 0.28 ± 0.02 |
| | 11022 | PVO, clone 4 | 0.41 ± 0.06 | 0.33 ± 0.04 | 0.11 ± 0.03 |
| | 11023 | TRO, clone 11 | 0.39 ± 0.02 | 0.45 ± 0.03 | 0.23 ± 0.005 |
| | 11036 | RHPA4259 clone 7 | 0.37 ± 0.07 | 0.24 ± 0.09 | 0.15 ± 0.01 |
| | 11037 | THRO4156 clone 18 | 0.32 ± 0.02 | 0.15 ± 0.03 | 0.16 ± 0.02 |
| | 11038 | CAAN5342 clone A2 | 0.22 ± 0.03 | 0.38 ± 0.07 | 0.13 ± 0.005 |
| | 11058 | SC422661.8 | 0.32 ± 0.08 | 0.16 ± 0.01 | 0.13 ± 0.01 |
| | 11560 | 1006_11.C3.1601 | 0.58 ± 0.06 | 0.27 ± 0.01 | 0.139 ± 0.003 |
| | 11561 | 1054.TC4.1499 | 0.25 ± 0.02 | 0.39 ± 0.02 | 0.23 ± 0.01 |
| | 11562 | 1056.TA11.1826 | 0.58 ± 0.01 | 0.71 ± 0.1 | 0.13 ± 0.006 |
| | 11563 | 1058 11.B11.1550^{b} | 0.29 ± 0.02 | 0.49 ± 0.06 | 0.28 ± 0.01 |
| | 11572 | 9021_14.B2.4571 | 0.36 ± 0.08 | 0.57 ± 0.13 | 0.11 ± 0.03 |
| | 11578 | WEAUd15.410.5017^{b} | 0.76 ± 0.04 | 0.54 ± 0.04 | 0.22 ± 0.002 |
| C | 11307 | Du172, clone 17 | 0.3 ± 0.03 | 0.27 ± 0.02 | 0.19 ± 0.002 |
| | 11308 | Du422, clone 1 | 0.63 ± 0.09 | 0.42 ± 0.01 | 0.23 ± 0.01 |
| | 11309 | ZM197M.PB7, SVPC6 | 0.27 ± 0.01 | 0.31 ± 0.03 | 0.13 ± 0.006 |
| | 11310 | ZM214M.PL15, SVPC7 | 0.24 ± 0.007 | 0.26 ± 0.005 | 0.13 ± 0.005 |
| | 11312 | ZM249M.PL1, SVPC10 | 0.55 ± 0.07 | 0.44 ± 0.1 | 0.18 ± 0.01 |
| | 11313 | ZM53M.PB12, SVPC11 | 0.54 ± 0.03 | 0.57 ± 0.3 | 0.28 ± 0.002 |
| | 11314 | ZM109F.PB4 | 0.29 ± 0.01 | 0.47 ± 0.01 | 0.28 ± 0.003 |
| | 11317 | CAP210.2.00.E8, SVPC17 | 0.47 ± 0.03 | 0.14 ± 0.05 | 0.115 ± 0.002 |
| | 11502 | HIV-16055-2, clone 3 | 0.29 ± 0.06 | 0.29 ± 0.09 | 0.12 ± 0.006 |
| | 11504 | HIV-16936-2, clone 21 | 0.47 ± 0.05 | 0.48 ± 0.1 | 0.21 ± 0.04 |
| | 11506 | HIV-25711-2, clone 4 | 0.26 ± 0.02 | 0.32 ± 0.03 | 0.17 ± 0.01 |
| | 11507 | HIV-225925-2, clone 22 | 0.24 ± 0.02 | 0.46 ± 0.04 | 0.17 ± 0.01 |
| | 11908 | QB099.391M.ENV.B1 | 0.42 ± 0.05 | 0.33 ± 0.05 | 0.118 ± 0.002 |
| D | 11911 | QA013.70I.ENV.H1 | 0.32 ± 0.01 | 0.55 ± 0.04 | 0.25 ± 0.01 |
| | 11912 | QA013.70I.ENV.M12 | 0.22 ± 0.01 | 0.25 ± 0.02 | 0.16 ± 0.02 |
| | 11916 | QD435.100M.ENV.B5 | 0.3 ± 0.01 | 0.37 ± 0.03 | 0.13 ± 0.01 |
| | 11918 | QD435.100M.ENV.E1 | 0.19 ± 0.04 | 0.24 ± 0.01 | 0.136 ± 0.002 |
| G | 11596 | CRF02_G clone 252 | 0.26 ± 0.03 | 0.13 ± 0.01 | 0.11 ± 0.01 |

| | | | |
|---|---|---|---|
| Mean ± SEM (μM): Overall (n = 50) | 0.39 ± 0.02 | 0.43 ± 0.02 | 0.18 ± 0.008 |
| SI | 108.7 | 198.4 | 607.2 |
| Subtype A (n = 6) | 0.42 ± 0.05 | 0.54 ± 0.08 | 0.22 ± 0.003 |
| SI | 101 | 158 | 496.8 |
| Subtype A_{rec} (n = 12) | 0.44 ± 0.03 | 0.49 ± 0.04 | 0.18 ± 0.01 |
| SI | 96.4 | 174.1 | 607.2 |
| Subtype B (n = 14) | 0.39 ± 0.04 | 0.43 ± 0.06 | 0.17 ± 0.02 |
| SI | 108.7 | 198.4 | 642.9 |
| Subtype C (n = 13) | 0.38 ± 0.04 | 0.37 ± 0.03 | 0.18 ± 0.02 |
| SI | 111.6 | 230.5 | 607.2 |
| Subtype D (n = 4) | 0.26 ± 0.03 | 0.35 ± 0.07 | 0.17 ± 0.03 |
| SI | 163.1 | 243.7 | 642.9 |

^{a}The reported IC₅₀ values represent the means ± standard deviations (n = 3).
^{b}R5X4-tropic virus all the rest are CCR5-tropic viruses.
^{c}Data previously published: Curreli et al.,. Eur J Med Chem 154: 367-391, 2018.
^{d}SI = CC₅₀/IC₅₀; CC₅₀ values from Table 1.

### Example 3. The Polycyclic Compounds do not Enhance HIV-1 Entry into CD4-Negative Cells

Compound C is known to be an entry agonist promoting CCR5 binding and enhancing HIV-1 entry into CD4-negative cells expressing CCR5 (Curreli, F. et al., Antimicrob Agents Chemother 58:5478-5491, 2014; Schon, A. et al., Biochemistry 45:10973-10980, 2006). To test if the subject compounds exhibit this undesirable trait and behave as entry antagonist, CD4-negative and CCR5-positive cells, Cf2TH-CCR5, were infected with recombinant CD4-dependent HIV-1_{ADA} virus in the presence of escalating concentrations of compounds 8 and 33. Compound C was used as a control.

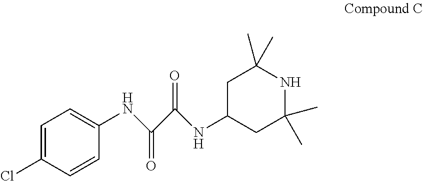 [SMILES: CC1(C)CC(NC(=O)C(=O)Nc2ccc(Cl)cc2)CC(C)(C)N1]

CD4-negative Cf2Th-CCR5 cells were plated at 6×10³ cells/well in a 96-well tissue culture plate and incubated overnight. The cells were infected with the luciferase-expressing recombinant CD4-dependent pseudovirus HIV-1_{ADA} as previously described (Si, Z. et al., Proc Natl Acad Sci USA 101:5036-5041, 2004). Briefly, following overnight incubation, aliquots of HIV-1_{ADA} pseudovirus pre-treated with graded concentrations of the subject or control compounds for 30 min, were added to the cells and cultured for 48 h. Cells were washed with PBS and lysed with 40 μl of cell lysis reagent. Lysates were transferred to a white 96-well plate and mixed with 100 μl of luciferase assay reagent. The luciferase activity was immediately measured to obtain the relative infection compared to the untreated control. The Relative virus infectivity indicates the amount of infection detected in the presence of the compounds divided by the amount of infection detected in the absence of the compounds.

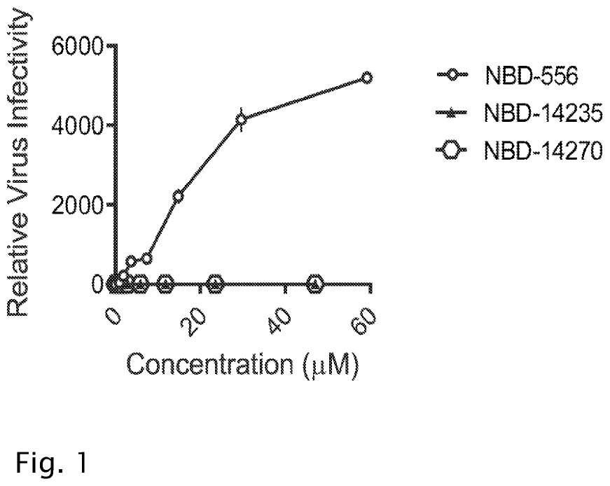

Although Compound C enhanced the infection of the Cf2Th-CCR5 cells, neither Compound 8 or Compound 33 enhanced HIV-1 infectivity in these cells, indicating that the HIV-1 entry antagonist property was maintained by these compounds (FIG. 1).

### Example 4. Assay Procedures

### Pseudovirus Preparation

Pseudoviruses capable of single cycle infection were prepared as previously described (Curreli, F. et al., Antimicrob Agents Chemother 58:5478-5491, 2014). Briefly, 5×106 HEK293T cells were transfected with a solution containing the same amounts of an HIV-1 Env-deleted pro-viral backbone plasmid pSG3Δenv DNA or pNL4-3.Luc.R-.E-DNA and an HIV-1 Env-expression plasmid with FuGENE HD (Promega). VSV-G pseudovirus was prepared by transfecting the HEK 293T cells with a combination of the Env-expressing plasmid pVPack-VSV-G, the MLV gag-pol-expressing plasmid pVPack-GP, the pFB-luc plasmid and FuGENE HD. Pseudovirus-containing supernatants were collected two days after transfection, filtered, tittered and stored.

### Measurement of Antiviral Activity

Single-cycle infection assay in TZM-bl cells. The subject compounds were evaluated in single-cycle infection assay for their anti-HIV-1 activity by infecting TZM-bl cells with an HIV-1 pseudovirus expressing the Env from the lab-adapted HIV-1 HXB-2 (X4). Also, the subject compounds were tested against a large number of HIV-1 pseudotyped viruses expressing the Env from a panel of diverse clinical isolates as previously described (Curreli, F. et al., Antimicrob Agents Chemother 58:5478-5491, 2014). To this end, TZM-bl cells were platted at 1×10⁴/well in a 96-well tissue culture plate and cultured. Following overnight incubation, aliquots of HIV-1 pseudoviruses were pre-treated with graded concentrations of the subject compounds for 30 min and added to the cells. Following 3 days of incubation, the cells were washed and lysed. 20 μl of the lysates were transferred to a white plate and mixed with the luciferase assay reagent (Promega). The luciferase activity was immediately measured with a Tecan infinite M1000 reader, and the percent inhibition by the compounds and IC₅₀ (the half maximal inhibitory concentration) values were calculated using the GraphPad Prism software.

### Evaluation of Cytotoxicity

TZM-bl cells. The cytotoxicity of the small polycyclic molecules in TZM-bl cells was determined by using the colorimetric method CellTiter 96® AQueous One Solution Cell Proliferation Assay (MTS) (Promega) following the manufacturer's instructions. Briefly, TZM-bl cells were platted in a 96-well tissue culture plate at 1×10⁴/well and cultured at 37° C. Following overnight incubation, the cells were incubated with 100 μl of the compounds at graded concentrations and cultured for 3 days. The MTS reagent was added to the cells and incubated for 4 h at 37° C. The absorbance was recorded at 490 nm. The percent of cytotoxicity and the CC₅₀ (the concentration for 50% cytotoxicity) values were calculated as above.

### Assay in Cf2Th-CCR5 Cells

CD4-negative Cf2Th-CCR5 cells were infected with the luciferase-expressing recombinant CD4-dependent pseudovirus HIV-1ADA as previously described (F. Curreli, Y. D. Kwon, D. S. Belov, R. R. Ramesh, A. V. Kurkin, A. Altieri, P. D. Kwong, A. K. Debnath, J Med Chem 2017, 60, 3124-3153). Briefly, the Cf2Th-CCR5 cells were plated at 6×10³ cells/well in a 96-well tissue culture plate. Following overnight incubation, aliquots of the pseudovirus HIV-1ADA were pre-treated with graded concentrations of the small polycyclic molecules for 30 min then, added to the cells and cultured for 48 hours. Cells were washed with PBS and lysed with 40 μl of cell lysis reagent. Lysates were transferred to a white 96-well plate and mixed with 100 μl of luciferase assay reagent. The luciferase activity was immediately measured to obtain the relative infection concerning the untreated control. The Relative virus infectivity indicates the amount of infection detected in the presence of the compounds divided by the amount of infection detected in the absence of the compounds.

### Example 5. Synthetic Methods

### Example 5.1

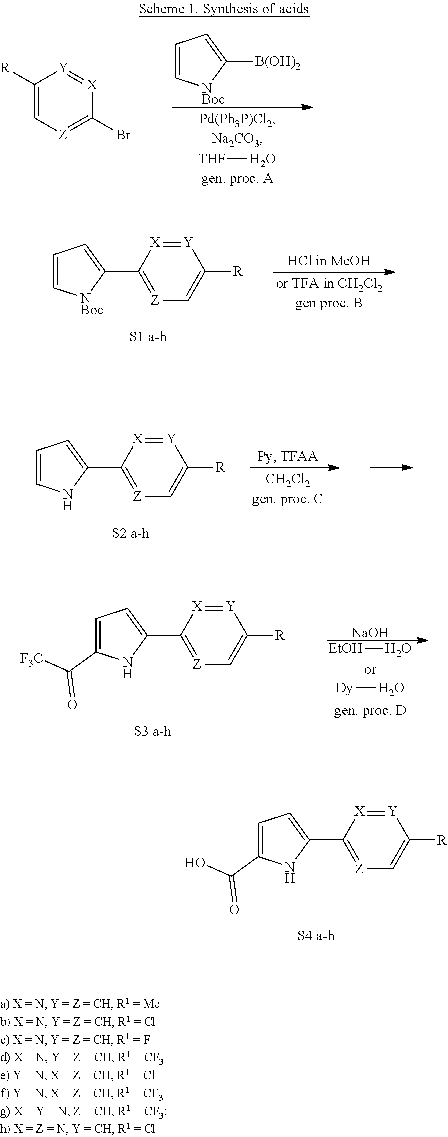

### Example 5.2 General Procedure A: For Suzuki Coupling

To a solution containing appropriate bromide (50 mmol, 1 equiv), (1-(tert-butoxycarbonyl)-1H-pyrrol-2-yl)boronic acid (50 mmol, 1 equiv) in THF-H₂O (1:1, 100 mL), Na₂CO₃ (100 mmol, 2 equiv) and Pd(Ph₃P)Cl₂ (1 mol. %) were added under a nitrogen atmosphere. The mixture was stirred at reflux for 8-15 h (TLC-control). After cooling to the room temperature, water (50 mL) and CH₂Cl₂ (50 mL) were added. The organic layer was separated, the aqueous layer was extracted with CH₂Cl₂ (3×50 mL). The combined organic layers were dried over Na₂SO₄, filtered, and concentrated. Purification by flash chromatography using hexane-EtOAc mixture as eluent afforded desired compound. Compound S1a-h were obtained following the general procedure A.

### Example 5.3 Tert-butyl 2-(5-methylpyridin-2-yl)-1H-pyrrole-1-carboxylate (S1a)

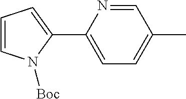 [SMILES: Cc1ccc(-c2cccn2C(=O)OC(C)(C)C)nc1]

Eluent: Hex-EtOAc (from 10:1 to 5:1), Rf=0.2 (5:1, Hex-EtOAc). Yield=63%.

¹H NMR (CDCl₃, 400 MHz): δ=1.37 (s, 9H), 2.34 (s, 3H), 6.22 (t, J=3.3 Hz, 1H), 6.36 (dd, J=3.2, 1.7 Hz, 1H), 7.28 (d, J=8.1 Hz, 1H), 7.33 (dd, J=3.2, 1.8 Hz, 1H), 7.48 (dd, J=7.9, 1.8 Hz, 1H), 8.42-8.45 (m, 1H).

¹³C NMR (CDCl₃, 100 MHz): δ=18.3, 27.6 (3C), 83.5, 110.5, 115.3, 123.2, 123.2, 131.3, 134.2, 136.3, 149.2, 149.3, 150.2.

### Example 5.4 Tert-butyl 2-(5-chloropyridin-2-yl)-1H-pyrrole-1-carboxylate (S1b)

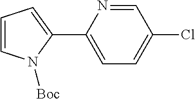 [SMILES: CC(C)(C)OC(=O)n1cccc1-c1ccc(Cl)cn1]

Eluent: Hex-EtOAc (from 20:1 to 10:1), Rf=0.3 (10:1, Hex-EtOAc). Yield=84%.

¹H NMR (CDCl₃, 400 MHz): δ=1.41 (s, 9H), 6.25 (t, J=3.3 Hz, 1H), 6.43 (dd, J=3.3, 1.7 Hz, 1H), 7.34-7.38 (m, 2H), 7.66 (dd, J=8.4, 2.5 Hz, 1H), 8.57 (d, J=2.4 Hz, 1H).

¹³C NMR (CDCl₃, 100 MHz): δ=27.8 (3C), 84.1, 110.8, 116.3, 124.0, 124.3, 130.2, 133.1, 135.6, 147.8, 149.2, 151.0.

### Example 5.5 Tert-butyl 2-(5-fluoropyridin-2-yl)-1H-pyrrole-1-carboxylate (Sic)

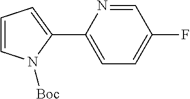 [SMILES: CC(C)(C)OC(=O)n1cccc1-c1ccc(F)cn1]

Eluent: Hex-EtOAc (from 20:1 to 10:1), Rf=0.3 (10:1, Hex-EtOAc). Yield=68%.

¹H NMR (CDCl₃, 400 MHz): δ=1.39 (s, 9H), 6.23 (t, J=3.3 Hz, 1H), 6.39 (dd, J=3.3, 1.7 Hz, 1H), 7.35 (dd, J=3.2, 1.7 Hz, 1H), 7.39 (d, J=1.8 Hz, 1H), 7.40-7.42 (m, 1H), 8.47 (t, J=1.7 Hz, 1H).

¹³C NMR (CDCl₃, 100 MHz): δ=27.7 (3C), 83.9, 110.6, 115.8, 122.7 (d, J=18.6 Hz), 123.6, 124.6 (d, J=4.2 Hz), 133.1, 137.0 (d, J=23.8 Hz), 149.2 (d, J=1.5 Hz), 149.3, 158.4 (d, J=256.0 Hz).

### Example 5.6 Tert-butyl 2-(5-(trifluoromethyl)pyridin-2-yl)-1H-pyrrole-1-carboxylate (S1d)

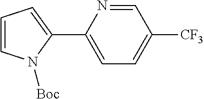 [SMILES: CC(C)(C)OC(=O)n1cccc1-c1ccc(C(F)(F)F)cn1]

Eluent: Hex-EtOAc (from 20:1 to 10:1), Rf=0.3 (Hex-EtOAc). Yield=65%.

¹H NMR (CDCl₃, 400 MHz): 1.42 (s, 9H), 6.28 (t, J=3.3 Hz, 1H), 6.54 (dd, J=3.4, 1.7 Hz, 1H), 7.40 (dd, J=3.2, 1.7 Hz, 1H), 7.54 (d, J=8.2 Hz, 1H), 7.92 (dd, J=8.3, 2.2 Hz, 1H), 8.87 (s, 1H).

¹³C NMR (CDCl₃, 100 MHz): δ=27.6 (3C), 84.3, 110.9, 117.3, 122.8, 123.8 (q, J=272.0 Hz), 124.4 (q, J=33.0 Hz), 124.8, 132.9 (q, J=3.5 Hz), 133.0, 145.8 (q, J=4.1 Hz), 149.1, 156.0 (q, J=1.5 Hz).

### Example 5.7 Tert-butyl 2-(6-chloropyridin-3-yl)-1H-pyrrole-1-carboxylate (S1e)

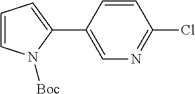 [SMILES: CC(C)(C)OC(=O)n1cccc1-c1ccc(Cl)nc1]

Eluent: Hex-EtOAc (from 20:1 to 10:1), Rf=0.4 (10:1, Hex-EtOAc). Yield=76%.

¹H NMR (CDCl₃, 400 MHz): δ=1.44 (s, 9H), 6.25-6.29 (m, 2H), 7.33 (d, J=8.2 Hz, 1H), 7.41 (t, J=2.5 Hz, 1H), 7.66 (dd, J=8.2, 2.5 Hz, 1H), 8.38 (d, J=2.3 Hz, 1H).

¹³C NMR (CDCl₃, 100 MHz): δ=27.8 (3C), 84.6, 111.1, 116.1, 123.1, 123.8, 129.3, 130.0, 139.4, 149.0, 149.5, 150.0.

### Example 5.8 Tert-butyl 2-(6-(trifluoromethyl)pyridin-3-yl)-1H-pyrrole-1-carboxylate (S1f)

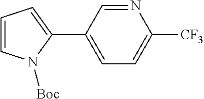 [SMILES: CC(C)(C)OC(=O)n1cccc1-c1ccc(C(F)(F)F)nc1]

Eluent: Hex-EtOAc (from 30:1 to 20:1), Rf=0.5 (30:1, Hex-EtOAc). Yield=84%.

¹H NMR (CDCl₃, 400 MHz): δ=1.43 (s, 9H), 6.30 (t, J=3.3 Hz, 1H), 6.32-6.35 (m, 1H), 7.45 (dd, J=3.2, 1.8 Hz, 1H), 7.68 (d, J=8.1 Hz, 1H), 7.86 (dd, J=8.1, 1.8 Hz, 1H), 8.72 (d, J=1.7 Hz, 1H).

¹³C NMR (CDCl₃, 400 MHz): δ=27.7 (3C), 84.8, 111.3, 116.8, 119.5 (q, J=2.8 Hz), 121.8 (q, J=273.9 Hz), 124.3, 130.0, 133.2, 137.3, 146.3 (q, J=34.8 Hz), 148.9, 150.0.

### Example 5.9 Tert-butyl 2-(6-(trifluoromethyl)pyridazin-3-yl)-1H-pyrrole-1-carboxylate (S1g)

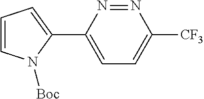 [SMILES: CC(C)(C)OC(=O)n1cccc1-c1ccc(C(F)(F)F)nn1]

Eluent: Hex-EtOAc (from 20:1 to 5:1), Rf=0.4 (5:1, Hex-EtOAc). Yield=61%.

¹H NMR (CDCl₃, 400 MHz): δ=1.45 (s, 9H), 6.35 (t, J=3.4 Hz, 1H), 6.73 (dd, J=3.4, 1.6 Hz, 1H), 7.48 (dd, J=3.2, 1.7 Hz, 1H), 7.75-7.79 (m, 2H).

¹³C NMR (CDCl₃, 400 MHz): δ=27.8 (3C), 85.1, 111.6, 119.2, 121.7 (q, J=274.2 Hz), 122.8 (q, J=2.4 Hz), 125.8, 127.7, 129.9, 148.9, 149.5 (q, J=35.0 Hz), 157.2.

### Example 5.10 Tert-butyl 2-(5-chloropyrimidin-2-yl)-1H-pyrrole-1-carboxylate (S1h)

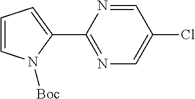 [SMILES: CC(C)(C)OC(=O)n1cccc1-c1ncc(Cl)cn1]

Eluent: Hex-EtOAc (from 30:1 to 20:1), Rf=0.5 (30:1, Hex-EtOAc). Yield=58%.

¹H NMR (CDCl₃, 400 MHz): δ=1.44 (s, 9H), 6.26 (t, J=3.3 Hz, 1H), 6.78 (dd, J=3.4, 1.7 Hz, 1H), 7.35 (dd, J=3.1, 1.7 Hz, 1H), 8.67 (s, 2H).

¹³C NMR (CDCl₃, 400 MHz): δ=27.8 (3C), 84.3, 110.9, 118.8, 125.7, 128.4, 131.8, 149.2, 155.2 (2C), 158.8.

### Example 5.11 General Procedure B: For Boc-Deprotection

To a solution containing Boc-protected compound (30 mmol) in MeOH (15 mL, 2M solution), 1M HCl solution in MeOH (45 mL) was added in a one portion. The mixture was stirred at reflux for 7-8 h. After cooling to the room temperature, solvent was evaporated. Then 10% aqueous K₂CO₃ (50 mL) was added carefully (CO₂ evolution) and mixture was extracted with CH₂Cl₂ (3×50 mL). The combined organic layers were dried over Na₂SO₄, filtered, and concentrated. Crude product was used in the next step without purification. Compound S2a-h were synthesized following the general procedure B.

### Example 5.12 5-Methyl-2-(1H-pyrrol-2-yl)pyridine (S2a)

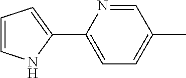

Yield=94%.

¹H NMR (CDCl₃, 400 MHz): δ=2.33 (s, 3H), 6.31-6.35 (m, 1H), 6.72-6.75 (m, 1H), 6.89-6.91 (m, 1H), 7.48 (dd, J=8.2, 1.9 Hz, 1H), 7.53 (d, J=8.1 Hz, 1H), 8.27-8.38 (m, 1H), 10.73 (br. s., 1H).

¹³C NMR (CDCl₃, 100 MHz): δ=18.2, 106.8, 109.9, 118.1, 119.9, 129.9, 131.8, 137.4, 148.5, 148.9.

### Example 5.13 5-Chloro-2-(1H-pyrrol-2-yl)pyridine (S2b)

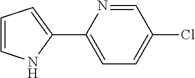 [SMILES: Clc1ccc(-c2ccc[nH]2)nc1]

Yield=92%.

¹H NMR (CDCl₃, 400 MHz): δ=6.31-6.34 (m, 1H), 6.71-6.74 (m, 1H), 6.92 (td, J=2.6, 1.4 Hz, 1H), 7.50 (d, J=8.6 Hz, 1H), 7.60 (dd, J=8.6, 2.4 Hz, 1H), 8.42 (d, J=2.2 Hz, 1H), 9.90 (br. s., 1H).

¹³C NMR (CDCl₃, 100 MHz): δ=107.9, 110.6, 118.9, 120.5, 128.3, 130.7, 136.4, 147.7, 148.9.

### Example 5.14 5-Fluoro-2-(1H-pyrrol-2-yl)pyridine (S2c)

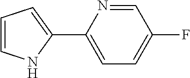 [SMILES: Fc1ccc(-c2ccc[nH]2)nc1]

Yield=93%.

¹H NMR (CDCl₃, 400 MHz): δ=6.33 (dd, J=6.2, 2.7 Hz, 1H), 6.66-6.71 (m, 1H), 6.88-6.94 (m, 1H), 7.38 (td, J=8.5, 2.8 Hz, 1H), 7.56 (dd, J=8.8, 4.3 Hz, 1H), 8.35 (d, J=2.8 Hz, 1H), 9.96 (br. s., 1H).

¹³C NMR (CDCl₃, 100 MHz): δ=107.1, 110.3, 119.1 (d, J=4.1 Hz), 120.2, 123.9 (d, J=19.3 Hz), 130.9, 136.7 (d, J=23.9 Hz), 147.4 (d, J=3.2 Hz), 157.8 (d, J=253.2 Hz).

### Example 5.15 2-(1H-Pyrrol-2-yl)-5-(trifluoromethyl)pyridine (S2d)

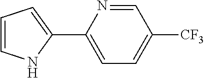 [SMILES: FC(F)(F)c1ccc(-c2ccc[nH]2)nc1]

Eluent: Hex-EtOAc (from 20:1 to 10:1), Rf=0.5 (10:1, Hex-EtOAc). Yield=88%.

¹H NMR (CDCl₃, 400 MHz): δ=6.32-6.37 (m, 1H), 6.80-6.86 (m, 1H), 6.94-7.03 (m, 1H), 7.61 (d, J=8.4 Hz, 1H), 7.83 (dd, J=8.4, 2.1 Hz, 1H), 8.71 (s, 1H), 9.74 (br. s., 1H).

¹³C NMR (CDCl₃, 100 MHz): δ=109.6, 111.0, 117.6, 121.6 122.9 (q, J=33.0 Hz), 124.0 (q, J=271.6 Hz), 130.5, 133.7 (q, J=3.5 Hz), 146.1 (q, J=4.4 Hz), 153.5 (q, J=1.5 Hz).

### Example 5.16 2-Chloro-5-(1H-pyrrol-2-yl)pyridine (S2e)

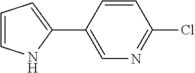 [SMILES: Clc1ccc(-c2ccc[nH]2)cn1]

Yield=93%.

¹H NMR (CDCl₃, 400 MHz): δ=6.33-6.37 (m, 1H), 6.57-6.60 (m, 1H), 6.94-6.98 (m, 1H), 7.32 (d, J=8.4 Hz, 1H), 7.74 (dd, J=8.3, 2.6 Hz, 1H), 8.52 (d, J=2.4 Hz, 1H), 8.84 (br. s., 1H).

¹³C NMR (CDCl₃, 100 MHz): δ=107.9, 110.6, 120.8, 124.5, 127.4, 128.2, 134.2, 144.6.

### Example 5.17 5-(1H-Pyrrol-2-yl)-2-(trifluoromethyl)pyridine (S2f)

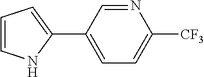 [SMILES: FC(F)(F)c1ccc(-c2ccc[nH]2)cn1]

Yield=92%

¹H NMR (CDCl₃, 400 MHz): δ=6.29-6.41 (m, 1H), 6.64-6.74 (m, 1H), 6.92-7.04 (m, 1H), 7.63 (d, J=8.2 Hz, 1H), 7.89 (dd, J=8.2, 1.8 Hz, 1H), 8.82 (d, J=1.8 Hz, 1H), 9.32 (br. s., 1H).

¹³C NMR (CDCl₃, 400 MHz): δ=109.2, 111.0, 120.9 (q, J=2.9 Hz), 121.8, 121.8 (q, J=273.7 Hz), 127.2, 131.5, 131.7, 144.7 (q, J=35.1 Hz), 144.9.

### Example 5.18 3-(1H-Pyrrol-2-yl)-6-(trifluoromethyl)pyridazine (S2g)

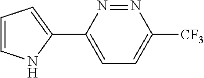 [SMILES: FC(F)(F)c1ccc(-c2ccc[nH]2)nn1]

Yield=95%

¹H NMR (DMSO-d₆, 400 MHz): δ=6.25-6.29 (m, 1H), 7.07-7.14 (m, 2H), 8.10 (d, J=9.0 Hz, 1H), 8.20 (d, J=9.0 Hz, 1H), 12.15 (br. s., 1H).

¹³C NMR (DMSO-d₆, 100 MHz): δ=110.5, 112.7, 122.0 (q, J=273.5 Hz), 122.9, 124.2, 124.8 (q, J=2.2 Hz), 126.9, 147.3 (q, J=33.7 Hz), 154.8.

### Example 5.19 5-Chloro-2-(1H-pyrrol-2-yl)pyrimidine (S2h)

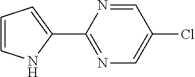 [SMILES: Clc1cnc(-c2ccc[nH]2)nc1]

Yield=90%.

¹H NMR (DMSO-d₆, 400 MHz): δ=6.20 (s, 1H), 6.93 (d, J=1.8 Hz, 1H), 6.97 (s, 1H), 8.78 (s, 2H), 11.77 (br. s., 1H).

¹³C NMR (DMSO-d₆, 100 MHz): δ=110.1, 112.3, 123.2, 125.7, 129.4, 155.6 (2C), 157.1.

### Example 5.20 General Procedure C: For Acylation

Crude pyrrole from the previous step (1 equiv) was dissolved in CH₂Cl₂ (0.5 M solution) and pyridine (1.2 equiv) was added followed by dropwise addition of TFAA (1.2 equiv). After completion of the addition, the mixture was stirred for 1 h and the solvent was evaporated. Product was triturated in water and precipitate was filtered, washed with water twice and dried on filter. Compound S3a-h were obtained following the general procedure C.

### Example 5.21 2,2,2-Trifluoro-1-(5-(5-methylpyridin-2-yl)-1H-pyrrol-2-yl)ethanone (S3a)

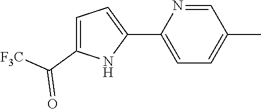 [SMILES: Cc1ccc(-c2ccc(C(=O)C(F)(F)F)[nH]2)nc1]

Yield=89%.

¹H NMR (DMSO-d₆, 400 MHz): δ=2.34 (s, 3H), 7.05 (d, J=4.2 Hz, 1H), 7.28 (dd, J=3.9, 1.9 Hz, 1H), 7.75 (dd, J=8.0, 1.4 Hz, 1H), 8.09 (d, J=8.1 Hz, 1H), 8.49-8.52 (m, 1H), 12.87 (br. s., 1H).

¹³C NMR (DMSO-d₆, 100 MHz): δ=17.8, 111.8, 117.0 (q, J=290.3 Hz), 120.6, 122.9 (q, J=3.5 Hz), 126.1, 133.4, 137.8, 142.6, 145.3, 149.8, 168.1 (q, J=35.0 Hz).

### Example 5.22 1-(5-(5-Chloropyridin-2-yl)-1H-pyrrol-2-yl)-2,2,2-trifluoroethanone (S3b)

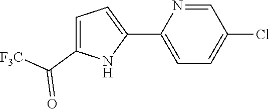 [SMILES: O=C(c1ccc(-c2ccc(Cl)cn2)[nH]1)C(F)(F)F]

Yield=94%.

¹H NMR (DMSO-d₆, 400 MHz): δ=7.09 (dd, 1H), 7.29 (dt, J=4.1, 2.0 Hz, 1H), 8.07 (dd, J=8.6, 2.5 Hz, 1H), 8.23 (d, J=8.6 Hz, 1H), 8.69 (d, J=2.4 Hz, 1H), 13.04 (br. s., 1H).

¹³C NMR (DMSO-d₆, 100 MHz): δ=112.5, 117.0 (q, J=290.25 Hz), 122.0, 122.7 (q, J=3.5 Hz), 126.6, 130.7, 137.0, 141.5, 146.7, 148.3, 168.5 (q, J=35.0 Hz).

### Example 5.23 2,2,2-Trifluoro-1-(5-(5-fluoropyridin-2-yl)-1H-pyrrol-2-yl)ethanone (S3c)

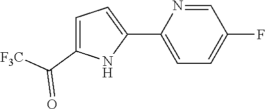 [SMILES: O=C(c1ccc(-c2ccc(F)cn2)[nH]1)C(F)(F)F]

Yield=91%.

¹H NMR (DMSO-d₆, 400 MHz): δ=7.04 (dd, J=4.1, 2.3 Hz, 1H), 7.28 (dt, J=4.1, 2.0 Hz, 1H), 7.87 (td, J=8.8, 2.9 Hz, 1H), 8.27 (dd, J=8.9, 4.4 Hz, 1H), 8.65 (d, J=2.8 Hz, 1H), 12.96 (br. s., 1H).

¹³C NMR (DMSO-d₆, 100 MHz): δ=112.0, 117.0 (q, J=289.9 Hz), 122.4 (d, J=4.8 Hz), 122.7 (q, J=3.5 Hz), 124.1 (d, J=19.0 Hz), 126.4, 138.0 (d, J=24.5 Hz), 141.8, 144.9 (d, J=3.9 Hz), 158.7 (d, J=256.2 Hz), 168.3 (q, J=35.0 Hz).

### Example 5.24 2,2,2-Trifluoro-1-(5-(5-(trifluoromethyl)pyridin-2-yl)-1H-pyrrol-2-yl)ethanone (S3d)

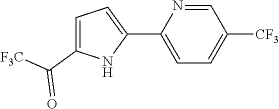 [SMILES: O=C(c1ccc(-c2ccc(C(F)(F)F)cn2)[nH]1)C(F)(F)F]

Yield=92%.

¹H NMR (DMSO-d₆, 400 MHz): δ=7.19 (dd, J=4.0, 2.2 Hz, 1H), 7.27-7.32 (m, 1H), 8.33 (dd, J=8.4, 1.7 Hz, 1H), 8.40 (d, J=8.3 Hz, 1H), 8.99 (s, 1H), 13.19 (br. s., 1H)

¹³C NMR (DMSO-d₆, 100 MHz): δ=113.7, 117.2 (q, J=289.8 Hz), 120.9, 122.7 (q, J=1.5 Hz), 124.0 (q, J=271.5 Hz), 124.5 (q, J=32.2 Hz), 127.5, 135.1, 141.2, 146.7, 152.0, 169.1 (q, J=34.4 Hz).

### Example 5.25 1-(5-(6-Chloropyridin-3-yl)-1H-pyrrol-2-yl)-2,2,2-trifluoroethanone (S3e)

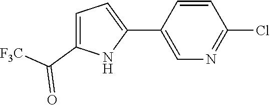 [SMILES: O=C(c1ccc(-c2ccc(Cl)nc2)[nH]1)C(F)(F)F]

Yield=87%.

¹H NMR (DMSO-d₆, 400 MHz): δ=7.08 (dd, J=4.2, 2.4 Hz, 1H), 7.33 (dt, J=4.1, 2.0 Hz, 1H), 7.64 (d, J=8.4 Hz, 1H), 8.43 (dd, J=8.4, 2.6 Hz, 1H), 9.02 (d, J=2.4 Hz, 1H), 13.14 (br. s., 1H).

¹³C NMR (DMSO-d₆, 100 MHz): δ=111.9, 117.1 (q, J=289.9 Hz), 123.2 (q, J=3.3 Hz), 124.5, 125.5, 126.8, 137.0, 139.1, 147.6, 150.2, 168.3 (q, J=35.0 Hz).

### Example 5.26 2,2,2-Trifluoro-1-(5-(6-(trifluoromethyl)pyridin-3-yl)-1H-pyrrol-2-yl)ethanone (S3f)

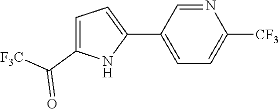 [SMILES: O=C(c1ccc(-c2ccc(C(F)(F)F)nc2)[nH]1)C(F)(F)F]

Yield=93%.

¹H NMR (DMSO-d₆, 400 MHz): δ=7.14 (d, J=4.3 Hz, 1H), 7.26-7.33 (m, 1H), 7.96 (d, J=8.3 Hz, 1H), 8.61 (dd, J=8.2, 1.8 Hz, 1H), 9.32 (d, J=1.7 Hz, 1H), 13.24 (br. s., 1H).

¹³C NMR (DMSO-d₆, 100 MHz): δ=112.6, 116.9 (q, J=289.8 Hz), 120.8 (q, J=2.2 Hz), 121.6 (q, J=273.7 Hz), 122.9 (q, J=3.7 Hz), 127.2, 129.1, 135.1, 138.4, 145.7 (q, J=34.4 Hz), 147.7, 168.5 (q, J=35.1 Hz).

### Example 5.27 2,2,2-Trifluoro-1-(5-(6-(trifluoromethyl)pyridazin-3-yl)-1H-pyrrol-2-yl)ethanone (S3g)

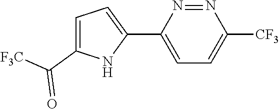 [SMILES: O=C(c1ccc(-c2ccc(C(F)(F)F)nn2)[nH]1)C(F)(F)F]

Yield=85%.

¹H NMR (DMSO-d₆, 400 MHz): δ=7.34-7.39 (m, 2H), 8.39 (d, J=9.0 Hz, 1H), 8.70 (d, J=9.0 Hz, 1H), 13.50 (br. s., 1H).

¹³C NMR (DMSO-d₆, 100 MHz): δ=114.1, 116.7 (q, J=289.8 Hz), 121.6 (q, J=274.0 Hz), 122.3 (q, J=3.1 Hz), 125.4 (q, J=2.0 Hz), 125.5, 128.0, 137.8, 149.2 (q, J=34.1 Hz), 153.8, 169.1 (q, J=35.4 Hz).

### Example 5.28 1-(5-(5-Chloropyrimidin-2-yl)-1H-pyrrol-2-yl)-2,2,2-trifluoroethanone (S3h)

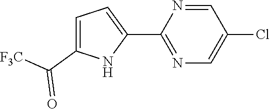 [SMILES: O=C(c1ccc(-c2ncc(Cl)cn2)[nH]1)C(F)(F)F]

Yield=86%%.

¹H NMR (DMSO-d₆, 400 MHz): δ=7.10 (d, J=3.9 Hz, 1H), 7.24 (s, 1H), 8.97 (s, 2H), 13.07 (br. s., 1H).

¹³C NMR (DMSO-d₆, 100 MHz): δ=114.2, 116.7 (q, J=289.8 Hz), 122.2 (q, J=2.9 Hz), 127.2, 129.2, 139.6, 155.3, 156.2 (2C), 168.7 (q, J=35.1 Hz).

### Example 5.29 General Procedure D: For Haloform Reaction

Appropriate trifluoroethanone (1 equiv) was added to a solution of NaOH (5 equiv) in dioxane-H₂O mixture (1:1, 0.5M solution). The resulting reaction mixture was refluxed for 20 h and cooled to the room temperature. A concentrated aqueous HCl solution (˜12 M, 5 equiv) was added dropwise. The resulting precipitate was filtered off, washed with H₂O and dried on filter. Compound S4a-h were obtained following the general procedure D.

### Example 5.30 5-(5-Methylpyridin-2-yl)-1H-pyrrole-2-carboxylic acid (S4a)

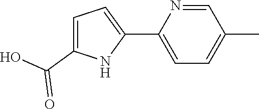

Yield=82%.

¹H NMR (DMSO-d₆, 400 MHz): δ=2.28 (s, 3H), 6.77-6.84 (m, 2H), 7.61 (dd, J=8.1, 2.1 Hz, 1H), 7.88 (d, J=8.1 Hz, 1H), 8.38 (d, J=1.8 Hz, 1H), 11.69 (br. s., 1H).

¹³C NMR (DMSO-d₆, 100 MHz): δ=17.9, 109.2, 116.5, 119.1, 124.8, 131.6, 136.4, 137.6, 147.1, 149.7, 162.0.

### Example 5.31 5-(5-Chloropyridin-2-yl)-1H-pyrrole-2-carboxylic acid (S4b)

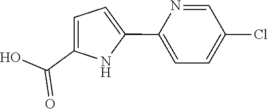 [SMILES: O=C(O)c1ccc(-c2ccc(Cl)cn2)[nH]1]

Yield=73%.

¹H NMR (DMSO-d₆, 400 MHz): δ=6.78-6.93 (m, 2H), 7.94 (dd, J=8.6, 2.4 Hz, 1H), 8.06 (d, J=8.6 Hz, 1H), 8.58 (d, J=2.2 Hz, 1H), 11.96 (br. s., 1H), 12.57 (br. s., 1H).

¹³C NMR (DMSO-d₆, 100 MHz): δ=110.3, 116.2, 120.3, 125.8, 128.8, 135.0, 136.8, 147.9, 148.2, 161.8.

### Example 5.32 5-(5-Fluoropyridin-2-yl)-1H-pyrrole-2-carboxylic acid (S4c)

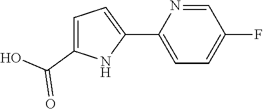 [SMILES: O=C(O)c1ccc(-c2ccc(F)cn2)[nH]1]

Yield=85%.

¹H NMR (DMSO-d₆, 400 MHz): δ=6.80-6.85 (m, 2H), 7.74 (td, J=8.8, 2.9 Hz, 1H), 8.08 (dd, J=8.8, 4.3 Hz, 1H), 8.53 (d, J=2.8 Hz, 1H), 11.86 (br. s., 1H), 12.52 (br. s., 1H).

¹³C NMR (DMSO-d₆, 100 MHz): δ=109.7, 116.3, 120.6 (d, J=4.4 Hz), 124.1 (d, J=18.8 Hz), 125.1, 135.4, 137.4 (d, J=24.0 Hz), 146.4 (d, J=3.7 Hz), 158.0 (d, J=253.4 Hz), 161.8.

### Example 5.33 5-(5-(Trifluoromethyl)pyridin-2-yl)-1H-pyrrole-2-carboxylic acid S4d)

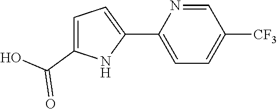 [SMILES: O=C(O)c1ccc(-c2ccc(C(F)(F)F)cn2)[nH]1]

Yield=80%.

¹H NMR (DMSO-d₆, 400 MHz): δ=6.76-6.95 (m, 1H), 6.95-7.08 (m, 1H), 8.12-8.31 (m, 2H), 8.89 (s, 1H), 12.18 (br. s., 1H), 12.71 (br. s., 1H).

13C NMR (DMSO-d₆, 100 MHz): δ=111.7, 116.3, 119.0, 122.7 (q, J=32.2 Hz), 124.0 (q, J=272.2 Hz), 126.7, 134.4 (q, J=3.7 Hz), 134.8, 146.2 (q, J=4.4 Hz), 153.1, 161.8.

### Example 5.34 5-(6-Chloropyridin-3-yl)-1H-pyrrole-2-carboxylic acid (S4e)

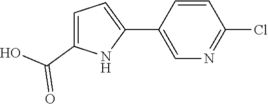 [SMILES: O=C(O)c1ccc(-c2ccc(Cl)nc2)[nH]1]

Yield=91%.

¹H NMR (DMSO-d₆, 400 MHz): δ=6.73-6.89 (m, 2H), 7.52 (d, J=8.4 Hz, 1H), 8.29 (dd, J=8.4, 2.6 Hz, 1H), 8.90 (d, J=2.4 Hz, 1H), 12.26 (br. s., 1H), 12.51 (br. s., 1H).

¹³C NMR (DMSO-d₆, 100 MHz): δ=109.2, 116.4, 124.2, 125.5, 127.1, 132.0, 135.7, 146.3, 148.2, 161.8.

### Example 5.35 5-(6-(Trifluoromethyl)pyridin-3-yl)-1H-pyrrole-2-carboxylic acid (S4f)

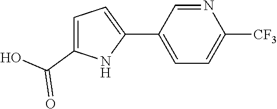 [SMILES: O=C(O)c1ccc(-c2ccc(C(F)(F)F)nc2)[nH]1]

Yield=80%.

¹H NMR (DMSO-d₆, 400 MHz): δ=6.84-6.87 (m, 1H), 6.87-6.90 (m, 1H), 7.85 (d, J=8.3 Hz, 1H), 8.45 (dd, J=8.3, 1.5 Hz, 1H), 9.20 (d, J=1.2 Hz, 1H), 12.40 (br. s., 1H).

¹³C NMR (DMSO-d₆, 100 MHz): δ=110.5, 116.6, 120.9 (q, J=2.9 Hz), 243.9 (q, J=273.7 Hz), 126.5, 130.8, 131.8, 133.5, 144.2 (q, J=33.7 Hz), 146.7, 161.9.

### Example 5.36 5-(6-(Trifluoromethyl)pyridazin-3-yl)-1H-pyrrole-2-carboxylic acid (S4g)

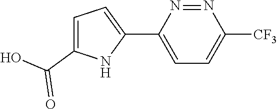 [SMILES: O=C(O)c1ccc(-c2ccc(C(F)(F)F)nn2)[nH]1]

Yield=87%.

¹H NMR (DMSO-d₆, 400 MHz): δ=6.91 (dd, J=3.9, 2.3 Hz, 1H), 7.20 (dd, J=3.9, 2.4 Hz, 1H), 8.27 (d, J=9.0 Hz, 1H), 8.57 (d, J=9.0 Hz, 1H), 12.55 (br. s., 1H), 12.83 (br. s., 1H).

¹³C NMR (DMSO-d₆, 100 MHz): δ=112.7, 116.4, 121.9 (q, J=273.7 Hz), 123.9, 125.1 (q, J=2.2 Hz), 127.9, 131.7, 148.2 (q, J=33.7 Hz), 154.7, 161.6.

### Example 5.37 5-(5-Chloropyrimidin-2-yl)-1H-pyrrole-2-carboxylic acid (S4h)

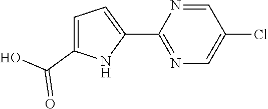 [SMILES: O=C(O)c1ccc(-c2ncc(Cl)cn2)[nH]1]

Yield=75%.

¹H NMR (DMSO-d₆, 400 MHz): δ=6.81 (d, J=3.7 Hz, 1H), 6.96 (d, J=3.7 Hz, 1H), 8.87 (s, 2H), 11.63 (br. s., 1H).

¹³C NMR (DMSO-d₆, 100 MHz): δ=112.7, 115.7, 127.4, 128.1, 132.9, 155.9 (2C), 156.2, 161.7.

### Example 5.38

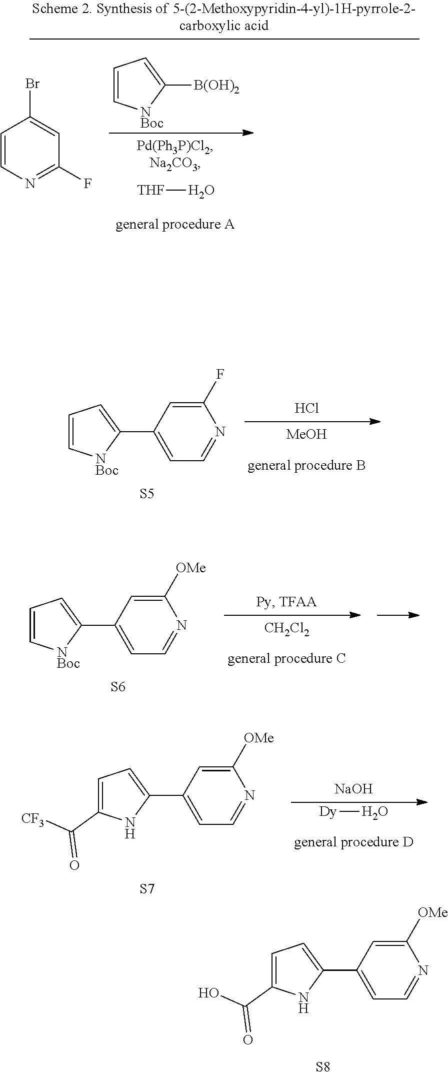

### Example 5.39 Tert-butyl 2-(2-fluoropyridin-4-yl)-1H-pyrrole-1-carboxylate (S5)

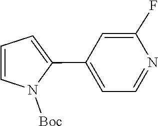 [SMILES: CC(C)(C)OC(=O)n1cccc1-c1ccnc(F)c1]

Compound S5 was obtained following the general procedure A. Yield=67%.

¹H NMR (CDCl₃, 400 MHz): δ=1.45 (s, 9H), 6.24-6.27 (m, 1H), 6.36 (dd, J=3.3, 1.7 Hz, 1H), 6.88-6.91 (m, 1H), 7.14-7.17 (m, 1H), 7.41 (dd, J=3.2, 1.7 Hz, 1H), 8.16 (d, J=5.2 Hz, 1H).

¹³C NMR (CDCl₃, 100 MHz): δ=27.7, 84.9, 109.0 (d, J=38.3 Hz), 111.2, 117.0, 121.6 (d, J=3.9 Hz), 124.8, 131.2 (d, J=3.9 Hz), 146.6 (d, J=15.5 Hz), 147.1 (d, J=8.9 Hz), 148.8, 163.6 (d, J=237.2 Hz).

### Example 5.40 2-Methoxy-4-(1H-pyrrol-2-yl)pyridine (S6)

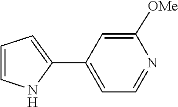 [SMILES: COc1cc(-c2ccc[nH]2)ccn1]

Compound S6 was obtained following the general procedure B. Yield 92%.

¹H NMR (CDCl₃, 400 MHz): δ=3.96 (s, 3H), 6.31-6.36 (m, 1H), 6.68-6.74 (m, 1H), 6.78 (d, J=1.2 Hz, 1H), 6.93 (s, 1H), 6.99 (dd, J=5.5, 1.5 Hz, 1H), 8.11 (d, J=5.5 Hz, 1H), 8.89 (br. s., 1H).

¹³C NMR (CDCl₃, 100 MHz): δ=53.7, 103.8, 109.0, 110.7, 112.3, 121.0, 129.3, 142.4, 147.3, 165.1.

### Example 5.41 2,2,2-Trifluoro-1-(5-(2-methoxypyridin-4-yl)-1H-pyrrol-2-yl)ethanone (S7)

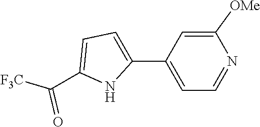 [SMILES: COc1cc(-c2ccc(C(=O)C(F)(F)F)[nH]2)ccn1]

Compound S7 was obtained following the general procedure C. Yield=78%

¹H NMR (DMSO-d₆, 400 MHz): δ=3.88 (s, 3H), 7.10 (dd, J=4.2, 2.4 Hz, 1H), 7.28 (dt, J=4.0, 1.9 Hz, 1H), 7.46 (s, 1H), 7.54 (dd, J=5.4, 1.4 Hz, 1H), 8.21 (d, J=5.4 Hz, 1H), 13.12 (br. s., 1H).

¹³C NMR (DMSO-d₆, 100 MHz): δ=53.4, 106.5, 112.4, 114.0, 116.9 (q, J=290.1 Hz), 122.8 (q, J=3.5 Hz), 126.8, 139.6, 140.2, 147.7, 164.5, 168.5 (q, J=35.0 Hz).

### Example 5.42 5-(2-Methoxypyridin-4-yl)-1H-pyrrole-2-carboxylic acid (S8)

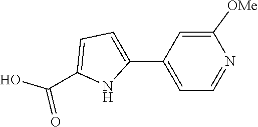 [SMILES: COc1cc(-c2ccc(C(=O)O)[nH]2)ccn1]

Compound S8 was obtained following the general procedure D. Yield=93%.

¹H NMR (DMSO-d₆, 400 MHz): δ=3.86 (s, 3H), 6.80-6.84 (m, 1H), 6.84-6.88 (m, 1H), 7.35 (s, 1H), 7.43 (dd, J=5.5, 1.4 Hz, 1H), 8.11 (d, J=5.5 Hz, 1H), 12.26 (br. s., 1H), 12.56 (br. s., 1H).

¹³C NMR (DMSO-d₆, 100 MHz): δ=53.2, 104.9, 110.2, 113.2, 116.2, 125.9, 133.5, 141.3, 147.2, 161.7, 164.5.

### Example 5.43 Synthesis of Methyl 2-((tert-butoxycarbonyl)amino)-3-iodobut-2-enoate (R4)

### Methyl 2-((tert-butoxycarbonyl)amino)but-2-enoate (R3)

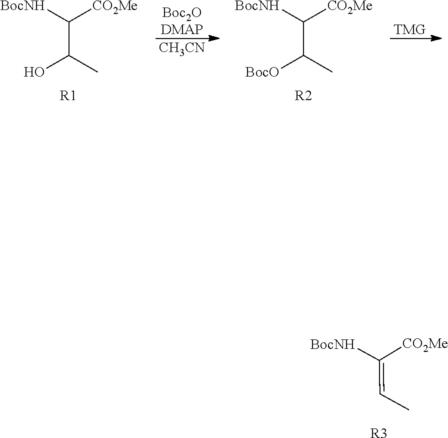

DMAP (3.64 g, 0.1 equiv.) was added to a solution of the N-Boc acid methyl ester R1 (69.59 g, 1 equiv.) in dry acetonitrile (300 mL), followed by di-tert-butyl dicarbonate (65.0 g, 1.0 equiv.) with rapid stirring at room temperature. The reaction was monitored by TLC (diethyl ether/n-hexane, 1:1) until all the reactant had been consumed. TMG (3.44 mL, 0.1 equiv) was then added, stirring was continued, and the reaction was followed by TLC. When all the reactant had been consumed, evaporation at reduced pressure gave a residue that was partitioned between CH₂Cl₂ (300 mL) and 5% HCl (200 mL). The organic phase was thoroughly washed with NaHCO₃ and dried with Na₂SO₄. Removal of the solvent afforded the corresponding N-Boc-dehydroamino acid methyl ester. M=63.88 g.

¹H NMR (CDCl₃, 400 MHz): 5=1.48 (s, 9H), 1.82 (d, J=7.2 Hz, 3H), 3.78 (s, 3H), 6.00 (br. s, 1H), 6.69 (q, J=7.0 Hz, 1H).

### Methyl 2-((tert-butoxycarbonyl)amino)-3-iodobut-2-enoate (R4)

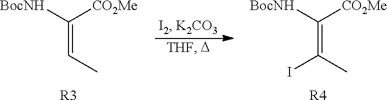

A flask was charged with the dehydroamino acid derivative R3 (63.88 g, 48.4 mmol), K₂CO₃ (81.7 g, 2 equiv.) and THF (400 mL). 12 (90.9 g, 1.2 equiv.) was added and the reaction mixture was heated at reflux for ˜4 h. After the system had cooled to room temperature, the reaction mixture was quenched with a 10% solution of Na₂SO₃ (100 mL). The mixture was extracted with CH₂Cl₂ (3×100 mL). The combined organic layers were dried over Na₂SO₄, filtered and evaporated. The residue was subjected to column chromatography (eluent hexanes/EtOAc, 10:1). M=52.2 g.

¹H NMR (CDCl₃, 400 MHz): 5=1.47 (s, 9H), 2.74 (s, 3H), 3.83 (s, 3H), 6.17 (br. s, 1H).

### Example 5.44

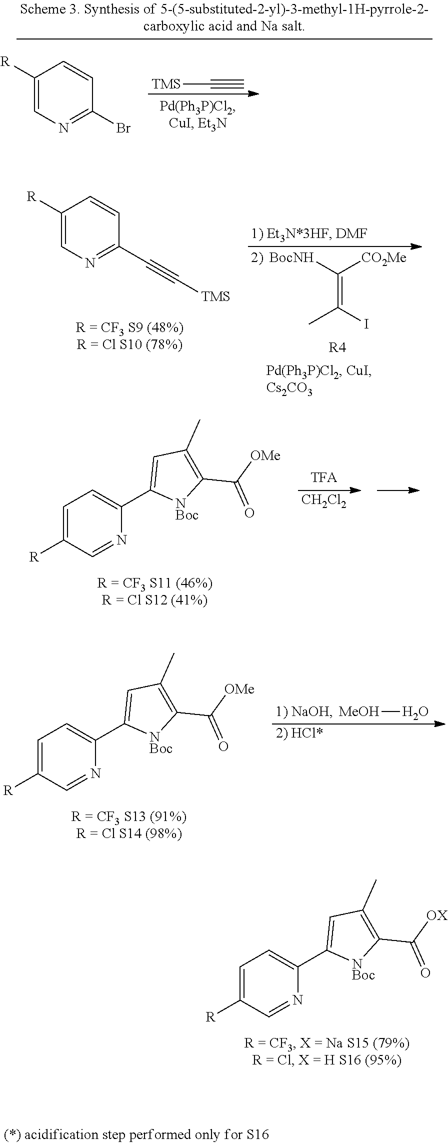

### Example 5.45 5-(Trifluoromethyl)-2-((trimethylsilyl)ethynyl)pyridine (S9)

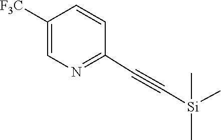 [SMILES: C[Si](C)(C)C#Cc1ccc(C(F)(F)F)cn1]

In a pressurized vessel equipped with magnetic stirring bar containing solution of 2-bromo-5-(trifluoromethyl)pyridine (30 g; 133 mmol, 1 equiv) in Et₃N (265 mL), TMS-acetylene (26.07 g, 36.8 mL, 265 mmol, 2 equiv.), Pd(Ph₃P)Cl₂ (0.93 g; 1 mol. %), CuI (0.51 g; 2 mol. %) were added under argon atmosphere. It was heated to 50-60° C. and the mixture was stirred at this temperature for 6-8 h (TLC-control). Water (500 ml) was added and extracted with hexane (3×150 mL). Combined extracts were washed with water and dried with anhydrous sodium sulfate. Solvent was removed by rotary evaporation and the residue was purified using column chromatography (eluent: Hexane-EtOAc, 30:1, Rf=0.5 in hexane-EtOAc 30:1). M=15.6 g. Yield=48%.

¹H NMR (CDCl₃, 400 MHz): δ=0.28 (s, 9H), 7.55 (d, J=8.2 Hz, 1H), 7.87 (dd, J=8.2, 2.2 Hz, 1H), 8.81 (s, 1H).

¹³C NMR (CDCl₃, 400 MHz): δ=−0.4 (3C), 98.2, 102.5, 123.3 (q, J=272.2 Hz), 125.6 (q, J=33.3 Hz), 126.9, 133.4 (q, J=3.1 Hz), 146.5, 146.8 (q, J=3.8 Hz).

### Example 5.46 5-Chloro-2-((trimethylsilyl)ethynyl)pyridine (S10)

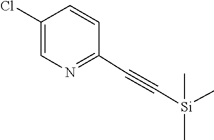

In a pressurized vessel equipped with magnetic stirring bar containing solution of 2-bromo-5-chloropyridine (20 g; 104 mmol, 1 equiv) in Et₃N (210 mL), TMS-acetylene (20.42 g, 28.8 mL, 208 mmol, 2 equiv.), Pd(Ph₃P)Cl₂ (0.73 g; 1 mol. %), CuI (0.4 g; 2 mol. %) were added under argon atmosphere. It was heated to 50-60° C. and the mixture was stirred at this temperature for 6-8 h (TLC-control). Water (500 ml) was added and extracted with hexane (3×150 mL). Combined extracts were washed with water and dried with anhydrous sodium sulfate. Solvent was removed by rotary evaporation and the residue was purified using column chromatography (eluent: Hexane-EtOAc, 30:1, Rf=0.4 in hexane-EtOAc 30:1). M=16.7 g. Yield=74%.

¹H NMR (CDCl₃, 400 MHz): δ=0.27 (s, 9H), 7.40 (d, J=8.4 Hz, 1H), 7.62 (dd, J=8.4, 2.4 Hz, 1H), 8.52 (d, J=2.4 Hz, 1H).

¹³C NMR (CDCl₃, 100 MHz): δ=−0.3 (3c), 96.1, 102.6, 127.8, 131.5, 135.9, 141.0, 148.9.

### Example 5.47 1-Tert-butyl 2-methyl 3-methyl-5-(5-(trifluoromethyl)pyridin-2-yl)-1H-pyrrole-1,2-dicarboxylate (S11)

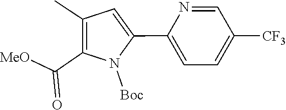 [SMILES: COC(=O)c1c(C)cc(-c2ccc(C(F)(F)F)cn2)n1C(=O)OC(C)(C)C]

To the solution of S9 (9.81 g, 40 mmol, 1 equiv) in DMF (200 mL), triethylamine trihydrofluoride (1.98 g, 2 mL, 12 mmol, 0.3 equiv) was added and mixture was stirred for 1 h under argon atmosphere. Then methyl (E)-2-(tert-butoxycarbonylamino)-3-iodo-but-2-enoate (13.75 g, 1 equiv.), Pd(Ph₃P)Cl₂ (1.42 g; 5 mol. %), CuI (0.77 g; 10 mol. %) and Cs₂CO₃ (26.27 g; 80 mmol, 2 equiv.) were added under argon atmosphere. It was heated to 70-80° C. and the mixture was stirred at this temperature for 10-15 h (TLC-control). Water (300 ml) was added and extracted with Et₂O (3×150 mL). Combined extracts were washed with water and brine, dried over Na₂SO₄, filtered, and concentrated. Purification by flash chromatography using hexane-EtOAc mixture (20:1) as eluent. M=7.1 g, Yield 46%.

¹H NMR (CDCl₃, 400 MHz): δ=1.64 (s, 9H), 2.34 (s, 3H), 3.89 (s, 3H), 6.52 (s, 1H), 7.64 (d, J=8.1 Hz, 1H), 7.89 (d, J=7.8 Hz, 1H), 8.73 (br. s., 1H).

¹³C NMR (CDCl₃, 400 MHz): δ=13.1, 27.4 (3C), 51.5, 85.1, 114.1, 120.5, 123.1, 123.6 (q, J=272.2 Hz), 124.3 (q, J=32.9 Hz), 129.2, 133.2, 133.6 (q, J=2.9 Hz), 145.1 (q, J=4.4 Hz), 149.9, 152.5, 161.2.

### Example 5.48 1-Tert-butyl 2-methyl 5-(5-chloropyridin-2-yl)-3-methyl-1H-pyrrole-1,2-dicarboxylate (S12)

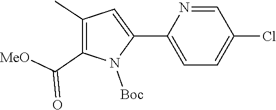 [SMILES: COC(=O)c1c(C)cc(-c2ccc(Cl)cn2)n1C(=O)OC(C)(C)C]

To the solution of S10 (13.1 g, 62 mmol, 1 equiv) in DMF (310 mL), triethylamine trihydrofluoride (3.01 g, 3.05 mL, 12 mmol, 0.3 equiv) was added and mixture was stirred for 1 h under argon atmosphere. Then methyl (E)-2-(tert-butoxycarbonylamino)-3-iodo-but-2-enoate (21.31 g, 1 equiv.), Pd(Ph₃P)Cl₂ (1.32 g; 3 mol. %), CuI (1.2 g; 10 mol. %) and Cs₂CO₃ (40.7 g; 125 mmol, 2 equiv.) were added under argon atmosphere. It was heated to 70-80° C. and the mixture was stirred at this temperature for 10-15 h (TLC-control). Water (300 ml) was added and extracted with Et₂O (3×150 mL). Combined extracts were washed with water and brine, dried over Na₂SO₄, filtered, and concentrated. M=8.95 g, Yield 41%.

¹H NMR (CDCl₃, 400 MHz): δ=1.61 (s, 9H), 2.33 (s, 3H), 3.87 (s, 3H), 6.41 (s, 1H), 7.49 (d, J=8.5 Hz, 1H), 7.65 (dd, J=8.5, 2.4 Hz, 1H), 8.45 (d, J=2.1 Hz, 1H).

¹³C NMR (CDCl₃, 100 MHz): δ=13.3, 27.5 (3C), 51.6, 85.1, 113.2, 122.0, 122.4, 129.5, 130.5, 133.9, 136.3, 147.3, 147.8, 150.1, 161.4.

### Example 5.49 Methyl 3-methyl-5-(5-(trifluoromethyl)pyridin-2-yl)-1H-pyrrole-2-carboxylate (S13)

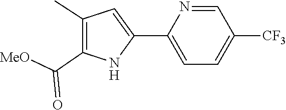 [SMILES: COC(=O)c1[nH]c(-c2ccc(C(F)(F)F)cn2)cc1C]

To a solution of S11 (5.87 g, 15 mmol, 1 equiv) in CH₂Cl₂ (50 ml) TFA (11 g, 7.4 mL, 76 mmol, 5 equiv) was added in one portion and mixture was stirred overnight. Then solvent was evaporated and 10% aqueous K₂CO₃ was added and mixture was extracted with CH₂Cl₂ (3×100 mL), dried over Na₂SO₄, filtered, and concentrated. Purification by flash chromatography using hexane-EtOAc mixture (10:1) as eluent. M=3.96 g. Yield 91%.

¹H NMR (CDCl₃, 400 MHz): δ=2.40 (s, 3H), 3.90 (s, 3H), 6.62 (d, J=2.8 Hz, 1H), 7.61 (d, J=8.3 Hz, 1H), 7.87 (dd, J=8.4, 2.1 Hz, 1H), 8.76 (s, 1H), 10.01 (br. s., 1H).

¹³C NMR (CDCl₃, 400 MHz): δ=12.8, 51.4, 112.1, 118.4, 121.5, 123.7 (q, J=272.2 Hz), 124.1 (q, J=32.9 Hz), 129.6, 132.4, 133.8 (q, J=3.7 Hz), 146.4 (q, J=4.4 Hz), 152.1, 161.6.

### Example 5.50 Methyl 5-(5-chloropyridin-2-yl)-3-methyl-1H-pyrrole-2-carboxylate (S14)

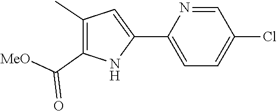 [SMILES: COC(=O)c1[nH]c(-c2ccc(Cl)cn2)cc1C]

To a solution of S12 (8.25 g, 24 mmol, 1 equiv) in CH₂Cl₂ (100 ml) TFA (13.4 g, 9.0 mL, 118 mmol, 5 equiv) was added in a one portion and mixture was stirred overnight. Then solvent was evaporated and 10% aqueous K₂CO₃ was added and mixture was extracted with CH₂Cl₂ (3×100 mL), dried over Na₂SO₄, filtered, and concentrated. Purification by flash chromatography using hexane-EtOAc mixture (10:1) as eluent. M=5.76 g. Yield 98%.

¹H NMR (CDCl₃, 400 MHz): δ=2.38 (s, 3H), 3.88 (s, 3H), 6.50 (d, J=2.6 Hz, 1H), 7.46 (d, J=8.4 Hz, 1H), 7.62 (dd, J=8.5, 2.4 Hz, 1H), 8.45 (d, J=2.2 Hz, 1H), 9.89 (br. s., 1H).

¹³C NMR (CDCl₃, 100 MHz): δ=12.9, 51.4, 110.9, 119.8, 120.7, 129.7, 129.9, 132.7, 136.5, 147.4, 148.2, 161.7.

### Example 5.51 Sodium 3-methyl-5-(5-(trifluoromethyl)pyridin-2-yl)-1H-pyrrole-2-carboxylate (S15)

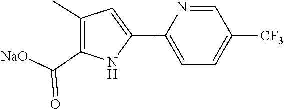

To a solution of S13 (3.96 g, 14 mmol, 1 equiv) in mixture of dioxane-H₂O (1:1, 30 mL), NaOH (0.61 g, 15 mmol, 1.1 equiv) was added in one portion and reaction mixture was stirred at reflux for 10-12 h (TLC-control). Mixture was evaporated to a volume of 10-20 mL, precipitate was filtered, washed with ether (2×50 mL) and dried under reduced pressure. M=3.21 g. Yield 79%.

¹H NMR (DMSO-d₆, 400 MHz): δ=2.28 (s, 3H), 6.72 (s, 1H), 7.92 (d, J=8.6 Hz, 1H), 8.01 (d, J=8.6 Hz, 1H), 8.75 (s, 1H), 10.34 (br. s., 1H).

¹³C NMR (DMSO-d₆, 100 MHz): δ=12.8, 113.1, 118.0, 121.0 (q, J=32.2 Hz), 122.5, 124.2 (q, J=271.5 Hz), 127.8, 131.3, 133.7 (q, J=2.2 Hz), 145.8 (q, J=3.7 Hz), 153.9, 167.1.

### Example 5.52 5-(5-Chloropyridin-2-yl)-3-methyl-1H-pyrrole-2-carboxylic acid (S16)

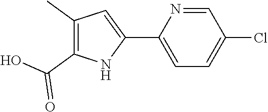 [SMILES: Cc1cc(-c2ccc(Cl)cn2)[nH]c1C(=O)O]

To a solution of S14 (5.7 g, 22 mmol, 1 equiv) in mixture of dioxane-H₂O (1:1, 60 mL), NaOH (1.06 g, 25 mmol, 1.1 equiv) was added in one portion and reaction mixture was stirred at reflux for 10-12 h (TLC-control). Then sodium-salt solution was acidified by addition of equivalent amount of HCl (12M, 2.08 mL, 1.1 equiv.) and precipitate was filtered. M=5.1 g. Yield 95%.

¹H NMR (DMSO-d₆, 400 MHz): δ=2.29 (s, 3H), 6.70 (s, 1H), 7.89 (d, J=8.3 Hz, 1H), 7.98 (d, J=8.4 Hz, 1H), 8.53 (s, 1H), 11.28 (br. s., 1H).

¹³C NMR (DMSO-d₆, 100 MHz): δ=12.8, 112.2, 120.2, 123.1, 126.9, 128.5, 132.2, 136.7, 147.8, 148.2, 163.1.

### Example 5.53

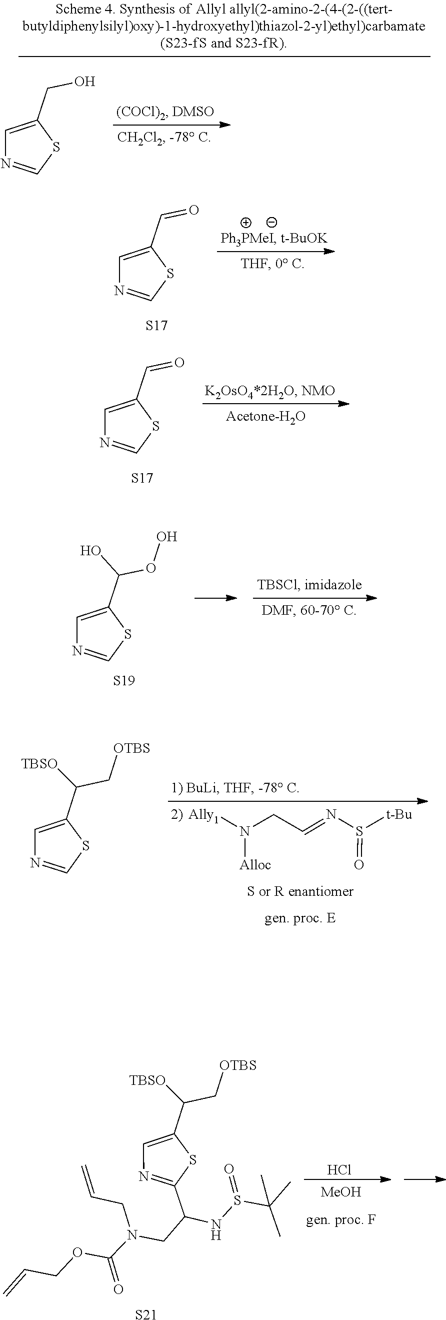

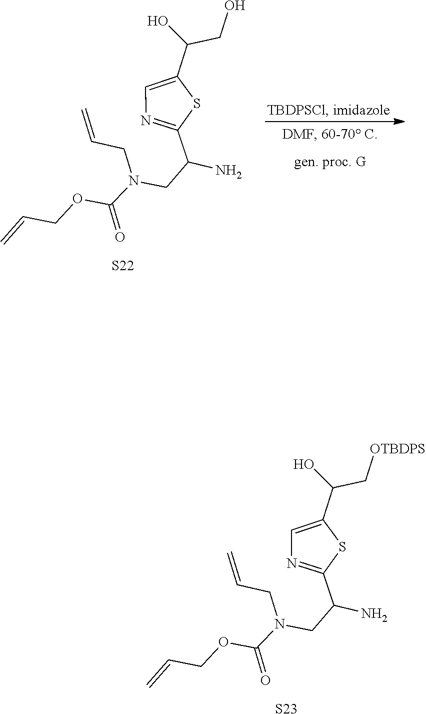

Note all enantiomer compounds derived from the stereo-controlled addition of the allyl N-allyl-N-[(2E)-2-tert-butylsulfinyliminoethyl]carbamate with absolute configuration S, have been specified with the fS descriptor; and all enantiomer compounds derived from the stereo-controlled addition of the allyl N-allyl-N-[(2E)-2-tert-butylsulfinyliminoethyl]carbamate with the absolute configuration R have been specified with the fR descriptor as per previous works [Eur J Med Chem. 2018 Jun. 25; 154:367-391. doi: 10.1016/j.ejmech.2018.04.062. Epub 2018 May 12.]

### Example 5.54 Thiazole-5-carbaldehyde (S17)

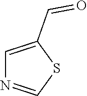

A solution of DMSO (16.96 g, 15.4 mL, 217 mmol, 2.5 equiv) in CH₂Cl₂ (100 mL) was added dropwise to a solution of oxalyl chloride (13.23 g, 8.94 mL, 104 mmol, 1.2 equiv) in CH₂Cl₂ (100 mL) at −70 to −80° C. The resulting solution was stirred for 10 minutes and a solution of alcohol (10 g, 87 mmol, 1 equiv) in CH₂Cl₂ (100 mL) was added dropwise at the same temperature. After 15 min, Et₃N (35.15 g, 48.3 mL, 347 mmol, 4 equiv) was added dropwise, and 5 minutes later the reaction mixture was allowed to warm to r.t. The reaction mixture was quenched with water (300 mL) and the layers were separated. The organic layer was dried over Na₂SO₄, filtered and concentrated in vacuo (bath temperature not exceeding 45-50° C.). The purification by flash chromatography (hexanes/EtOAc, 3:1) afforded aldehyde as a slightly brown oil. M=7.05 g. Yield=72%.

¹H NMR (CDCl₃, 400 MHz): δ=8.48 (s, 1H), 9.07 (s, 1H), 10.04 (d, J=1.0 Hz, 1H).

¹³C NMR (CDCl₃, 100 MHz): δ=139.4, 151.6, 160.1, 182.3.

### Example 5.55 5-Vinylthiazole (S18)

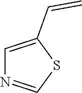

To the suspension of Ph₃P⁺MeI⁻ (25.74 g, 64 mmol, 1.1 equiv) in THF (70 ml), tBuOK (7.16 g, 64 mmol, 1.1 equiv) was added in a one portion. Mixture was refluxed for 1 h and cooled to the room temperature. Aldehyde S17 (6.55 g, 58 mmol, 1 equiv) in THF (50 ml) was added dropwise under cooling with water bath. Solvent was evaporated (bath temperature not exceeding 35° C.) and residue was triturated in ether and filtered. Filtrate was evaporated and crude product was purified by flash chromatography using hexane-EtOAc mixture (3:1) as eluent (Rf=0.5 in hexane-EtOAc 3:1). M=4.79 g. Yield=74%.

¹H NMR (CDCl₃, 400 MHz): δ=5.29 (d, J=10.9 Hz, 1H), 5.57 (d, J=17.3 Hz, 1H), 6.82 (dd, J=17.3, 10.9 Hz, 1H), 7.74 (s, 1H), 8.63 (s, 1H).

¹³C NMR (CDCl₃, 100 MHz): δ=117.2, 126.5, 138.0, 141.6, 151.7.

### Example 5.56 1-(Thiazol-5-yl)ethane-1,2-diol (S19)

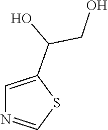

To a solution of alkene S18 (4.7 g, 42 mmol, 1 equiv) in acetone-water (4:1, 50 mL), NMO monohydrate (6.29 g, 47 mmol, 1.1 equiv.) and potassium osmate (VI) dehydrate (0.16 g, 1 mol. %) were added in a one portion. Mixture was refluxed for 10-12 h (TLC-control) and cooled to the room temperature. Solvent was evaporated and crude product was purified by flash chromatography using pure EtOAc as eluent (Rf=0.2 in EtOAc). M=2.83 g. Yield=46%.

¹H NMR (DMSO-d₆, 400 MHz): δ=3.44-3.50 (m, 1H), 3.51-3.58 (m, 1H), 4.85 (q, J=5.6 Hz, 1H), 5.00 (t, J=5.8 Hz, 1H), 5.80 (d, J=4.7 Hz, 1H), 7.77 (s, 1H), 8.96 (s, 1H).

¹³C NMR (DMSO-d₆, 100 MHz): δ=.

### Example 5.57 5-(2,2,3,3,8,8,9,9-Octamethyl-4,7-dioxa-3,8-disiladecan-5-yl)thiazole (S20)

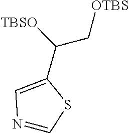 [SMILES: CC(C)(C)[Si](C)(C)OCC(O[Si](C)(C)C(C)(C)C)c1cncs1]

To a solution of alcohol S19 (2.83 g, 19 mmol, 1 equiv.) DMF (50 mL), imidazole (5.31 g, 78 mmol, 4 equiv) was added in one portion, followed by portionwise addition of TBSCl (8.81 g, 58 mmol, 3 equiv). The reaction mixture was stirred overnight at 50-60° C., cooled to the room temperature, diluted with water (100 ml), and extracted with EtoAc (3×50 mL). The combined organic phases were washed with water (50 mL), brine (50 mL) and dried over anhydrous Na₂SO₄, filtered and evaporated to give an oil, which was purified by flash chromatography using Hexane-EtOAc (10:1) as eluent (Rf=0.3 in 10:1 Hexane-EtOAc). M=5.87 g. Yield=80%.

¹H NMR (CDCl₃, 400 MHz): δ=−0.02-0.03 (m, 9H), 0.10 (s, 3H), 0.87 (s, 9H), 0.89 (s, 9H), 3.58 (dd, J=9.9, 6.3 Hz, 1H), 3.75 (dd, J=9.9, 5.9 Hz, 1H), 5.01 (t, J=6.1 Hz, 1H), 7.76 (s, 1H), 8.73 (s, 1H).

¹³C NMR (CDCl₃, 100 MHz): δ=−5.4, −5.4, −4.9, −4.7, 18.3, 18.5, 25.8 (3C), 26.0 (3C), 69.2, 70.4, 139.6, 141.7, 152.6.

### Example 5.58 General Procedure E: for 1,2-Addition

The appropriate thiazole (1.3 equiv) was dissolved in THF (1 M) and cooled to −78° C. At this temperature, n-BuLi (2.5 M, 1.4 equiv.) was added dropwise under a nitrogen atmosphere. The reaction mixture was stirred for 20 min at −78° C., and appropriate imine (1 equiv) was added dropwise as a solution in THF (1M). The reaction mixture was slowly (˜1 h) warmed to 0° C. and poured into water (5 mL per 1 g thiazole). The biphasic mixture was extracted with CH₂Cl₂ (3×100 mL). The combined organic phases were dried over Na₂SO₄, filtered, and evaporated to give a brown oil which was purified by column chromatography. Eluent: hexanes/EtOAc (10:1, 5:1, 1:1, 0:1).

### Example 5.59 General Procedure F: for Amine Deprotection

A 1 M HCl-MeOH solution was prepared by dropwise addition of AcCl (1.5 equiv) to a MeOH. The resulting solution was cooled to an ambient temperature and added to a flask containing appropriately protected compound (1 equiv). After dissolution, the reaction mixture was stirred for 1 h, evaporated, dissolved in CH₂Cl₂, and washed with 10% aqueous K₂CO₃. The organic layer was dried over Na₂SO₄, filtered, and evaporated and loaded on silica. Eluting with CH₂Cl₂/MeOH (50:1) provided pure amine as a yellow oil.

### Example 5.60 General Procedure G: For TBDPS-Protection

Diol (1 equiv.) was dissolved in CH₂Cl₂ (10 mL per 1 g), and imidazole (1.2 equiv) was added in one portion, followed by portionwise addition of TBDPSCl (1.1 equiv). The reaction mixture was stirred overnight, diluted with water (10 ml per 1 g), and extracted with CH₂Cl₂ (3×50 mL). The combined organic phases were washed with brine and dried over anhydrous Na₂SO₄, filtered, and evaporated to give an oil, which was purified by flash chromatography using Hexane-EtOAc mixture (1:1 and 0:1) as eluent.

### Example 5.61 Allyl allyl(2-amino-2-(4-(2-((tert-butyldiphenylsilyl)oxy)-1-hydroxyethyl)thiazol-2-yl)ethyl)carbamate (S23-fS and S23-fR)

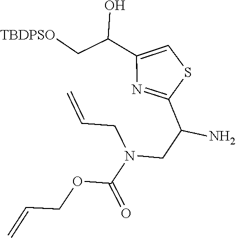

Compounds S23-fS and S23-fR were obtained following in succession the general procedure E, F and G from S20 (compounds S21, S22, were considered pure enough and used directly in the next steps without any further purifications)

S23-fS: M=2.94 g. Yield (over three steps)=30%.

S23-fR: M=1.81 g. Yield (over three steps)=27%.

¹H NMR (CDCl₃, 400 MHz): δ=1.08 (s, 9H), 2.43 (br. s., 3H), 3.51-4.00 (m, 6H), 4.40-4.54 (m, 1H), 4.60 (d, J=4.2 Hz, 2H), 5.03 (dd, J=7.1, 4.1 Hz, 1H), 5.07-5.17 (m, 2H), 5.20 (dd, J=10.5, 1.3 Hz, 1H), 5.29 (dd, J=17.2, 1.4 Hz, 1H), 5.67-5.83 (m, 1H), 5.85-5.98 (m, 1H), 7.36-7.49 (m, 6H), 7.55 (s, 1H), 7.61-7.68 (m, 4H).

### Example 5.62

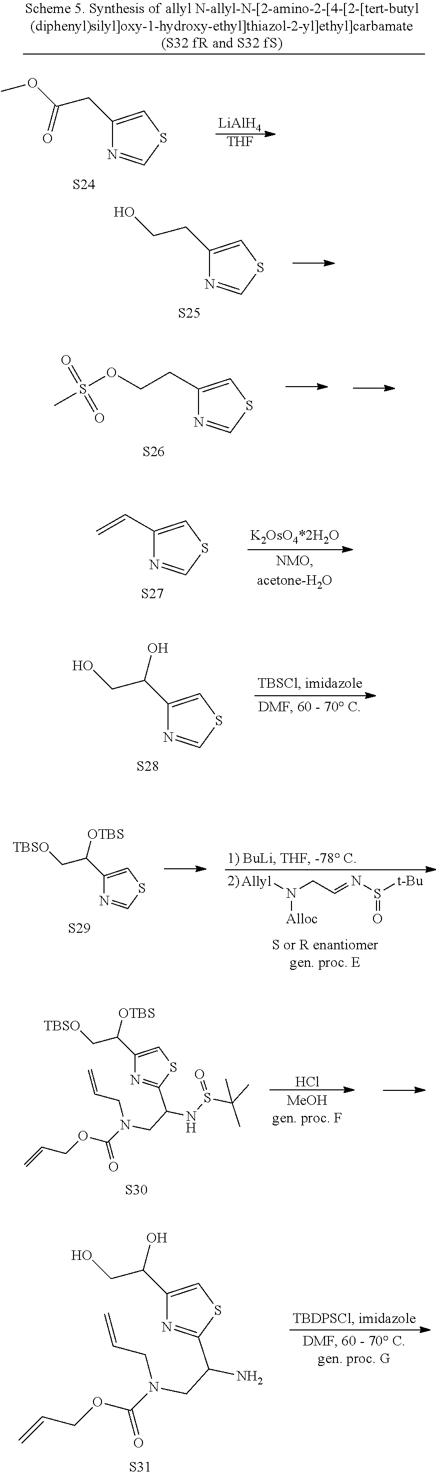

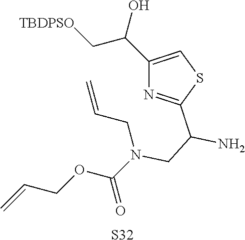

### Example 5.63 Methyl 2-(thiazol-4-yl)acetate (S24)

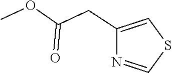

The compound (S24) was prepared by following a published procedure [Praveen, A. S., Yathirajan, H. S., Narayana, B. et al. Med Chem Res (2014) 23: 259. https://doi.org/10.1007/s00044-013-0629-x].

¹H NMR (CDCl₃, 400 MHz): δ=3.72 (s, 3H), 3.89 (s, 2H), 7.24 (d, J=1.8 Hz, 1H), 8.76 (d, J=1.9 Hz, 1H).

¹³C NMR (CDCl₃, 100 MHz): δ=36.6, 52.2, 116.1, 149.5, 152.8, 170.7.

### Example 5.64 2-(Thiazol-4-yl)ethanol (S25)

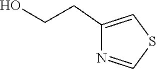

A solution of ester S24 (0.481 mol) in THF (480 mL) was added dropwise to a suspension of LiAlH4 (0.500 mmol, 1.0 equiv) in THF (480 mL) at 0° C. The reaction mixture was stirred for 60 min at 0° C. It was then quenched by successive addition of EtOAc (100 mL), water (37 mL), 10% NaOH (37 mL) solution, and water (74 mL) (the temperature should not exceed 0° C.). The precipitate was filtered and washed several times with THF. The filtrate was evaporated to give S25, which was used without further purifications.

¹H NMR (CDCl₃, 400 MHz): δ=3.03 (t, J=5.9 Hz, 2H), 3.43 (br. s., 1H), 3.93 (t, J=5.9 Hz, 2H), 7.04-7.05 (m, 1H), 8.74 (d, J=2.0 Hz, 1H).

¹³C NMR (CDCl₃, 100 MHz): δ=33.9, 61.5, 114.1, 152.8, 155.2.

### Example 5.65 2-(Thiazol-4-yl)ethyl methanesulfonate (S26)

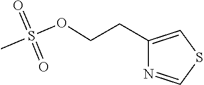

To a solution of corresponding alcohol S25 (2.14 mmol) and triethylamine (0.36 mL, 2.59 mmol) in anhydrous dichloromethane (6 mL), kept to 0° C., under an inert nitrogen atmosphere, was added mesyl chloride (2.31 mmol). The reaction was maintained at 0° C. during the first hour, followed by warming to room temperature, under vigorous stirring and nitrogen atmosphere for 3 h. Then, the solution was extracted with dichloromethane (3×30 mL) and the combined organic extracts were washed with a 10% aqueous HCl solution, brine, dried over Na₂SO₄, filtered and evaporated. The obtained residue was separated by chromatography on silica gel eluted with dichloromethane, followed by chloroform to yield the desired O-mesylated derivative.

Yield: 78%.

¹H NMR (CDCl₃, 400 MHz): δ=2.89 (s, 3H), 3.24 (t, J=6.5 Hz, 2H), 4.56 (t, J=6.5 Hz, 2H), 7.13 (d, J=1.3 Hz, 1H), 8.76 (d, J=1.8 Hz, 1H).

### Example 5.66 4-Vinylthiazole (S27)

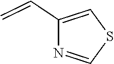

To a mixture of S26 (7.41 mmol) and TEA (5 mL) in DCM (50 mL) was added DBU (5 mL) slowly at 0° C. The mixture was stirred at r.t overnight, and then diluted with 50 mL of DCM, washed with 2N HCl×3 times and ×1 brine. The organic layer was dried over Na₂SO₄, filtered and concentrated to dryness. The residue was purified by prep-TLC to give S27. Yield: 58%.

¹H NMR (CDCl₃, 400 MHz): δ=5.39 (dd, J=10.9, 1.5 Hz, 1H), 6.09 (dd, J=17.3, 1.5 Hz, 1H), 6.77 (dd, J=17.3, 10.9 Hz, 1H), 7.14 (d, J=1.8 Hz, 1H), 8.76 (d, J=1.8 Hz, 1H).

¹³C NMR (CDCl₃, 100 MHz): δ=114.9, 116.9, 129.5, 152.9, 155.1.

### Example 5.67 1-(Thiazol-4-yl)ethane-1,2-diol (S28)

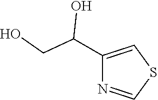

To a solution of alkene S27 (42 mmol, 1 equiv) in acetone-water (4:1, 50 mL), NMO monohydrate (47 mmol, 1.1 equiv.) and potassium osmate (VI) dehydrate (0.16 g, 1 mol. %) were added in a one portion. The mixture was refluxed for 10-12 h (TLC-control) and cooled to the room temperature. The solvent was evaporated and the crude product was purified by flash chromatography (Eluent: EtOAc). Yield 70%.

¹H NMR (CDCl₃, 400 MHz): δ=3.77 (dd, J=11.4, 7.2 Hz, 1H), 3.91 (dd, J=11.5, 3.4 Hz, 1H), 4.72 (br. s., 1H), 4.99 (dd, J=6.9, 3.3 Hz, 1H), 5.25 (br. s., 1H), 7.32 (d, J=1.9 Hz, 1H), 8.71 (d, J=2.0 Hz, 1H).

¹³C NMR (CDCl₃, 100 MHz): δ=66.4, 71.3, 115.2, 153.6, 157.5.

### Example 5.68 4-(2,2,3,3,8,8,9,9-octamethyl-4,7-dioxa-3,8-disiladecan-5-yl)thiazole (S29)

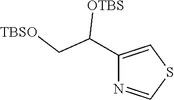 [SMILES: CC(C)(C)[Si](C)(C)OCC(O[Si](C)(C)C(C)(C)C)c1cscn1]

To a solution of alcohol S28 (19 mmol, 1 equiv.) DMF (50 mL), imidazole (5.31 g, 78 mmol, 4 equiv) was added in one portion, followed by portionwise addition of TBSCI (8.81 g, 58 mmol, 3 equiv). The reaction mixture was stirred overnight at 50-60° C., cooled to the room temperature, diluted with water (100 mL), and extracted with EtoAc (3×50 mL). The combined organic phases were washed with water (50 mL), brine (50 mL) and dried over anhydrous Na₂SO₄, filtered and evaporated to give an oil, which was purified by flash chromatography using Hexane-EtOAc (10:1) as eluent. Yield=78%.

¹H NMR (CDCl₃, 400 MHz): δ=−0.01-0.04 (m, 9H), 0.11 (s, 3H), 0.86 (s, 9H), 0.90 (s, 9H), 3.68 (dd, J=10.2, 7.2 Hz, 1H), 3.93 (dd, J=10.2, 3.8 Hz, 1H), 5.03 (dd, J=7.0, 3.6 Hz, 1H), 7.28 (d, J=1.9 Hz, 1H), 8.75 (d, J=2.1 Hz, 1H).

¹³C NMR (CDCl₃, 100 MHz): δ=−5.3, −5.2, −4.8, −4.5, 18.4, 18.5, 26.0 (3C), 26.1 (3C), 68.5, 73.8, 114.8, 152.3, 159.3.

### Example 5.69 Allyl N-allyl-N-[2-[4-[1,2-bis[[tert-butyl(dimethyl)silyl]oxy]ethyl]thiazol-2-yl]-2-(tert-butylsulfinylamino)ethyl]carbamate (S30)

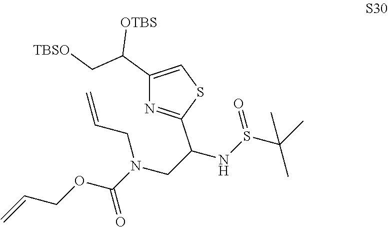 [SMILES: C=CCOC(=O)N(CC=C)CC(NS(=O)C(C)(C)C)c1nc(C(CO[Si](C)(C)C(C)(C)C)O[Si](C)(C)C(C)(C)C)cs1]

Compounds S30-fR and S30-fS were obtained following the general procedure E.

### Example 5.70 Allyl N-allyl-N-[2-amino-2-[4-(1,2-dihydroxyethyl)thiazol-2-yl]ethyl]carbamate (S31)

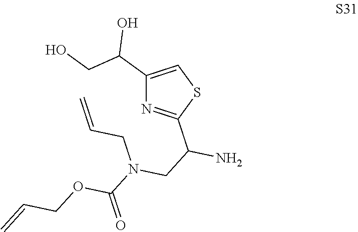 [SMILES: C=CCOC(=O)N(CC=C)CC(N)c1nc(C(O)CO)cs1]

Compounds S31-fR and S31-fS were obtained following the general procedure F.

### Example 5.71 Allyl N-allyl-N-[2-amino-2-[4-[2-[tert-butyl(diphenyl)silyl]oxy-1-hydroxy-ethyl]thiazol-2-yl]ethyl]carbamate (S32)

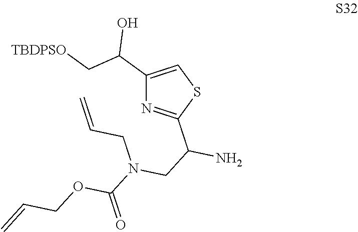

Compounds S32-fR and S32-fS were obtained following the general procedure G.

### Example 5.72

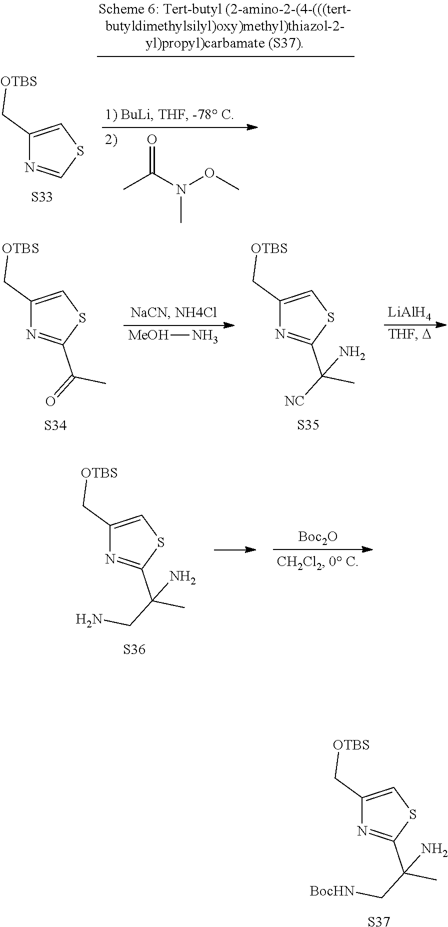

Synthesis of compound S33 was reported earlier (Eur J Med Chem. 2018 Jun. 25, 154:367-391. doi: 10.1016/j.ejmech.2018.04.062. Epub 2018 May 12.)

### Example 5.73 1-(4-(((Tert-butyldimethylsilyl)oxy)methyl)thiazol-2-yl)ethanone (S34)

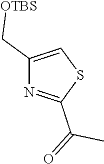 [SMILES: CC(=O)c1nc(CO[Si](C)(C)C(C)(C)C)cs1]

To a solution of thiazole S33 (49.06 g, 214 mmol, 1 equiv) in THF (210 mL), BuLi (2.5M in hexane, 85.55 mL, 1.2 equiv) was added dropwise at −78° C. under argon atmosphere. After the end of addition, mixture was stirred for 10 min, then solution of N-methoxy-N-methyl-acetamide (24.26 g, 235 mmol, 1.1 equiv) in THF (50 mL) was added dropwise. The resulting mixture was stirred overnight and poured into saturated solution of NH₄Cl (400 mL). The organic layer was separated and the aqueous solution was extracted with CH₂Cl₂ (2×100 mL). The combined organic layers were washed with brine (200 mL), dried over Na₂SO₄, filtered and concentrated. The crude product was purified by flash chromatography using hexane-EtOAc (10:1) as eluent. M=44.39 g. Yield=76%.

¹H NMR: (CDCl₃, 400 MHz) δ=0.12 (s, 6H), 0.94 (s, 9H), 2.67 (s, 3H), 4.90 (d, J=1.1 Hz, 2H), 7.54 (t, J=1.1 Hz, 1H).

¹³C NMR: (CDCl₃, 100 MHz) δ=−5.3 (2C), 18.5, 26.0 (3C), 26.1, 62.2, 121.5, 159.7, 166.8, 191.8.

### Example 5.74 2-Amino-2-(4-(((tert-butyldimethylsilyl)oxy)methyl)thiazol-2-yl)propanenitrile (S35)

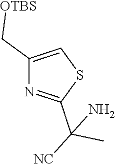 [SMILES: CC(N)(C#N)c1nc(CO[Si](C)(C)C(C)(C)C)cs1]

To the solution of S34 (44.39 g, 164 mmol, 1 equiv) in MeOH—NH₃ (330 mL), NaCN (16.0 g, 327 mmol, 2 equiv) and NH₄Cl (35.0 g, 654 mmol, 4 equiv) were added. Mixture was stirred for 4-5 days, then water (600 ml) was added and extracted with DCM (3×200 mL). Combined organic layers were washed with brine (200 mL) and dried over Na₂SO₄, filtered, and concentrated. The obtained crude product was purified by flash chromatography using hexane-EtOAc mixture (3:1) as eluent. Compound was obtained in racemic form. M=16.46 g. Yield=34%.

¹H NMR: (CDCl₃, 400 MHz) δ=0.12 (s, 6H), 0.95 (s, 9H), 1.92 (s, 3H), 2.42 (br. s., 2H), 4.86 (d, J=1.2 Hz, 2H), 7.21 (t, J=1.2 Hz, 1H).

¹³C NMR: (CDCl₃, 100 MHz) δ=−5.3 (2C), 18.4, 26.0 (3C), 30.3, 52.6, 62.3, 114.8, 121.9, 158.2, 170.6.

### Example 5.75 2-(4-(((Tert-butyldimethylsilyl)oxy)methyl)thiazol-2-yl)propane-1,2-diamine (S36)

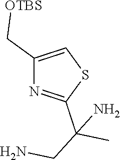 [SMILES: CC(N)(CN)c1nc(CO[Si](C)(C)C(C)(C)C)cs1]

A solution of S35 (16.46 g, 55 mmol, 1 equiv) in Et₂O (55 mL) was added dropwise to a suspension of LiAlH₄ (6.31 g, 166 mmol, 3 equiv.) in Et₂O (55 mL) at −10° C. The reaction mixture was stirred for 12 h at 0° C. It was then quenched by successive addition of water (7 mL), 10% NaOH (7 mL) solution, and water (7 mL) (the temperature should not exceed 0° C.). The precipitate was filtered and washed several times with Et₂O.

The filtrate was evaporated to give diamine S36, which was purified by flash chromatography using pure EtOAc and CHCl₃-MeOH saturated with NH₃ (10:1) as eluents). M=9.13 g. Yield=55%.

¹H NMR: (CDCl₃, 400 MHz) δ=0.09 (s, 6H), 0.92 (s, 9H), 1.46 (s, 3H), 1.61 (br. s., 4H), 2.76 (d, J=12.8 Hz, 1H), 3.15 (d, J=12.8 Hz, 1H), 4.81 (d, J=1.2 Hz, 2H), 7.06 (t, J=1.2 Hz, 1H).

¹³C NMR: (CDCl₃, 100 MHz) δ=−5.2 (2C), 18.5, 26.0 (3C), 28.1, 53.9, 58.1, 62.5, 113.5, 157.3, 179.8.

### Example 5.76 Tert-butyl (2-amino-2-(4-(((tert-butyldimethylsilyl)oxy)methyl)thiazol-2-yl)propyl)carbamate (S37)

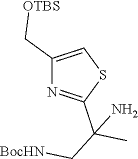 [SMILES: CC(C)(C)OC(=O)NCC(C)(N)c1nc(CO[Si](C)(C)C(C)(C)C)cs1]

To a solution of diamine S36 (9.13 g, 30 mmol, 1 equiv) in CH₂Cl₂ (300 ml), a solution of Boc₂O (6.93 g, 32 mmol, 1.05 equiv.) in CH₂Cl₂ (300 ml) was added dropwise at 0° C. Mixture was stirred for overnight and then solvent was evaporated. Crude product was used in the next step without purification. M=12.1 g. Yield=100%.

¹H NMR: (CDCl₃, 400 MHz) δ=0.11 (s, 6H), 0.94 (s, 9H), 1.42 (s, 9H), 1.48 (s, 3H), 1.85 (br. s., 2H), 3.43-3.60 (m, 2H), 4.80 (d, J=1.2 Hz, 2H), 5.07 (br. s., 1H), 7.09 (t, J=1.2 Hz, 1H).

¹³C NMR: (CDCl₃, 100 MHz) δ=−5.2 (2C), 18.5, 26.0 (3C), 28.2, 28.5 (3C), 51.6, 57.5, 62.4, 79.6, 114.2, 156.6, 157.1, 179.1.

### Example 5.77

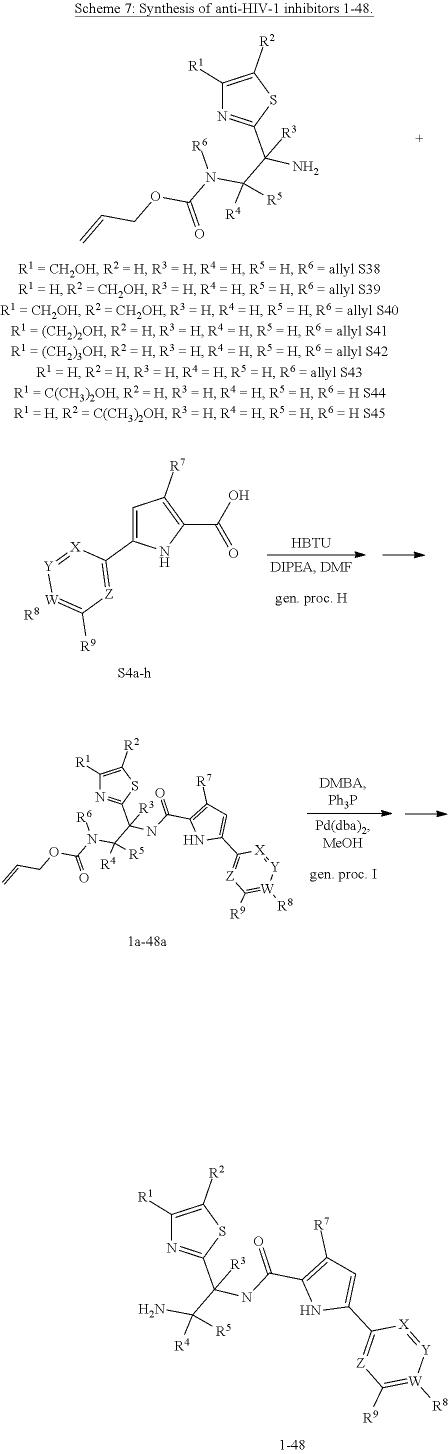

The amines S38-S40 were prepared using a method described in our earlier work. Similarly, amines S41-S43 were synthesized, starting from the appropriate thiazole derivative and following the same protocol as reported earlier [Eur J Med Chem. 2018 Jun. 25; 154:367-391. doi: 10.1016/j.ejmech.2018.04.062. Epub 2018 May 12], While amine S44 and S45 were synthesized from the appropriate starting thiazole following the procedure reported earlier [ChemMedChem. 2018 Nov. 6; 13(21):2332-2348. doi: 10.1002/cmdc.201800534. Epub 2018 Oct. 19.].

All the alloc protected intermediated were worked-up as per the gnarl procedure H [Eur J Med Chem. 2018 Jun. 25; 154:367-391. doi: 10.1016/j.ejmech.2018.04.062. Epub 2018 May 12] and the corresponding alloc products (1a-48a) were used directly in the next step without characterization. R groups and X, Y, Z and W atoms as per the Table 1.

### Example 5.78 General Procedure H: for Amide Coupling

DIPEA (1 equiv) was added to an appropriate acid (1 equiv) followed by DMF (10 mL per 1 g of acid) and then HBTU (1 equiv). The resulting solution was stirred for 10 min and added to a solution of appropriate amine (1 equiv) in DMF (10 mL per 1 g of amine) in several portions. The reaction mixture was stirred overnight; DMF was evaporated, and the residue was dissolved in DCM (50 mL per 1g of crude product) and successively washed with 5% aqueous NaOH and 10% tartaric acid solutions (25 mL per 1g of crude product). The organic layer was dried over Na₂SO₄, filtered, evaporated, and dry loaded on silica. Eluting with hexanes/EtOAc (1:1, then pure EtOAc) gave the target compounds. The products were used in the next step without analysis.

### Example 5.79 General Procedure I: for Deprotection

To a solution containing protected compound (1 equiv) and N,N-dimethyl barbituric acid (NDMBA, 3 equiv) in MeOH (0.1M solution), PPh₃ (10 mol %) was added under a nitrogen atmosphere followed by Pd(dba)₂ (5 mol %). The mixture was stirred for 1 day under reflux. After cooling, 50 mL of DCM was added, and the organic phase was shaken with 10% aqueous K₂CO₃ (50 mL) to remove the unreacted NDMBA. The organic layer was separated, and the aqueous layer was extracted with DCM/EtOH (˜4:1, (2-4)×50 mL). The combined organic layers were dried over Na₂SO₄, filtered, and concentrated. Purification by flash chromatography (eluent: DCM/MeOH (saturated with NH₃˜7M), 10:1) afforded amine as a slightly brown or yellowish solid.

### Example 5.80

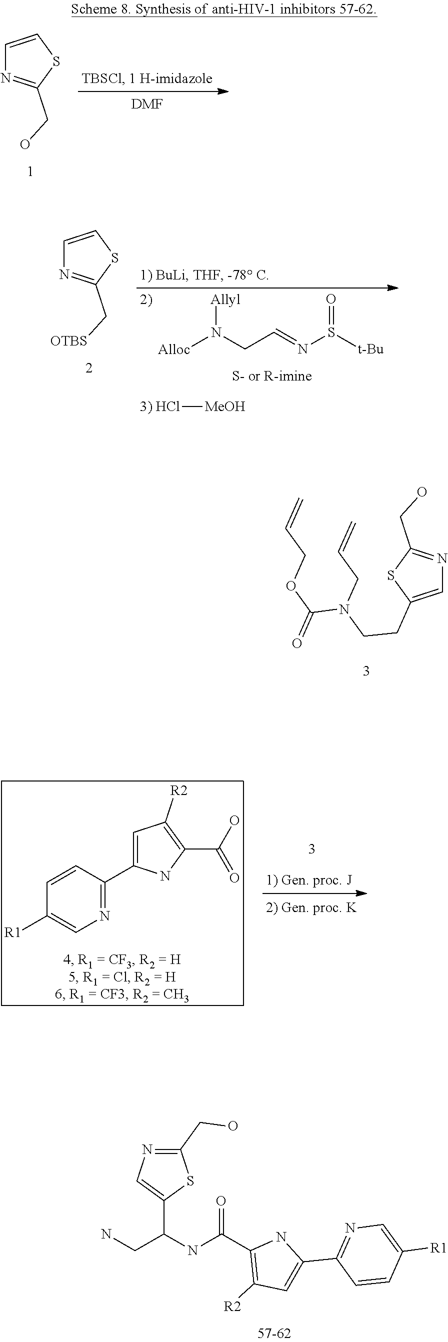

### Example 5.81 2-(((tert-Butyldimethylsilyl)oxy)methyl)thiazole (2)

To a solution of appropriate alcohol 1 (13.27 g, 117 mmol, 1 equiv.) DMF (120 mL), imidazole (10.4 g, 152 mmol, 1.3 equiv.) was added in one portion, followed by the portionwise addition of TBSCI (21.21 g, 141 mmol, 1.2 equiv.). The reaction mixture was stirred overnight at 50-60° C., cooled to room temperature, diluted with water (250 mL), and extracted with EtOAc (3×100 mL). The combined organic phases were washed with water (100 mL), brine (100 mL), and dried over anhydrous Na₂SO₄, filtered and evaporated to give an oil, which was purified by flash chromatography (eluent: hexane-EtOAc from 20:1 to 10:1, Rf=0.4 in hexane-EtOAc 10:1). M=24.82 g. Yield=92%.

¹H NMR (CDCl₃, 400 MHz): δ=0.13 (s, 6H), 0.96 (s, 9H), 4.99 (s, 2H), 7.27 (d, J=3.2 Hz, 1H), 7.72 (d, J=3.2 Hz, 1H).

¹³C NMR (CDCl₃, 100 MHz): δ=5.3 (2 C), 18.4, 25.9 (3 C), 63.4, 118.6, 142.6, 173.5.

### Example 5.82 Allyl allyl(2-amino-2-(2-(hydroxymethyl)thiazol-5-yl)ethyl)carbamate (3)

Experimental procedures for the synthesis of R- and S-enantiomers (fR and fS):

The thiazole 2 (8 g, 35 mmol, 1.05 equiv.) was dissolved in THF (35 mL) and cooled to −78° C. At this temperature, n-BuLi (2.5 M in hexane, 13.95 mL, 1.05 equiv.) was added dropwise under a nitrogen atmosphere. The reaction mixture was stirred for 20 min at −78° C., and appropriate R- or S-imine (9.51 g, 33 mmol, 1 equiv.) was added dropwise as a solution in THF (1M, 35 mL). The reaction mixture was slowly (˜1 h) warmed to 0° C. and poured into water (150 mL). The organic layer was separated, and the water layer was extracted with CH₂Cl₂ (3×100 mL). Combined organic phases were dried over Na₂SO₄, filtered, and evaporated to give a brown oil which was purified by flash chromatography (eluent: hexane-EtOAc, gradient from 5:1 to 0:1, Rf=0.6 in EtOAc). This product was used without analysis.

To a solution of protected thiazole from the previous step in MeOH (50 ml), 1M solution of HCl in MeOH (100 ml) was added in one portion. The mixture was stirred for 2-3 h, and the solvent was evaporated. The residue was dissolved in water (50 mL) and extracted with CH₂Cl₂ (2×50 mL). After that, solid K₂CO₃ was added carefully (CO₂ evolution!) to pH 10-12. Product was extracted from water with CH₂Cl₂ (4×50 mL), combined organic layers were dried over Na₂SO₄, filtered, and evaporated to give pure amine.

3-fR: M=3.95 g. Yield (over two steps)=39%.

3-fS: M=4.04 g. Yield (over two steps)=40%.

¹H NMR (DMSO-d₆, 400 MHz): 5=2.23 (br. s., 2H), 3.27-3.37 (m, 2H), 3.72 (dd, J=16.2, 5.3 Hz, 1H), 3.82-3.93 (m, 1H), 4.31-4.55 (m, 3H), 4.65 (s, 2H), 5.04-5.14 (m, 2H), 5.16 (dd, J=10.5, 1.5 Hz, 1H), 5.24 (dd, J=17.2, 1.4 Hz, 1H), 5.66-5.80 (m, 1H), 5.81-5.95 (m, 1H), 6.00 (br. s., 1H), 7.48 (s, 1H).

¹³C NMR (DMSO-d₆, 100 MHz): 5=(48.2, 48.6), 50.0, (54.0, 54.9), 61.0, 65.3, (116.1, 116.6), (116.7, 116.9), 133.4, (133.7, 134.0), 138.1, 143.3, (155.1, 155.5), 172.2.

### Example 5.83 General Procedure J

DIPEA (1 equiv.) was added to an appropriate acid (1 equiv.) followed by DMF (10 mL per 1 g of acid) and then HBTU (1 equiv.). The resulting solution was stirred for 5 min and added to a solution of appropriate amine (1 equiv.) in DMF (10 mL per 1 g of amine) in several portions. The reaction mixture was stirred overnight; DMF was evaporated, and the residue was dissolved in DCM (50 mL) and successively washed with 5% aqueous NaOH and 10% tartaric acid or citric acid aqueous solutions (50 mL). The organic layer was dried over Na₂SO₄, filtered, evaporated, and dry loaded on silica. Eluting with hexanes/EtOAc (1:1, then pure EtOAc) gave the target compounds. The products were used in the next step without analysis.

### Example 5.84 General Procedure K

To a solution containing protected compound (5 mmol) and N,N-dimethyl barbituric acid (NDMBA, 15 mmol, 3 equiv.) in MeOH (50 mL), PPh₃ (10 mol %) was added under a nitrogen atmosphere followed by Pd(dba)₂ (5 mol %). The mixture was stirred for 1 day under reflux. After cooling, 50 mL of DCM was added, and the organic phase was shaken with 10% aqueous K₂CO₃ (50 mL) to remove the unreacted NDMBA. The organic layer was separated, and the aqueous layer was extracted with DCM/EtOH (˜4:1). The combined organic layers were dried over Na₂SO₄, filtered, and concentrated. Purification by flash chromatography afforded amine as a slightly brown or yellowish solid.

### Example 5.85 N-(2-Amino-1-(2-(hydroxymethyl)thiazol-5-yl)ethyl)-5-(5-(trifluoromethyl)pyridin-2-yl)-1H-pyrrole-2-carboxamide

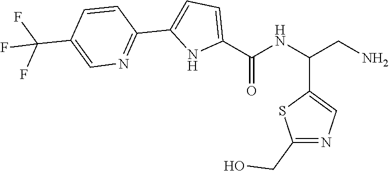 [SMILES: NCC(NC(=O)c1ccc(-c2ccc(C(F)(F)F)cn2)[nH]1)c1cnc(CO)s1]

Compounds 57 and 58 were obtained following the general procedure J and K from amine 3 and acid 4. Compounds were purified using column chromatography on silica gel (twice). Eluent 1: CHCl₃-MeOH (saturated with NH₃-7M), 10:1 and 5:1, Eluent 2: CH₂Cl₂-MeOH, 3:1 and 1:1. Acid 4 was prepared by following the methods reported previously (Curreli et al., J. Med. Chem. 2020, 63, 4, 1724-1749).

57; (R): M=670 mg. Yield=35% (over two steps). rt=1.093 min. Purity=100%. LC-MS: m/z [M+H]⁺=412 Da.

58; (S): M=771 mg. Yield=36% (over two steps). rt=1.101 min. Purity=100%. LC-MS: m/z [M+H]⁺=412 Da.

¹H NMR (DMSO-d₆, 400 MHz): 5=1.86 (br. s., 2H), 2.98 (d, J=6.7 Hz, 2H), 4.65 (s, 2H), 5.17-5.25 (m, 1H), 5.98 (br. s., 1H), 6.93 (d, J=3.9 Hz, 1H), 7.03 (d, J=3.9 Hz, 1H), 7.58 (d, J=0.7 Hz, 1H), 8.12 (d, J=8.5 Hz, 1H), 8.19 (dd, J=8.6, 2.2 Hz, 1H), 8.69 (d, J=8.0 Hz, 1H), 8.87-8.90 (m, 1H), 11.86 (br. s., 1H).

¹³C NMR (DMSO-d₆, 100 MHz): δ=46.9, 50.0, 61.0, 111.2, 114.0, 118.9, 122.3 (q, J=32.3 Hz), 124.0 (q, J=271.8 Hz), 129.6, 133.0, 134.4 (q, J=3.3 Hz), 139.0, 139.6, 146.0 (q, J=4.1 Hz), 153.2, 159.5, 172.5.

### Example 5.86 N-(2-Amino-1-(2-(hydroxymethyl)thiazol-5-yl)ethyl)-5-(5-chloropyridin-2-yl)-1H-pyrrole-2-carboxamide

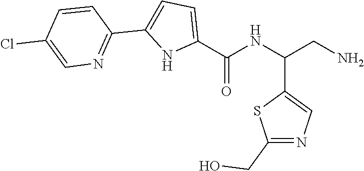 [SMILES: NCC(NC(=O)c1ccc(-c2ccc(Cl)cn2)[nH]1)c1cnc(CO)s1]

Compounds 59 and 60 were obtained following the general procedure J and K from amine 3 and acid 5. Compounds were purified using column chromatography on silica gel (twice). Eluent 1: CHCl₃-MeOH (saturated with NH₃˜7M), 10:1 and 5:1, Eluent 2: CH₂Cl₂-MeOH, 3:1 and 1:1. Acid 5 was prepared by following the methods reported previously¹.

59; (R): M=537 mg. Yield=41% (over two steps). rt=1.009 min. Purity=94%. LC-MS: m/z [M+H]⁺=378 Da.

60; (S): M=576 mg. Yield=44% (over two steps). rt=1.008 min. Purity=100%. LC-MS: m/z [M+H]⁺=378 Da.

¹H NMR (DMSO-d₆, 400 MHz): 5=1.67 (br. s., 2H), 2.96 (d, J=6.8 Hz, 2H), 4.64 (d, J=4.3 Hz, 2H), 5.15-5.23 (m, 1H), 5.97 (t, J=4.7 Hz, 1H), 6.88 (s, 2H), 7.57 (d, J=0.6 Hz, 1H), 7.91-7.98 (m, 2H), 8.58 (dd, J=1.9, 1.3 Hz, 1H), 8.62 (d, J=8.0 Hz, 1H), 11.86 (br. s., 1H).

¹³C NMR (DMSO-d₆, 100 MHz): 5=46.9, 49.9, 61.0, 109.6, 113.9, 120.3, 128.4, 128.6, 133.2, 136.8, 138.9, 139.7, 147.6, 148.4, 159.6, 172.4

### Example 5.87 N-(2-Amino-1-(2-(hydroxymethyl)thiazol-5-yl)ethyl)-3-methyl-5-(5-(trifluoromethyl)pyridin-2-yl)-1H-pyrrole-2-carboxamide

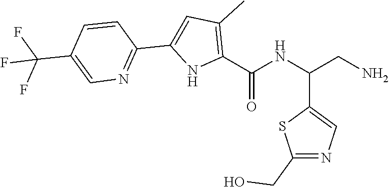 [SMILES: Cc1cc(-c2ccc(C(F)(F)F)cn2)[nH]c1C(=O)NC(CN)c1cnc(CO)s1]

Compounds 61 and 62 were obtained following the general procedure J and K from amine 3 and acid 6. Compounds were purified using column chromatography on silica gel (twice). Eluent 1: CHCl₃-MeOH (saturated with NH₃-7M), 10:1 and 5:1, Eluent 2: CH₂Cl₂-MeOH, 3:1 and 1:1. Acid 6 was prepared by following the methods reported previously¹.

61; (R): M=240 mg. Yield=22% (over two steps). rt=1.200 min. Purity=95%. LC-MS: m/z [M+H]⁺=426 Da.

62; (S): M=170 mg. Yield=16% (over two steps). rt=1.204 min. Purity=95%. LC-MS: m/z [M+H]⁺=426 Da.

¹H NMR (DMSO-d₆, 400 MHz): δ=2.30 (s, 3H), 3.01 (d, J=6.7 Hz, 2H), 4.64 (s, 2H), 5.23 (dd, J=13.9, 6.9 Hz, 1H), 5.99 (br. s, 1H), 6.87 (s, 1H), 7.59 (s, 1H), 8.02 (d, J=8.4 Hz, 1H), 8.17 (dd, J=8.6, 2.1 Hz, 1H), 8.73 (d, J=7.6 Hz, 1H), 8.85 (s, 1H), 11.95 (br. s, 1H).

¹³C NMR (DMSO-d₆, 100 MHz): δ=13.0, 46.2, 48.9, 60.9, 113.3, 118.9, 122.2 (q, J=32.1 Hz), 124.0 (q, J=271.8 Hz), 124.7, 127.1, 130.8, 134.4 (q, J=3.3 Hz), 139.1, 139.4, 145.8 (q, J=4.1 Hz), 153.1, 160.5, 172.6.

### Example 5.88

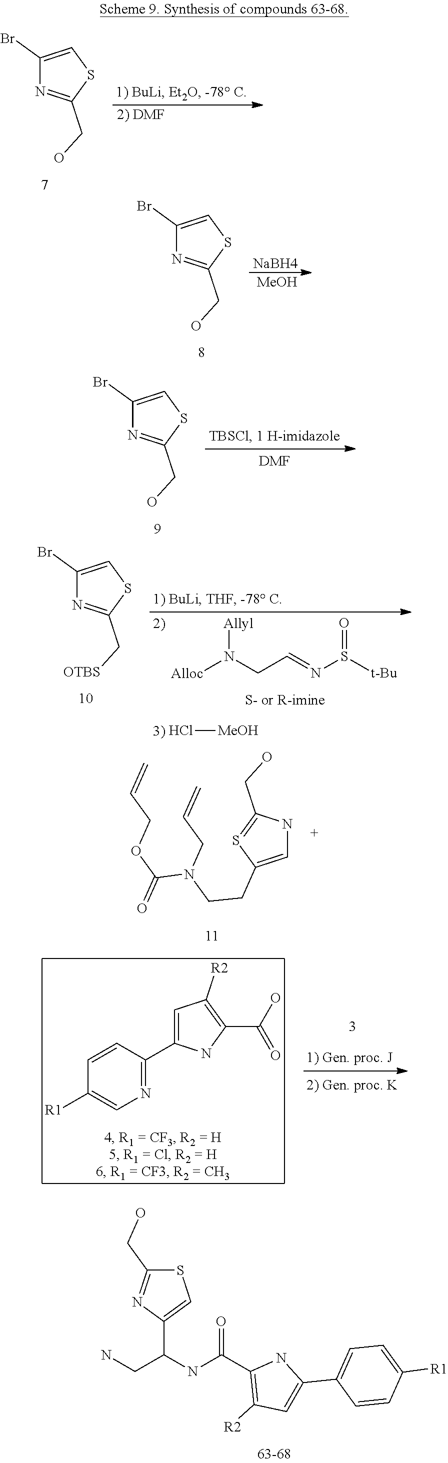

The acids (4-6 marked in the box in Scheme 9) were prepared by Scheme 3 (above).

### Example 5.89 4-Bromothiazole-2-carbaldehyde (8)

n-BuLi (51.5 mL, 1.05 equiv.) was added to a suspension of dibromide 7 (29.79 g, 1 equiv.) in dry diethyl ether (100 mL) at −80° C. under a nitrogen atmosphere. Dry DMF (10.43 mL, 1.1 equiv.) was added after stirring the suspension at −80° C. for 1 h. After an additional hour at −80° C. the reaction mixture was allowed to warm to room temperature. Dilute HCl (1 M, 2.1 equiv.) was added, and the mixture was stirred for 15 min. After separation, the aqueous layer was extracted with diethyl ether. The organic layer was washed with water, brine and dried over Na₂SO₄. Concentration by rotary evaporation gave crude product as a light brown solid (Rf=0.5 (SiO₂, hexane/ethyl acetate=3:1)). The crude product was purified by flash chromatography using Hexane-EA mixture as eluent (5:1 then 3:1). Yield 12.71 g (and 3.3 g of alcohol).

¹H NMR (CDCl₃, 400 MHz): 5=7.68 (d, J=1.2 Hz, 1H), 9.95 (d, J=1.2 Hz, 1H)

### Example 5. 90(4-Bromothiazol-2-yl)methanol (9)

NaBH₄ (3 g, 1.2 equiv.) was added to a stirring solution of aldehyde 8 (12.71 g, 1 equiv.) in MeOH (120 mL) in an ice bath. The reaction mixture was stirred at room temperature overnight. After that, the solvent was evaporated to a half volume, and saturated aqueous NH₄Cl (50 mL) was added to the residue and extracted with DCM (3×100 mL). The solvent was evaporated under vacuum to give the desired compound, which was used in the next step without further purification. Yield 11.6 g. Spectral data matched that previously reported for compound 9 (Fabrice et al., J. Med. Chem., 2007, 50, 14, 3256-3266).

¹H NMR (CDCl₃, 400 MHz): 5=4.02 (br. s., 1H), 4.94 (s, 2H), 7.20 (s, 1H)

¹³C NMR (CDCl₃, 100 MHz): 5=61.8, 117.0, 124.4, 173.0

### Example 5.91 4-Bromo-2-(((tert-butyldimethylsilyl)oxy)methyl)thiazole (10)

To a solution of appropriate alcohol (16.09 g, 83 mmol, 1 equiv.) DMF (100 mL), imidazole (7.9 g, 116 mmol, 1.4 equiv.) was added in one portion, followed by the portionwise addition of TBSCI (16.25 g, 108 mmol, 1.3 equiv.). The reaction mixture was stirred overnight at 50-60° C., cooled to room temperature, diluted with water (200 mL), and extracted with EtOAc (3×100 mL). The combined organic phases were washed with water (100 mL), brine (100 mL) and dried over anhydrous Na₂SO₄, filtered and evaporated to give an oil, which was purified by flash chromatography (eluent: hexane-EtOAc from 20:1 to 10:1, Rf=0.3 in hexane-EtOAc 20:1). M=22.1 g. Yield=86%.

¹H NMR (CDCl₃, 400 MHz): δ=0.13 (s, 6H), 0.95 (s, 9H), 4.94 (s, 2H), 7.18 (s, 1H).

¹³C NMR (CDCl₃, 100 MHz): δ=−5.4 (2 C), 18.4, 25.8 (3 C), 63.1, 116.6, 124.4, 174.7.

### Example 5.92 Allyl allyl(2-amino-2-(2-(hydroxymethyl)thiazol-4-yl)ethyl)carbamate (11)

The thiazole 10 (10 g, 32 mmol, 1.05 equiv.) was dissolved in Et₂O (30 mL) and cooled to −78° C. At this temperature, n-BuLi (2.5 M in hexane, 12.97 mL, 1.05 equiv.) was added dropwise under a nitrogen atmosphere. The reaction mixture was stirred for 20 min at −78° C., and appropriate R- or S-imine (8.85 g, 31 mmol, 1 equiv.) was added dropwise as a solution in Et₂O (1M, 30 mL). The reaction mixture was slowly (˜1 h) warmed to 0° C. and poured into water (150 mL). The organic layer was separated, and water was extracted with CH₂Cl₂ (3×100 mL). Combined organic phases were dried over Na₂SO₄, filtered, and evaporated to give a brown oil which was purified by column chromatography (eluent: hexane-EtOAc, gradient from 5:1 to 0:1, Rf=0.2 in hexane-EtOAc 1:1). This product was used without analysis.

To a solution of protected thiazole from the previous step in MeOH (50 ml), 1M solution of HCl in MeOH (100 ml) was added in one portion. The mixture was stirred for 2-3 h, and the solvent was evaporated. The residue was dissolved in water (50 mL) and extracted with CH₂Cl₂ (2×50 mL). After that, solid K₂CO₃ was added carefully (CO₂ evolution!) to pH 10-12. Product was extracted from water with CH₂Cl₂ (4×50 mL), combined organic layers were dried over Na₂SO₄, filtered, and evaporated to give pure amine.

11-fR: M=5.36 g. Yield (over two steps)=58%.

11-fS: M=4.97 g. Yield (over two steps)=54%.

¹H NMR (DMSO-d₆, 400 MHz): δ=1.90 (br. s., 2H), 3.27-3.49 (m, 2H), 3.68 (d, J=15.2 Hz, 1H), 3.85 (dd, J=16.4, 4.3 Hz, 1H), 4.07-4.16 (m, 1H), 4.42-4.56 (m, 2H), 4.69 (s, 2H), 5.01-5.12 (m, 2H), 5.16 (d, J=10.5 Hz, 1H), 5.27 (dd, J=15.5, 12.2 Hz, 1H), 5.66-5.80 (m, 1H), 5.83-5.95 (m, 1H), 6.07 (br. s., 1H), 7.32 (d, J=6.9 Hz, 1H).

¹³C NMR (DMSO-d₆, 100 MHz): δ=49.8, (50.9, 51.2), (52.6, 53.4), 60.9, 65.1, 113.6, (115.9, 116.4), (116.6, 116.7), 133.5, (133.9, 134.2), (155.2, 155.5), (159.4, 159.5), 173.6.

### Example 5.93 N-(2-Amino-1-(2-(hydroxymethyl)thiazol-4-yl)ethyl)-3-methyl-5-(5-(trifluoromethyl)pyridin-2-yl)-1H-pyrrole-2-carboxamide

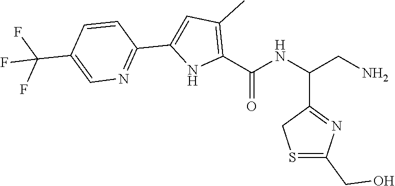

Compounds 63 and 64 were obtained following the general procedure J and K from amine 11 and acid 6. Compounds were purified using column chromatography on silica gel (twice). Eluent 1: CHCl₃-MeOH (saturated with NH₃˜7M), 10:1 and 5:1, Eluent 2: CH₂Cl₂-MeOH, 3:1 and 1:1. Acid 6 was prepared by following the methods reported previously¹.

63; (R): M=80 mg. Yield=25% (over two steps). rt=1.206 min. Purity=100%. LC-MS: m/z [M+H]⁺=426 Da.

64; (S): M=90 mg. Yield=27% (over two steps). rt=1.214 min. Purity=100%. LC-MS: m/z [M+H]⁺=426 Da.

¹H NMR (DMSO-d₆, 400 MHz): δ=2.30 (s, 3H), 2.96 (dd, J=13.0, 8.0 Hz, 1H), 3.04 (dd, J=13.1, 5.1 Hz, 1H), 4.70 (s, 2H), 5.10-5.18 (m, 1H), 6.06 (br. s, 1H), 6.87 (s, 1H), 7.39 (s, 1H), 8.03 (d, J=8.5 Hz, 1H), 8.17 (dd, J=8.5, 2.1 Hz, 1H), 8.67 (d, J=8.1 Hz, 1H), 8.86 (s, 1H), 12.00 (br. s, 1H).

¹³C NMR (DMSO-d₆, 100 MHz): δ=13.1, 44.9, 51.4, 60.9, 113.4, 114.9, 118.9, 122.1 (q, J=32.6 Hz), 124.0 (q, J=271.8 Hz), 125.0, 127.0, 130.7, 134.3 (q, J=3.3 Hz), 145.8 (q, J=4.0 Hz), 153.2, 155.5, 160.5, 174.1.

### Example 5.94 N-(2-Amino-1-(2-(hydroxymethyl)thiazol-4-yl)ethyl)-5-(5-(trifluoromethyl)pyridin-2-yl)-1H-pyrrole-2-carboxamide

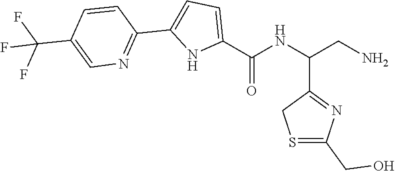

Compounds 65 and 66 were obtained following the general procedure J and K from amine 11 and acid 4. Compounds were purified using column chromatography on silica gel (twice). Eluent 1: CHCl₃-MeOH (saturated with NH₃˜7M), 10:1 and 5:1, Eluent 2: CH₂Cl₂-MeOH, 3:1 and 1:1. Acid 4 was prepared by following the methods reported previously¹.

65; (R): M=764 mg. Yield=52% (over two steps). rt=1.148 min. Purity=100%. LC-MS: m/z [M+H]+=412 Da.

66; (S): M=886 mg. Yield=50% (over two steps). rt=1.135 min. Purity=100%. LC-MS: m/z [M+H]⁺=412 Da.

¹H NMR (DMSO-d₆, 400 MHz): 5=0.61 (br. s., 2H), 2.92 (dd, J=13.0, 7.6 Hz, 1H), 2.98-3.05 (m, J=13.0, 5.4 Hz, 1H), 4.71 (s, 2H), 5.06-5.14 (m, 1H), 6.04 (br. s., 1H), 6.93 (d, J=3.8 Hz, 1H), 7.03 (d, J=3.9 Hz, 1H), 7.36 (s, 1H), 8.10 (d, J=8.5 Hz, 1H), 8.19 (dd, J=8.6, 2.0 Hz, 1H), 8.64 (d, J=8.2 Hz, 1H), 8.88 (s, 1H).

¹³C NMR (DMSO-d₆, 100 MHz): 5=45.8, 53.1, 60.9, 111.1, 114.1, 114.6, 118.8, 122.2 (q, J=32.3 Hz), 124.0 (q, J=271.8 Hz), 129.9, 132.8, 134.4 (q, J=3.1 Hz), 145.9 (q, J=4.1 Hz), 153.3, 156.2, 159.5, 173.8.

### Example 5.95 N-(2-Amino-1-(2-(hydroxymethyl)thiazol-4-yl)ethyl)-5-(5-chloropyridin-2-yl)-1H-pyrrole-2-carboxamide

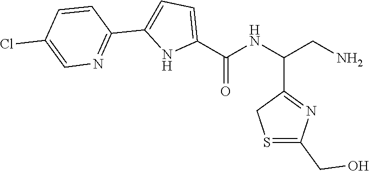

Compounds 67 and 68 were obtained following the general procedure J and K from amine 11 and acid 5. Compounds were purified using column chromatography on silica gel (twice). Eluent 1: CHCl₃-MeOH (saturated with NH₃˜7M), 10:1 and 5:1, Eluent 2: CH₂Cl₂-MeOH, 3:1 and 1:1. Acid 5 was prepared by following the methods reported previously¹.

67; (R): M=619 mg. Yield=47% (over two steps). rt=1.042 min. Purity=95%. LC-MS: m/z [M+H]⁺=378 Da.

68; (S): M=523 mg. Yield=40% (over two steps). rt=1.048 min. Purity=96%. LC-MS: m/z [M+H]+=378 Da.

¹H NMR (DMSO-d₆, 400 MHz): 5=1.55 (br. s., 2H), 2.91 (dd, J=13.0, 7.6 Hz, 1H), 2.96-3.04 (m, J=13.1, 5.4 Hz, 1H), 4.71 (s, 2H), 5.04-5.13 (m, 1H), 6.04 (br. s., 1H), 6.85-6.92 (m, 2H), 7.34 (s, 1H), 7.93 (d, J=1.5 Hz, 2H), 8.54-8.62 (m, 2H), 11.84 (br. s., 1H).

### Example 5.96

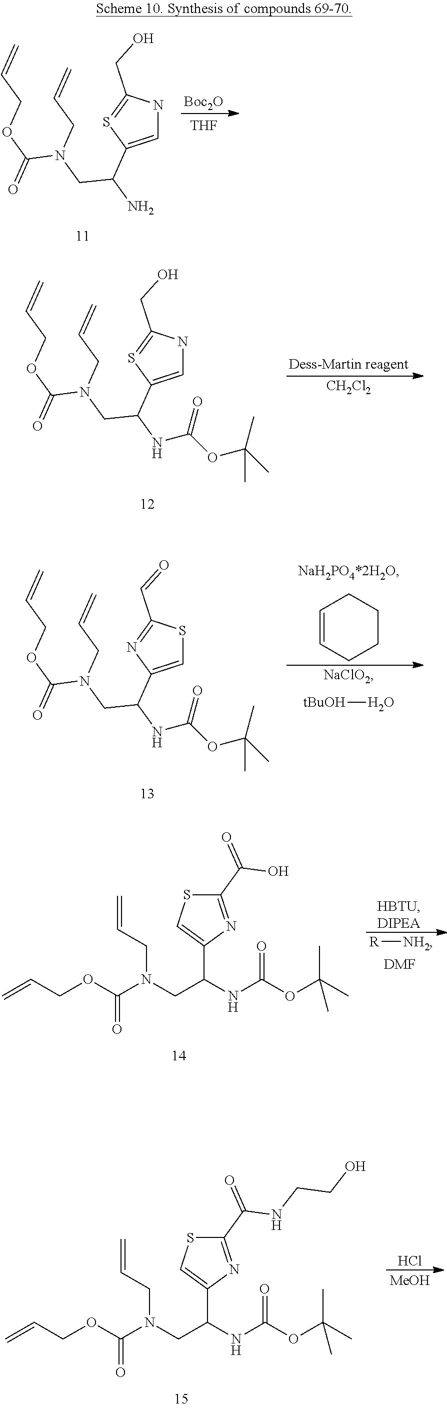

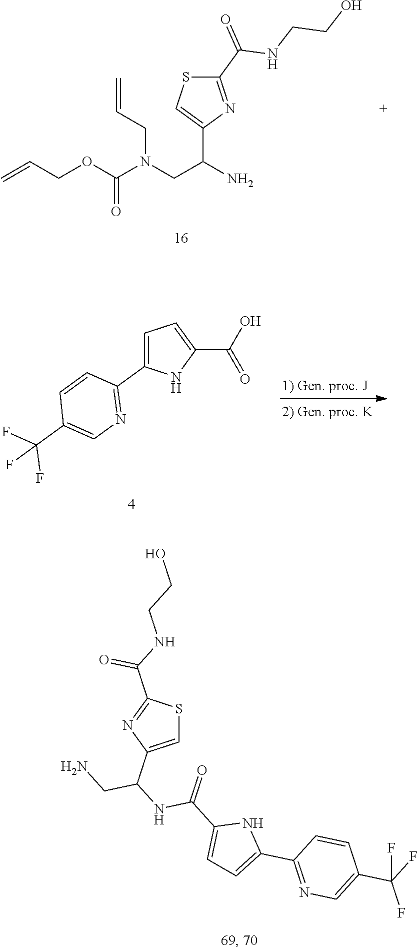 [SMILES: C=CCOC(=O)N(CC=C)CC(N)c1csc(C(=O)NCCO)n1 ; "+" ; O=C(O)c1ccc(-c2ccc(C(F)(F)F)cn2)[nH]1 ; NCC(NC(=O)c1ccc(-c2ccc(C(F)(F)F)cn2)[nH]1)c1csc(C(=O)NCCO)n1]

The acid (4) was prepared by Scheme 1 (above).

### Example 5.97 Allyl allyl(2-((tert-butoxycarbonyl)amino)-2-(2-(hydroxymethyl)thiazol-4-yl)ethyl)carbamate (12)

Amine 11 (1 equiv.) was dissolved in THF (0.1 M) and followed by Boc₂O (1.1 equiv.) was added in one portion. The reaction mixture was stirred overnight. The solvent was evaporated, and the crude product was purified by flash chromatography.

12-fR: M=1.02 g. Yield=71%.

13-fS: M=1.03 g. Yield=56%.

¹H NMR (CDCl₃, 400 MHz): 5=1.43 (s, 9H), 3.38-4.13 (m, 5H), 4.44-4.69 (m, 2H), 4.86 (d, J=3.8 Hz, 2H), 4.96-5.49 (m, 5H), 5.67-6.00 (m, 3H), 7.12 (s, 1H).

### Example 5.98 Allyl allyl(2-((tert-butoxycarbonyl)amino)-2-(2-formylthiazol-4-yl)ethyl)carbamate (13)

Starting alcohol 12 (1 equiv.) was dissolved in CH₂Cl₂ (0.5M), and Dess-Martin reagent (1.2 equiv.) was added in one portion. The mixture was stirred for 3-4 h (TLC-control) at room temperature. Saturated NaHCO₃ (50 mL) was added, and the organic layer was separated. Water was extracted with CH₂Cl₂ (3×50 mL); combined organic layers were dried over anhydrous Na₂SO₄ and evaporated. The crude product was purified by flash chromatography using Hexane-EtOAc as eluent (3:1, Rf=0.2 in that mixture).

13-fR: M=0.68 g. Yield=85%.

13-fS: M=0.83 g. Yield=81%.

¹H NMR (CDCl₃, 400 MHz): δ=1.43 (s, 9H), 3.46-4.00 (m, 4H), 4.39-4.70 (m, 2H), 5.03-5.44 (m, 5H), 5.65-6.00 (m, 3H), 7.51-7.63 (m, 1H), 9.94 (d, J=1.2 Hz, 1H).

### Example 5.99 4-(8-Allyl-2,2-dimethyl-4,9-dioxo-3,10-dioxa-5,8-diazatridec-12-en-6-yl)thiazole-2-carboxylic acid (14)

To a solution of aldehyde 13 (1 equiv.) in a mixture of water, t-BuOH, and THF (1:3:3, 0.2 M) cyclohexene (8 equiv.), potassium phosphate monobasic (3 equiv.), then NaClO₂ (3 equiv.) were added in that order. After four hours, the reaction is complete and diluted with water (100 mL). The aqueous solution was extracted with EtOAc (3×100 mL). Combined organic layers were dried over Na₂SO₄, and the solvent was evaporated. The crude product was used in the next step without further purification.

14-fR: M=0.60 g. Yield=85%.

13-fS: M=0.58 g. Yield=67%.

¹H NMR (DMSO-d₆, 400 MHz): δ=1.36 (s, 9H), 3.43-3.90 (m, 4H), 4.41-4.61 (m, 2H), 4.92-5.37 (m, 5H), 5.62-5.98 (m, 2H), 7.13-7.51 (m, 2H).

### Example 5.100 Allyl allyl(2-((tert-butoxycarbonyl)amino)-2-(2-((2-hydroxyethyl)carbamoyl)thiazol-4-yl)ethyl)carbamate (15)

DIPEA (1 equiv) was added to an acid 14 (1 equiv.) followed by DMF (10 mL per 1 g of acid) and then HBTU (1 equiv). The resulting solution was stirred for 5 min and added to a solution of appropriate amine (1 equiv.) in DMF (10 mL per 1 g of amine) in several portions. The reaction mixture was stirred overnight; DMF was evaporated, and the residue was dissolved in DCM (50 mL) and successively washed with 5% aqueous NaOH and 10% tartaric acid or citric acid aqueous solutions (50 mL). The organic layer was dried over Na₂SO₄, filtered, evaporated, and dry loaded on silica. The products were used in the next step without purification and analysis.

### Example 5.101 Allyl allyl(2-amino-2-(2-((2-hydroxyethyl)carbamoyl)thiazol-4-yl)ethyl)carbamate (16)

To a solution of protected thiazole 15 from the previous step in MeOH (10 ml), 1M solution of HCl in MeOH (100 ml) was added in one portion. The mixture was stirred for 2-3 h, and the solvent was evaporated. The residue was dissolved in water (50 mL) and extracted with CH₂Cl₂ (2×50 mL). After that, solid K₂CO₃ was added carefully (CO₂ evolution!) to pH 10-12. Product was extracted from water with CH₂Cl₂ (4×50 mL), combined organic layers were dried over Na₂SO₄, filtered, and evaporated to give pure amine.

16-fR: M=0.36 g. Yield (over two steps)=52%.

16-fS: M=0.30 g. Yield (over two steps)=60%.

¹H NMR (CDCl₃, 400 MHz): δ=1.63-2.03 (m, 3H), 3.45-4.00 (m, 8H), 4.30-4.69 (m, 3H), 5.02-5.40 (m, 4H), 5.66-5.98 (m, 2H), 7.35-7.79 (m, 1H).

¹³C NMR (DMSO-d₆, 100 MHz): 5=45.8, 53.0, 60.9, 109.6, 114.0, 114.6, 120.3, 128.4, 129.0, 133.0, 136.8, 147.5, 148.5, 156.2, 159.6, 173.

### Example 5.102 4-(2-Amino-1-(5-(5-(trifluoromethyl)pyridin-2-yl)-1H-pyrrole-2-carboxamido)ethyl)-N-(2-hydroxyethyl)thiazole-2-carboxamide

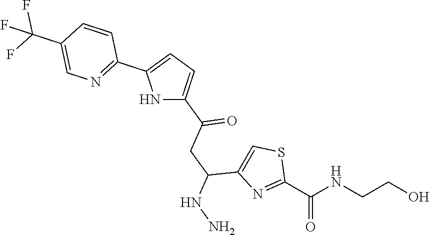 [SMILES: NNC(CC(=O)c1ccc(-c2ccc(C(F)(F)F)cn2)[nH]1)c1csc(C(=O)NCCO)n1]

Compounds 69 and 70 were obtained following the general procedure J and K from amine 16 and acid 4. Compounds were purified using column chromatography on silica gel (twice). Eluent 1: CHCl₃-MeOH (saturated with NH₃˜7M), 10:1 and 5:1, Eluent 2: CH₂Cl₂-MeOH, 3:1 and 1:1. Acid 4 was prepared by following the methods reported previously¹.

69; (R): M=82 mg. Yield=17% (over two steps). rt=1.200 min. Purity=100%. LC-MS: m/z [M+H]+=469 Da.

70; (S): M=72 mg. Yield=18% (over two steps). rt=1.137 min. Purity=100%. LC-MS: m/z [M+H]+=469 Da.

¹H NMR (DMSO-d₆, 400 MHz): 5=1.50 (s, 2H), 2.97 (dd, J=13.1, 7.3 Hz, 1H), 3.09 (dd, J=13.2, 5.3 Hz, 1H), 3.35-3.39 (m, 2H), 3.48-3.54 (m, 2H), 4.80 (t, J=4.9 Hz, 1H), 5.13-5.20 (m, 1H), 6.94 (d, J=3.9 Hz, 1H), 7.04 (d, J=3.9 Hz, 1H), 7.74 (s, 1H), 8.10 (d, J=8.5 Hz, 1H), 8.15-8.22 (m, J=8.7, 2.3 Hz, 1H), 8.53 (t, J=6.1 Hz, 1H), 8.65 (d, J=7.9 Hz, 1H), 8.88 (s, 1H), 11.97 (br. s., 1H).

¹³C NMR (DMSO-d₆, 100 MHz): 5=41.8, 45.7, 53.2, 59.5, 111.2, 114.2, 118.9, 120.8, 122.3 (q, J=32.3 Hz), 124.0 (q, J=271.8 Hz), 129.8, 132.9, 134.5 (q, J=3.3 Hz), 146.0 (q, J=4.4 Hz), 153.3, 157.9, 159.2, 159.6, 163.5.

### Example 6. Compound Synthesis

### Example 6.1 N-(2-Amino-1-(5-(hydroxymethyl)thiazol-2-yl)ethyl)-5-(5-chloropyridin-2-yl)-1H-pyrrole-2-carboxamide (1 & 2)

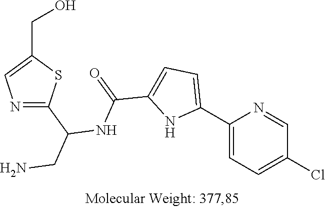 [SMILES: NCC(NC(=O)c1ccc(-c2ccc(Cl)cn2)[nH]1)c1ncc(CO)s1]

Compounds 1 and 2 were obtained following the general procedure H and I from amine S39 and acid S4b. Compounds were purified using column chromatography on silica gel. Eluent CHCl₃-MeOH saturated with NH₃ (10:1 and 5:1).

1; (fR): M=507 mg. Yield=33% (over two steps). rt=1.035 min. Purity=100%. LC-MS: m/z [M+H]⁺=378 Da.

2; (fS): M=335 mg. Yield=22% (over two steps). rt=1.022 min. Purity=100%. LC-MS: m/z [M+H]⁺=378 Da.

¹H NMR (DMSO-d₆, 400 MHz): δ=1.68 (br. s., 2H), 2.97 (dd, J=13.2, 7.9 Hz, 1H), 3.11 (dd, J=13.3, 5.3 Hz, 1H), 4.59 (d, J=4.2 Hz, 2H), 5.12-5.20 (m, 1H), 5.46 (t, J=5.3 Hz, 1H), 6.88-6.94 (m, 2H), 7.54 (s, 1H), 7.89-8.01 (m, 2H), 8.52-8.64 (m, 1H), 8.80 (d, J=7.5 Hz, 1H), 11.89 (br. s., 1H).

¹³C NMR (DMSO-d₆, 100 MHz): δ=45.8, 54.7, 55.8, 109.7, 114.3, 120.4, 128.4, 128.5, 133.4, 136.9, 139.1, 140.1, 147.6, 148.4, 160.0, 171.8.

HRMS (ESI) calcd for C₁₆H₁₇ClN₅O₂S [M+H]⁺ 378.0786, found 378.0787.

### Example 6.2 N-(2-Amino-1-(5-(hydroxymethyl)thiazol-2-yl)ethyl)-5-(5-chloropyridin-2-yl)-3-methyl-1H-pyrrole-2-carboxamide (3 & 4)

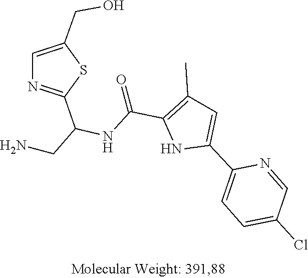 [SMILES: Cc1cc(-c2ccc(Cl)cn2)[nH]c1C(=O)NC(CN)c1ncc(CO)s1]

Compounds 3 and 4 were obtained following the general procedure H and I from amine S39 and acid S16. Compounds were purified using column chromatography on silica gel. Eluent CHCl₃-MeOH saturated with NH₃ (10:1 and 5:1).

3; (fR): M=412 mg. Yield=26% (over two steps). rt=1.112 min. Purity=100%. LC-MS: m/z [M+H]⁺=392 Da.

4; (fS): M=284 mg. Yield=18% (over two steps). rt=1.140 min. Purity=100%. LC-MS: m/z [M+H]⁺=392 Da.

¹H NMR (DMSO-d₆, 400 MHz): δ=2.31 (s, 3H), 3.00 (dd, J=13.2, 8.1 Hz, 1H), 3.13 (dd, J=13.3, 5.1 Hz, 1H), 4.60 (s, 2H), 5.13-5.23 (m, 1H), 5.49 (br. s., 1H), 6.76 (s, 1H), 7.55 (s, 1H), 7.82-7.87 (m, 1H), 7.92-7.96 (m, 1H), 8.59 (d, J=2.3 Hz, 1H), 8.76 (d, J=7.5 Hz, 1H), 11.80 (br. s., 1H).

¹³C NMR (DMSO-d₆, 100 MHz): δ=13.1, 45.7, 54.2, 55.8, 111.9, 120.4, 123.5, 127.2, 128.4, 131.2, 136.9, 139.1, 140.1, 147.4, 148.3, 160.8, 172.0.

HRMS (ESI) calcd for C₁₇H₁₉ClN₅O₂S [M+H]⁺ 392.0942, found 392.0942.

### Example 6.3 N-(2-Amino-1-(5-(hydroxymethyl)thiazol-2-yl)ethyl)-5-(5-(trifluoromethyl)pyridin-2-yl)-1H-pyrrole-2-carboxamide (5 & 6)

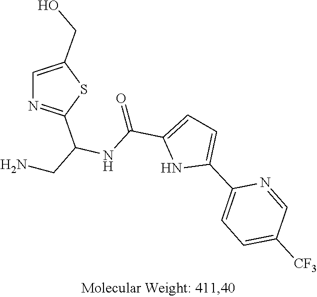 [SMILES: NCC(NC(=O)c1ccc(-c2ccc(C(F)(F)F)cn2)[nH]1)c1ncc(CO)s1]

Compounds 5 and 6 were obtained following the general procedure H and I from amine S39 and acid S4d. Compounds were purified using column chromatography on silica gel. Eluent CHCl₃-MeOH saturated with NH₃ (10:1 and 5:1).

5; (fR): M=262 mg. Yield=38% (over two steps). rt=1.260 min. Purity=100%. LC-MS: m/z [M+H]⁺=412 Da.

6; (fS): M=162 mg. Yield=24% (over two steps). rt=1.265 min. Purity=100%. LC-MS: m/z [M+H]⁺=412 Da.

¹H NMR (DMSO-d₆, 400 MHz): δ=3.02 (dd, J=13.2, 7.8 Hz, 1H), 3.15 (dd, J=13.4, 5.4 Hz, 1H), 4.62 (s, 2H), 5.15-5.26 (m, 1H), 5.51 (br. s., 1H), 6.99 (d, J=3.9 Hz, 1H), 7.05 (d, J=3.9 Hz, 1H), 7.56 (s, 1H), 8.12 (d, J=8.4 Hz, 1H), 8.19 (dd, J=8.6, 2.1 Hz, 1H), 8.81-8.97 (m, 2H).

¹³C NMR (DMSO-d₆, 100 MHz): δ=45.8, 54.7, 55.8, 111.2, 114.3, 118.9, 122.4 (q, J=32.1 Hz), 124.0 (q, J=271.5 Hz), 129.4, 133.1, 134.4 (q, J=3.2 Hz), 139.1, 140.1, 146.0 (q, J=4.0 Hz), 153.2, 159.9, 171.7.

HRMS (ESI) calcd for C₁₇H₁₇F₃N₅O₂S [M+H]⁺ 412.1050, found 412.1048.

### Example 6.4 N-(2-amino-1-(5-(hydroxymethyl)thiazol-2-yl)ethyl)-3-methyl-5-(5-(trifluoromethyl)pyridin-2-yl)-1H-pyrrole-2-carboxamide (7 & 8)

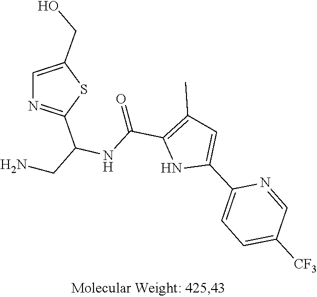 [SMILES: Cc1cc(-c2ccc(C(F)(F)F)cn2)[nH]c1C(=O)NC(CN)c1ncc(CO)s1]

Compounds 7 and 8 were obtained following the general procedure H and I from amine S39 and the sodium salt S15. Compounds were purified using column chromatography on silica gel. Eluent CHCl₃-MeOH saturated with NH₃ (10:1 and 5:1).

7; (fR): M=358 mg. Yield=40% (over two steps). rt=1.202 min. Purity=100%. LC-MS: m/z [M+H]⁺=426 Da.

8); (fS): M=253 mg. Yield=28% (over two steps). rt=1.208 min. Purity=100%. LC-MS: m/z [M+H]⁺=426 Da.

¹H NMR (DMSO-d₆, 400 MHz): δ=1.93 (br. s., 2H), 2.33 (s, 3H), 3.00 (dd, J=13.2, 8.0 Hz, 1H), 3.12 (dd, J=13.3, 5.1 Hz, 1H), 4.61 (s, 2H), 5.13-5.22 (m, 1H), 5.50 (br. s., 1H), 6.91 (s, 1H), 7.56 (s, 1H), 8.00 (d, J=8.4 Hz, 1H), 8.19 (dd, J=8.5, 2.1 Hz, 1H), 8.80 (d, J=7.2 Hz, 1H), 8.89 (s, 1H), 12.00 (br. s., 1H).

¹³C NMR (DMSO-d₆, 100 MHz): δ=13.1, 45.7, 54.2, 55.8, 113.4, 119.0, 122.3 (q, J=32.4 Hz), 124.0 (q, J=271.8 Hz), 124.6, 127.3, 130.9, 134.4 (q, J=3.3 Hz), 139.1, 140.1, 145.8 (q, J=4.1 Hz), 153.1 (q, J=1.1 Hz), 160.8, 171.8.

HRMS (ESI) calcd for C₁₈H₁₉F₃N₅O₂S [M+H]⁺ 426.1206, found 426.1216.

### Example 6.5 N-(2-Amino-1-(5-(hydroxymethyl)thiazol-2-yl)-2-methylpropyl)-5-(5-(trifluoromethyl)pyridin-2-yl)-1H-pyrrole-2-carboxamide (9 & 10)

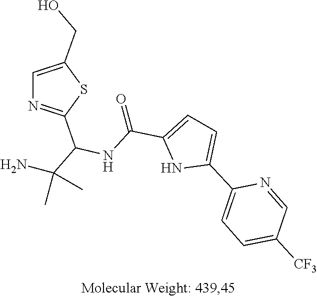 [SMILES: CC(C)(N)C(NC(=O)c1ccc(-c2ccc(C(F)(F)F)cn2)[nH]1)c1ncc(CO)s1]

Compounds 9 and 10 were obtained following the general procedure H and I from amine S45 and acid S4d. Compounds were purified using column chromatography on silica gel. Eluent CHCl₃-MeOH saturated with NH₃ (20:1 and 10:1).

9; (fR): M=434 mg. Yield=25% (over two steps). rt=1.204 min. Purity=100%. LC-MS: m/z [M+H]⁺=440 Da.

10; (fS): M=252 mg. Yield=15% (over two steps). rt=1.218 min. Purity=96%. LC-MS: m/z [M+H]⁺=440 Da.

¹H NMR (DMSO-d₆, 400 MHz): δ=1.10 (s, 3H), 1.14 (s, 3H), 4.63 (s, 2H), 5.29 (d, J=4.9 Hz, 1H), 5.51 (br. s., 1H), 6.96 (d, J=3.9 Hz, 1H), 7.05 (d, J=3.8 Hz, 1H), 7.58 (s, 1H), 8.11 (d, J=8.4 Hz, 1H), 8.20 (dd, J=8.4, 2.2 Hz, 1H), 8.64 (d, J=7.2 Hz, 1H), 8.88-8.92 (m, 1H).

¹³C NMR (DMSO-d₆, 100 MHz): δ=27.6, 28.3, 52.8, 55.8, 59.7, 111.2, 115.1, 119.1, 122.4 (q, J=32.2 Hz), 124.0 (q, J=272.1 Hz), 129.3, 133.2, 134.5 (q, J=3.2 Hz), 138.7, 140.3, 145.9 (q, J=4.1 Hz), 153.3, 159.4, 169.9.

HRMS (ESI) calcd for C₁₉H₂₁F₃N₅O₂S [M+H]⁺ 440.1363, found 440.1359.

### Example 6.6 N-(2-Amino-1-(5-(hydroxymethyl)thiazol-2-yl)-2-methylpropyl)-3-methyl-5-(5-(trifluoro-methyl)pyridin-2-yl)-1H-pyrrole-2-carboxamide (11 & 12)

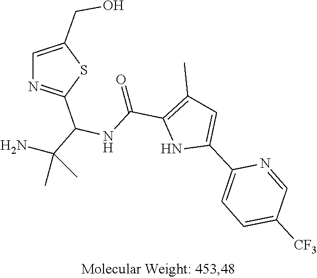 [SMILES: Cc1cc(-c2ccc(C(F)(F)F)cn2)[nH]c1C(=O)NC(c1ncc(CO)s1)C(C)(C)N]

Compounds 11 and 12 were obtained following the general procedure H and I from amine S45 and the sodium salt S15. Compounds were purified using column chromatography on silica gel. Eluent CHCl₃-MeOH saturated with NH₃ (20:1 and 10:1).

11; (fR): M=609 mg. Yield=32% (over two steps). rt=1.268 min. Purity=100%. LC-MS: m/z [M+H]⁺=454 Da.

12; (fS): M=674 mg. Yield=33% (over two steps). rt=1.306 min. Purity=100%. LC-MS: m/z [M+H]⁺=454 Da.

¹H NMR (DMSO-d₆, 400 MHz): δ=1.09 (s, 3H), 1.14 (s, 3H), 2.32 (s, 3H), 4.63 (d, J=3.5 Hz, 2H), 5.28 (s, 1H), 5.45-5.56 (m, 1H), 6.91 (s, 1H), 7.58 (s, 1H), 8.00 (d, J=8.4 Hz, 1H), 8.19 (dd, J=8.6, 2.2 Hz, 1H), 8.50 (br. s., 1H), 8.86-8.92 (m, 1H), 12.24 (br. s., 1H).

¹³C NMR (DMSO-d₆, 100 MHz): δ=13.4, 27.6, 28.1, 52.9, 55.8, 59.2, 113.6, 119.2, 122.3 (q, J=32.3 Hz), 124.0 (q, J=271.8 Hz) 124.5, 127.5, 131.0, 134.4 (q, J=3.1 Hz), 138.7, 140.3, 145.8 (q, J=4.1 Hz), 153.2, 160.4, 170.1.

HRMS (ESI) calcd for C₂₀H₂₃F₃N₅O₂S [M+H]⁺ 454.1519, found 454.1519.

### Example 6.7 N-(2-Amino-1-(5-(1,2-dihydroxyethyl)thiazol-2-yl)ethyl)-5-(5-(trifluoromethyl)pyridin-2-yl)-1H-pyrrole-2-carboxamide (13 & 14)

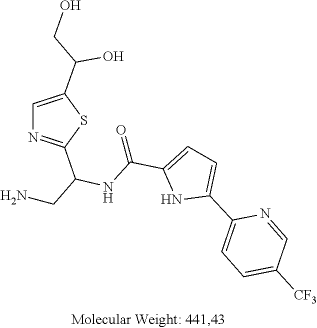 [SMILES: NCC(NC(=O)c1ccc(-c2ccc(C(F)(F)F)cn2)[nH]1)c1ncc(C(O)CO)s1]

Compounds 13 and were 14 obtained from amine S23 and acid S4d, following in sequence the general procedure H, I and the TBDPS cleavage as described below. Compounds were purified using column chromatography on silica gel. Eluent CHCl₃-MeOH saturated with NH₃ (10:1 and 5:1).

TBDPS cleavage: To a solution of TBDPS-protected compound (1 equiv) in THF (0.1 M), solution of TBAF trihydrate (1.1 equiv) in THF (0.1M) was added in a one portion. The mixture was stirred for 1-2 h at room temperature (TLC-control) and concentrated. Purification by flash chromatography using CHCl₃-MeOH saturated with NH₃ mixture (5:1 and 3:1) as eluent.

Note that compounds 13 & 14 were obtained as diastereisomeric mixture of 2 single compounds having the absolute configuration of the chiral carbon a as fR for 13 and fS for 14.

13; (fR): M=221 mg. Yield=27% (over three steps). rt=1.107 min. Purity=97%. LC-MS: m/z [M+H]⁺=442 Da.

14; (fS): M=485 mg. Yield=41% (over three steps). rt=1.160 min. Purity=100%. LC-MS: m/z [M+H]⁺=442 Da.

¹H NMR (DMSO-d₆, 400 MHz): δ=2.99 (dd, 1H), 3.13 (dd, J=13.2, 5.2 Hz, 1H), 3.42 (ddd, J=10.7, 5.7, 2.4 Hz, 1H), 3.50 (dd, J=10.8, 6.1 Hz, 1H), 4.75 (t, J=5.8 Hz, 1H), 4.96 (br. s., 1H), 5.14-5.24 (m, 1H), 5.71 (br. s., 1H), 6.98 (d, J=3.9 Hz, 1H), 7.05 (d, J=3.9 Hz, 1H), 7.57 (s, 1H), 8.13 (d, J=8.4 Hz, 1H), 8.20 (dd, J=8.6, 2.1 Hz, 1H), 8.83-8.93 (m, 2H).

¹³C NMR (DMSO-d₆, 100 MHz): δ=(45.8, 45.8), 54.6, 66.7, (68.1, 68.1), 111.3, 114.3, 119.0, 122.4 (q, J=32.3 Hz), 124.0 (q, J=271.8 Hz), 129.4, 133.2, 134.5 (q, J=3.3 Hz), (138.6, 138.6), (141.4, 141.4), 146.0 (q, J=4.1 Hz), (153.2, 153.2), 159.9, (171.0, 171.1).

HRMS (ESI) calcd for C₁₈H₁₉F₃N₅O₃S [M+H]⁺ 442.1155, found 442.1155.

### Example 6.8 N-(2-Amino-1-(4-(hydroxymethyl)thiazol-2-yl)ethyl)-5-(5-chloropyridin-2-yl)-1H-pyrrole-2-carboxamide (15 & 16)

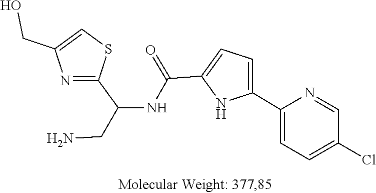 [SMILES: NCC(NC(=O)c1ccc(-c2ccc(Cl)cn2)[nH]1)c1nc(CO)cs1]

Compounds 15 and 16 were obtained following the general procedure H and I from amine S38 and acid S4b. Compounds were purified using column chromatography on silica gel. Eluent CHCl₃-MeOH saturated with NH₃ (10:1 and 5:1).

15; (fR): M=518 mg. Yield=34% (over two steps). rt=1.074 min. Purity=100%. LC-MS: m/z [M+H]⁺=378 Da.

16; (fS): M=381 mg. Yield=25% (over two steps). rt=1.079 min. Purity=100%.

LC-MS: m/z [M+H]⁺=378 Da.

¹H NMR (DMSO-d₆, 400 MHz): δ=1.73 (br. s., 2H), 2.99 (dd, J=13.2, 7.8 Hz, 1H), 3.12 (dd, J=13.2, 5.3 Hz, 1H), 4.54 (s, 2H), 5.17-5.24 (m, 1H), 5.30 (br. s., 1H), 6.85-6.97 (m, 2H), 7.29 (s, 1H), 7.89-8.00 (m, 2H), 8.59 (s, 1H), 8.83 (d, J=7.7 Hz, 1H), 11.91 (br. s., 1H).

¹³C NMR (DMSO-d₆, 100 MHz): δ=45.8, 54.3, 59.8, 109.7, 114.2, 114.3, 120.4, 128.4, 128.6, 133.5, 136.9, 147.6, 148.4, 157.7, 160.0, 172.0.

HRMS (ESI) calcd for C₁₆H₁₇ClN₅O₂S [M+H]⁺ 378.0786, found 378.0786.

### Example 6.9 N-(2-Amino-1-(4-(hydroxymethyl)thiazol-2-yl)ethyl)-5-(5-fluoropyridin-2-yl)-1H-pyrrole-2-carboxamide (17 & 18)

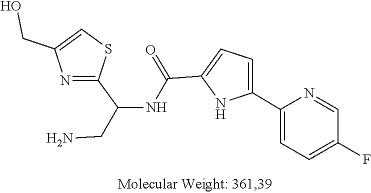 [SMILES: NCC(NC(=O)c1ccc(-c2ccc(F)cn2)[nH]1)c1nc(CO)cs1]

Compounds 17 and 18 were obtained following the general procedure H and I from amine S38 and acid S4c. Compounds were purified using column chromatography on silica gel. Eluent CHCl₃-MeOH saturated with NH₃ (10:1 and 5:1).

17; (fR): M=420 mg. Yield=31% (over two steps). rt=0.987 min. Purity=100%. LC-MS: m/z [M+H]⁺=362 Da.

18; (fS): M=325 mg. Yield=24% (over two steps). rt=1.001 min. Purity=96%. LC-MS: m/z [M+H]⁺=362 Da.

¹H NMR (DMSO-d₆, 400 MHz): δ=1.78 (br. s., 2H), 2.99 (dd, J=13.2, 7.8 Hz, 1H), 3.12 (dd, J=13.2, 5.3 Hz, 1H), 4.54 (d, J=3.0 Hz, 2H), 5.17-5.24 (m, 1H), 5.30 (br. s., 1H), 6.84 (d, J=3.9 Hz, 1H), 6.92 (d, J=3.8 Hz, 1H), 7.28 (s, 1H), 7.77 (td, J=8.8, 2.9 Hz, 1H), 7.99 (dd, J=8.9, 4.4 Hz, 1H), 8.55 (d, J=2.9 Hz, 1H), 8.80 (d, J=7.8 Hz, 1H), 11.82 (br. s., 1H).

¹³C NMR (DMSO-d₆, 100 MHz): δ=45.9, 54.4, 59.8, 109.0, 114.1, 114.2, 120.6 (d, J=4.6 Hz), 124.3 (d, J=19.0 Hz), 128.0, 133.7, 137.0 (d, J=24.0 Hz), 146.6 (d, J=3.7 Hz), 157.7, 157.9 (d, J=252.5 Hz), 160.1, 172.1.

HRMS (ESI) calcd for C₁₆H₁₇FN₅O₂S [M+H]⁺ 362.1081, found 362.1080.

### Example 6.10 N-(2-Amino-1-(4-(hydroxymethyl)thiazol-2-yl)ethyl)-5-(5-methylpyridin-2-yl)-1H-pyrrole-2-carboxamide (19 and 20)

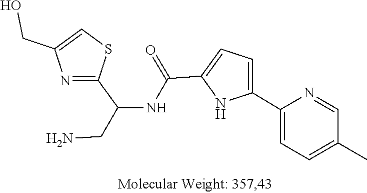 [SMILES: Cc1ccc(-c2ccc(C(=O)NC(CN)c3nc(CO)cs3)[nH]2)nc1]

Compounds 19 and 20 were obtained following the general procedure H and I from amine S38 and acid S4a. Compounds were purified using column chromatography on silica gel. Eluent CHCl₃-MeOH saturated with NH₃ (10:1 and 5:1).

19; (fR): M=357 mg. Yield=35% (over two steps). rt=0.829 min. Purity=100%. LC-MS: m/z [M+H]⁺=358 Da.

20; (fS): M=254 mg. Yield=25% (over two steps). rt=0.773 min. Purity=100%. LC-MS: m/z [M+H]⁺=358 Da.

¹H NMR (DMSO-d₆, 400 MHz): δ=1.71 (br. s., 2H), 2.30 (s, 3H), 2.99 (dd, J=13.2, 7.8 Hz, 1H), 3.12 (dd, J=13.2, 5.3 Hz, 1H), 4.54 (s, 2H), 5.16-5.25 (m, 1H), 5.32 (br. s., 1H), 6.81 (d, J=3.8 Hz, 1H), 6.90 (d, J=3.8 Hz, 1H), 7.29 (s, 1H), 7.63 (dd, J=8.2, 2.0 Hz, 1H), 7.79 (d, J=8.1 Hz, 1H), 8.41 (d, J=1.7 Hz, 1H), 8.82 (d, J=7.8 Hz, 1H), 11.78 (br. s., 1H).

¹³C NMR (DMSO-d₆, 100 MHz): δ=17.8, 46.0, 54.5, 59.8, 108.3, 114.0, 114.3, 118.7, 127.4, 131.0, 134.6, 137.4, 147.2, 149.2, 157.6, 160.0, 172.2.

HRMS (ESI) calcd for C₁₇H₂₀N₅O₂S [M+H]⁺ 358.1332, found 358.1337.

### Example 6.11 N-(2-Amino-1-(4-(hydroxymethyl)thiazol-2-yl)ethyl)-5-(5-(trifluoromethyl)pyridin-2-yl)-1H-pyrrole-2-carboxamide (21 & 22)

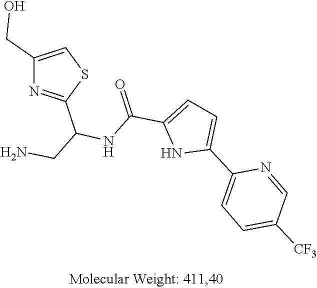 [SMILES: NCC(NC(=O)c1ccc(-c2ccc(C(F)(F)F)cn2)[nH]1)c1nc(CO)cs1]

Compounds 21 and 22 were obtained following the general procedure H and I from amine S38 and acid S4d. Compounds were purified using column chromatography on silica gel. Eluent CHCl₃-MeOH saturated with NH₃ (10:1 and 5:1).

21; (fR): M=410 mg. Yield=41% (over two steps). rt=1.269 min. Purity=100%. LC-MS: m/z [M+H]⁺=412 Da.

22; (fS): M=312 mg. Yield=32% (over two steps). rt=1.266 min. Purity=100%. LC-MS: m/z [M+H]⁺=412 Da.

¹H NMR (DMSO-d₆, 400 MHz): δ=3.00 (dd, J=13.2, 7.8 Hz, 1H), 3.14 (dd, J=13.2, 5.3 Hz, 1H), 4.55 (s, 2H), 5.18-5.26 (m, 1H), 5.32 (br. s., 1H), 6.98 (d, J=3.8 Hz, 1H), 7.05 (d, J=3.9 Hz, 1H), 7.29 (s, 1H), 8.13 (d, J=8.4 Hz, 1H), 8.20 (dd, J=8.6, 1.7 Hz, 1H), 8.84-8.96 (m, 2H).

¹³C NMR (DMSO-d₆, 100 MHz): δ=45.9, 54.4, 59.8, 111.2, 114.1, 114.3, 118.9, 122.3 (q, J=32.2 Hz), 124.0 (q, J=272.2 Hz), 129.4, 133.1, 134.4 (q, J=2.9 Hz), 146.0 (q, J=3.7 Hz), 153.2, 157.7, 159.8, 171.8.

HRMS (ESI) calcd for C₁₇H₁₇F₃N₅O₂S [M+H]⁺ 412.1050, found 412.1056.

### Example 6.12 N-(2-Amino-1-(4-(hydroxymethyl)thiazol-2-yl)-2-methylpropyl)-5-(5-(trifluoromethyl)pyridin-2-yl)-1H-pyrrole-2-carboxamide (23 & 24)

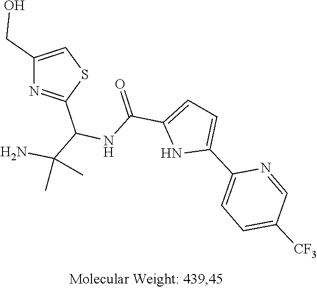 [SMILES: CC(C)(N)C(NC(=O)c1ccc(-c2ccc(C(F)(F)F)cn2)[nH]1)c1nc(CO)cs1]

Compounds 23 and 24 were obtained following the general procedure H and I from amine S44 and acid S4d. Compounds were purified using column chromatography on silica gel. Eluent CHCl₃-MeOH saturated with NH₃ (20:1 and 10:1).

23; (fR): M=321 mg. Yield=26% (over two steps). rt=1.248 min. Purity=100%. LC-MS: m/z [M+H]⁺=440 Da.

24; (fS): M=208 mg. Yield=17% (over two steps). rt=1.234 min. Purity=100%. LC-MS: m/z [M+H]⁺=440 Da.

¹H NMR (DMSO-d₆, 400 MHz): δ=1.08 (s, 3H), 1.13 (s, 3H), 4.57 (s, 2H), 5.10-5.50 (m, 2H), 6.96 (d, J=3.9 Hz, 1H), 7.05 (d, J=3.9 Hz, 1H), 7.32 (s, 1H), 8.09 (d, J=8.4 Hz, 1H), 8.18 (dd, J=8.6, 2.1 Hz, 1H), 8.65 (d, J=7.2 Hz, 1H), 8.87-8.91 (m, 1H).

¹³C NMR (DMSO-d₆, 100 MHz): δ=27.5, 28.3, 52.8, 59.4, 59.8, 111.2, 114.4, 115.1, 119.1, 122.4 (q, J=32.2 Hz), 124.0 (q, J=271.6 Hz), 129.3, 133.2, 134.5 (q, J=3.2 Hz), 145.9 (q, J=4.1 Hz), 153.3, 157.3, 159.4, 169.9.

HRMS (ESI) calcd for C₁₉H₂₁F₃N₅O₂S [M+H]⁺ 440.1363, found 440.1359.

### Example 6.13 N-(2-Amino-1-(4-(2-hydroxyethyl)thiazol-2-yl)ethyl)-5-(5-(trifluoromethyl)pyridin-2-yl)-1H-pyrrole-2-carboxamide (25 & 26)

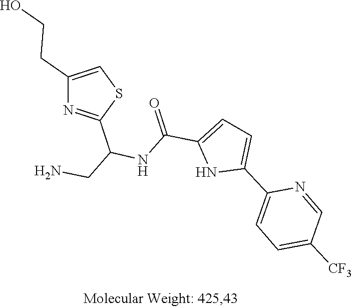 [SMILES: NCC(NC(=O)c1ccc(-c2ccc(C(F)(F)F)cn2)[nH]1)c1nc(CCO)cs1]

Compounds and 25 and 26 were obtained following the general procedure H and/from amine S41 and acid S4d. Compounds were purified using column chromatography on silica gel. Eluent CHCl₃-MeOH saturated with NH₃ (10:1 and 5:1).

25; (fR): M=490 mg. Yield=43% (over two steps). rt=1.198 min. Purity=100%. LC-MS: m/z [M+H]⁺=426 Da.

26; (fS): M=368 mg. Yield=32% (over two steps). rt=1.153 min. Purity=98%. LC-MS: m/z [M+H]⁺=426 Da.

¹H NMR (DMSO-d₆, 400 MHz): δ=2.85 (t, J=6.9 Hz, 2H), 3.01 (dd, J=13.2, 7.8 Hz, 1H), 3.15 (dd, J=13.2, 5.1 Hz, 1H), 3.71 (t, J=6.8 Hz, 2H), 4.69 (br. s., 1H), 5.19-5.27 (m, 1H), 6.99 (d, J=3.8 Hz, 1H), 7.05 (d, J=3.8 Hz, 1H), 7.18 (s, 1H), 8.12 (d, J=8.4 Hz, 1H), 8.19 (dd, J=8.4, 1.7 Hz, 1H), 8.89 (s, 2H).

¹³C NMR (DMSO-d₆, 100 MHz): δ=34.9, 46.0, 54.6, 60.2, 111.2, 114.1, 114.3, 118.9, 122.3 (q, J=32.4 Hz), 124.0 (q, J=271.8 Hz), 129.4, 133.1, 134.4 (q, J=3.3 Hz), 146.0 (q, J=4.2 Hz), 153.2 (q, J=1.1 Hz), 154.1, 159.9, 171.3.

HRMS (ESI) calcd for C₁₈H₁₉F₃N₅O₂S [M+H]⁺ 426.1206, found 426.1204.

### Example 6.14 N-(2-Amino-1-(4-(3-hydroxypropyl)thiazol-2-yl)ethyl)-5-(5-(trifluoromethyl)pyridin-2-yl)-1H-pyrrole-2-carboxamide (27 & 28)

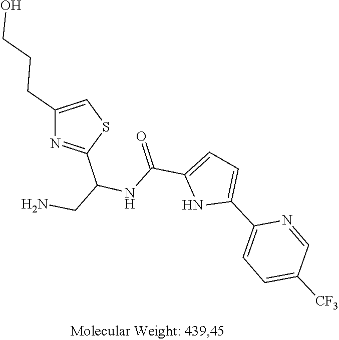 [SMILES: NCC(NC(=O)c1ccc(-c2ccc(C(F)(F)F)cn2)[nH]1)c1nc(CCCO)cs1]

Compounds 27 and 28 were obtained following the general procedure H and I from amine S42 and acid S4d. Compounds were purified using column chromatography on silica gel. Eluent CHCl₃-MeOH saturated with NH₃ (10:1 and 5:1).

27; (fR): M=483 mg. Yield=34% (over two steps). rt=1.226 min. Purity=100%. LC-MS: m/z [M+H]⁺=440 Da.

28; (fS): M=727 mg. Yield=39% (over two steps). rt=1.220 min. Purity=100%. LC-MS: m/z [M+H]⁺=440 Da.

¹H NMR (DMSO-d₆, 400 MHz): δ=1.73-1.83 (m, 2H), 2.71 (t, J=7.7 Hz, 2H), 2.99 (dd, J=13.2, 7.9 Hz, 1H), 3.13 (dd, J=13.2, 5.1 Hz, 1H), 3.44 (t, J=6.3 Hz, 2H), 4.50 (br. s., 1H), 5.17-5.24 (m, 1H), 6.98 (d, J=3.9 Hz, 1H), 7.06 (d, J=3.9 Hz, 1H), 7.14 (s, 1H), 8.13 (d, J=8.5 Hz, 1H), 8.21 (dd, J=8.5, 2.1 Hz, 1H), 8.84-8.94 (m, 2H).

¹³C NMR (DMSO-d₆, 100 MHz): δ=27.6, 32.1, 46.0, 54.6, 60.2, 111.2, 113.1, 114.3, 118.9, 122.3 (q, J=32.4 Hz), 124.0 (q, J=271.8 Hz), 129.4, 133.1, 134.4 (q, J=3.1 Hz), 146.0 (q, J=4.2 Hz), 153.2, 156.6, 159.9, 171.5.

HRMS (ESI) calcd for C₁₉H₂₁F₃N₅O₂S [M+H]⁺ 440.1363, found 440.1377.

### Example 6.15 N-(2-Amino-1-(4-(1,2-dihydroxyethyl)thiazol-2-yl)ethyl)-5-(5-(trifluoromethyl)pyridin-2-yl)-1H-pyrrole-2-carboxamide (29 & 30)

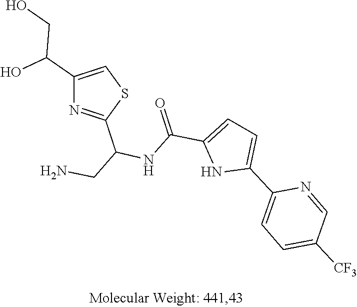 [SMILES: NCC(NC(=O)c1ccc(-c2ccc(C(F)(F)F)cn2)[nH]1)c1nc(C(O)CO)cs1]

Compounds 29 and 30 were obtained from amine S32 and acid S4d, following in sequence, the general procedure H, I and the TBDPS cleavage (below). Compounds were purified using column chromatography on silica gel. Eluent CHCl₃-MeOH saturated with NH₃ (10:1 and 5:1).

TBDPS cleavage: To a solution of TBDPS-protected compound (1 equiv) in THF (0.1 M), solution of TBAF trihydrate (1.1 equiv) in THF (0.1M) was added in a one portion. The mixture was stirred for 1-2 h at room temperature (TLC-control) and concentrated. Purification by flash chromatography using CHCl₃-MeOH saturated with NH₃ mixture (5:1 and 3:1) as eluent.

Note that compounds 29 & 30 were obtained as diastereisomeric mixture of 2 single compounds having the absolute configuration of the chiral carbon a as fR for 29 and fS for 30.

29; (fR): M=358 mg. Yield=29% (over two steps). rt=1.143 min. Purity=100%. LC-MS: m/z [M+H]⁺=442 Da.

30; (fS): M=238 mg. Yield=19% (over two steps). rt=1.124 min. Purity=100%. LC-MS: m/z [M+H]⁺=442 Da.

¹H NMR (DMSO-d₆, 400 MHz): δ=3.00 (ddd, J=13.2, 7.8, 2.1 Hz, 1H), 3.10-3.18 (m, 1H), 3.49 (ddd, J=10.9, 7.1, 2.0 Hz, 1H), 3.71 (dt, J=10.9, 4.1 Hz, 1H), 4.65 (dd, J=6.8, 4.2 Hz, 1H), 4.72 (br. s., 1H), 5.16-5.26 (m, 1H), 5.35 (br. s., 1H), 6.98 (d, J=3.9 Hz, 1H), 7.05 (d, J=3.9 Hz, 1H), 7.29-7.32 (m, 1H), 8.13 (d, J=8.4 Hz, 1H), 8.20 (dd, J=8.6, 2.1 Hz, 1H), 8.84-8.94 (m, 2H).

¹³C NMR (DMSO-d₆, 100 MHz): δ=45.7, 54.3, (65.8, 65.9), (71.3, 71.4), 111.3, 114.3, (114.5, 114.5), 118.9, 121.9 (q, J=32.4 Hz), 124.0 (q, J=271.8 Hz), 129.4, 133.2, 134.5 (q, J=3.3 Hz), 146.0 (q, J=4.2 Hz), 153.2 (q, J=1.1 Hz), (158.4, 158.5), (159.9, 159.9), (171.5, 171.5).

HRMS (ESI) calcd for C₁₈H₁₉F₃N₅O₃S [M+H]⁺ 442.1155, found 442.1172.

### Example 6.16 N-(1-Amino-2-(4-(hydroxymethyl)thiazol-2-yl)propan-2-yl)-3-methyl-5-(5-(trifluoromethyl)-pyridin-2-yl)-1H-pyrrole-2-carboxamide (31)

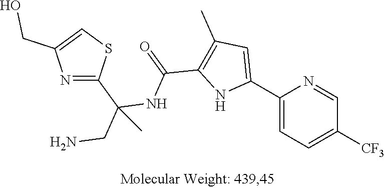 [SMILES: Cc1cc(-c2ccc(C(F)(F)F)cn2)[nH]c1C(=O)NC(C)(CN)c1nc(CO)cs1]

Compound 31 (racemate) was obtained following the general procedure H and I from amine S37 and the sodium salt S15. Compound was purified using column chromatography on silica gel. Eluent CHCl₃-MeOH saturated with NH₃ (20:1 and 10:1).

M=190 mg. Yield=12%. rt=1.271 min. Purity=100%. LC-MS: m/z [M+H]⁺=440 Da.

¹H NMR (DMSO-d₆, 400 MHz): δ=1.74 (s, 3H), 2.25 (s, 3H), 2.92 (d, J=13.2 Hz, 1H), 3.12 (d, J=13.2 Hz, 1H), 4.51 (d, J=5.1 Hz, 2H), 5.26 (t, J=5.7 Hz, 1H), 6.88 (s, 1H), 7.21-7.23 (m, 1H), 7.99 (d, J=8.4 Hz, 1H), 8.18 (dd, J=8.6, 2.3 Hz, 1H), 8.28 (s, 1H), 8.86-8.89 (m, 1H), 12.02 (br. s., 1H).

¹³C NMR (DMSO-d₆, 100 MHz): δ=13.1, 23.5, 51.8, 59.9, 60.5, 113.5, 113.8, 119.0, 122.2 (q, J=32.3 Hz), 122.6, 125.4, 126.5, 130.6, 134.4 (q, J=3.3 Hz), 145.8 (q, J=4.1 Hz), 153.2 (q, J=1.3 Hz), 156.8, 160.5, 176.6.

HRMS (ESI) calcd for C₁₈H₁₉F₃N₅O₂S [M+H]⁺ 440.1363, found 440.1373.

### Example 6.17 N-(1-Amino-2-(4-(hydroxymethyl)thiazol-2-yl)propan-2-yl)-5-(5-(trifluoromethyl)pyridin-2-yl)-1H-pyrrole-2-carboxamide (32)

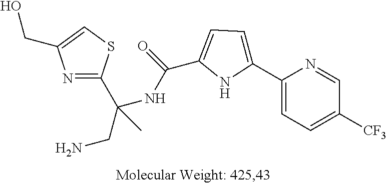 [SMILES: CC(CN)(NC(=O)c1ccc(-c2ccc(C(F)(F)F)cn2)[nH]1)c1nc(CO)cs1]

Compound 32 (racemate) was obtained following the general procedure H and I from amine S37 and acid S4d. Compound was purified using column chromatography on silica gel. Eluent CHCl₃/MeOH saturated with NH₃ (10:1 and 5:1).

M=332 mg. Yield=38%. rt=1.205 min. Purity=100%. LC-MS: m/z [M+H]⁺=426 Da.

¹H NMR (DMSO-d₆, 400 MHz): δ=1.76 (s, 3H), 2.94 (d, J=13.3 Hz, 1H), 3.14 (d, J=13.3 Hz, 1H), 4.52 (s, 2H), 5.29 (br. s., 1H), 6.90 (d, J=3.9 Hz, 1H), 7.04 (d, J=3.9 Hz, 1H), 7.23 (s, 1H), 8.10 (d, J=8.4 Hz, 1H), 8.19 (dd, J=8.6, 2.2 Hz, 1H), 8.40 (s, 1H), 8.85-8.92 (m, 1H).

¹³C NMR (DMSO-d₆, 100 MHz): δ=23.5, 51.7, 59.9, 60.6, 111.2, 113.8, 114.3, 118.9, 122.3 (q, J=32.3 Hz), 124.0 (q, J=271.8 Hz), 130.0, 132.9, 134.4 (q, J=3.1 Hz), 145.9 (q, J=4.2 Hz), 153.3, 156.9, 159.6, 176.4.

HRMS (ESI) calcd for C₁₈H₁₉F₃N₅O₂S [M+H]⁺ 426.1206, found 426.1217.

### Example 6.18 N-(2-Amino-1-(4-(hydroxymethyl)thiazol-2-yl)ethyl)-3-methyl-5-(5-(trifluoromethyl)pyridin-2-yl)-1H-pyrrole-2-carboxamide (33 & 34)

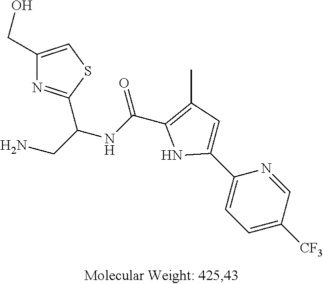 [SMILES: Cc1cc(-c2ccc(C(F)(F)F)cn2)[nH]c1C(=O)NC(CN)c1nc(CO)cs1]

Compounds 33 and 34 were obtained following the general procedure H and I from amine S38 and sodium salt S15. Compounds were purified using column chromatography on silica gel. Eluent CHCl₃-MeOH saturated with NH₃ (10:1 and 5:1).

33; (fR): M=248 mg. Yield=33% (over two steps). rt=1.333 min. Purity=100%. LC-MS: m/z [M+H]⁺=426 Da.

34; (fS): M=162 mg. Yield=22% (over two steps). rt=1.328 min. Purity=100%. LC-MS: m/z [M+H]⁺=426 Da.

¹H NMR (DMSO-d₆, 400 MHz): δ=2.33 (s, 3H), 3.00 (dd, J=13.2, 7.7 Hz, 1H), 3.12 (dd, J=13.3, 5.1 Hz, 1H), 4.55 (s, 2H), 5.18-5.26 (m, 1H), 5.34 (br. s., 1H), 6.90 (s, 1H), 7.29 (s, 1H), 7.98 (d, J=8.6 Hz, 1H), 8.17 (dd, J=8.6, 2.2 Hz, 1H), 8.81 (d, J=6.8 Hz, 1H), 8.86-8.90 (m, 1H).

¹³C NMR (DMSO-d₆, 100 MHz): δ=13.1, 46.1, 54.4, 59.8, 113.4, 114.0, 119.0, 122.3 (q, J=32.2 Hz), 124.0 (q, J=272.1 Hz), 124.6, 127.3, 130.9, 134.4 (q, J=3.2 Hz), 145.8 (q, J=4.1 Hz), 153.1, 157.7, 160.7, 172.2.

HRMS (ESI) calcd for C₁₈H₁₉F₃N₅O₂S [M+H]⁺ 426.1206, found 426.1213.

### Example 6.19 N-(2-Amino-1-(4,5-bis(hydroxymethyl)thiazol-2-yl)ethyl)-5-(5-(trifluoromethyl)pyridin-2-yl)-1H-pyrrole-2-carboxamide (35 & 36)

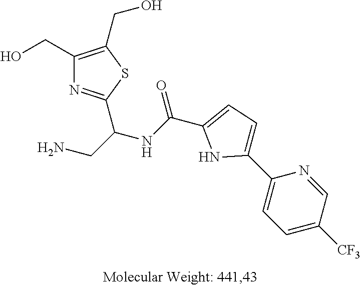 [SMILES: NCC(NC(=O)c1ccc(-c2ccc(C(F)(F)F)cn2)[nH]1)c1nc(CO)c(CO)s1]

Compounds 35 and 36 were obtained following the general procedure H and I from amine S40 and acid S4d. Compounds were purified using column chromatography on silica gel. Eluent CHCl₃-MeOH saturated with NH₃ (15:1 and 3:1).

35; (fR): M=468 mg. Yield=38% (over two steps). rt=1.221 min. Purity=100%. LC-MS: m/z [M+H]⁺=442 Da.

36; (fS): M=290 mg. Yield=24% (over two steps). rt=1.225 min. Purity=100%. LC-MS: m/z [M+H]⁺=442 Da.

¹H NMR (DMSO-d₆, 400 MHz): δ=2.99 (dd, J=13.1, 7.9 Hz, 1H), 3.07-3.15 (dd, J=13.2, 5.3 Hz, 1H), 4.46 (s, 2H), 4.65 (s, 2H), 5.08 (br. s., 1H), 5.12-5.19 (m, 1H), 5.45 (br. s., 1H), 6.97 (d, J=3.9 Hz, 1H), 7.05 (d, J=3.9 Hz, 1H), 8.13 (d, J=8.4 Hz, 1H), 8.20 (dd, J=8.6, 2.0 Hz, 1H), 8.84-8.91 (m, 1H).

¹³C NMR (DMSO-d₆, 100 MHz): δ=45.5, 54.1, 55.1, 57.4, 111.2, 114.3, 118.9, 122.3 (q, J=32.1 Hz), 129.4, 133.1, 134.4, 134.5, 136.3, 145.9, 146.0, 150.8, 153.2, 159.8, 169.0.

HRMS (ESI) calcd for C₁₈H₁₉F₃N₅O₃S [M+H]⁺ 442.1155, found 442.1151.

### Example 6.20 N-(2-Amino-1-(thiazol-2-yl)ethyl)-5-(5-(trifluoromethyl)pyridin-2-yl)-1H-pyrrole-2-carboxamide (37 & 38)

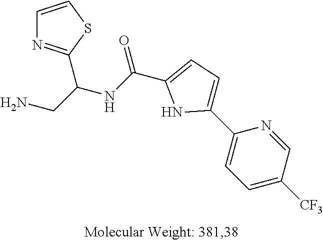 [SMILES: NCC(NC(=O)c1ccc(-c2ccc(C(F)(F)F)cn2)[nH]1)c1nccs1]

Compounds 37 and 38 were obtained following the general procedure H and I from amine S43 and acid S4d. Compounds were purified using column chromatography on silica gel. Eluent CHCl₃-MeOH saturated with NH₃ (10:1 and 5:1).

37; (fR): M=220 mg. Yield=30% (over two steps). rt=1.230 min. Purity=95%. LC-MS: m/z [M+H]⁺=382 Da.

38; (fS): M=342 mg. Yield=45% (over two steps). rt=1.236 min. Purity=96%. LC-MS: m/z [M+H]⁺=382 Da.

¹H NMR (DMSO-d₆, 400 MHz): δ=3.03 (dd, J=13.2, 7.8 Hz, 1H), 3.16 (dd, J=13.2, 5.3 Hz, 1H), 5.22-5.31 (m, 1H), 6.98 (d, J=3.8 Hz, 1H), 7.06 (d, J=3.8 Hz, 1H), 7.61 (d, J=3.2 Hz, 1H), 7.77 (d, J=3.2 Hz, 1H), 8.13 (d, J=8.5 Hz, 1H), 8.20 (dd, J=8.5, 2.0 Hz, 1H), 8.80-9.01 (m, 2H).

¹³C NMR (DMSO-d₆, 100 MHz): δ=45.9, 54.5, 111.2, 114.2, 118.9, 119.6, 122.3 (q, J=32.4 Hz), 124.0 (q, J=271.8 Hz), 129.3, 133.1, 134.4 (q, J=3.3 Hz), 142.4, 145.9 (q, J=4.1 Hz), 153.2 (q, J=1.3 Hz), 159.8, 172.1.

HRMS (ESI) calcd for C₁₆H₁₅F₃N₅OS [M+H]⁺ 382.0944, found 382.0952.

### Example 6.21 N-(2-Amino-1-(4-(hydroxymethyl)thiazol-2-yl)ethyl)-5-(6-chloropyridin-3-yl)-1H-pyrrole-2-carboxamide (39 & 40)

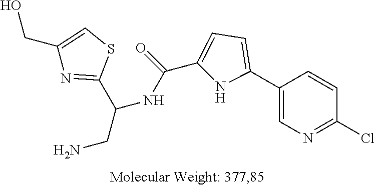 [SMILES: NCC(NC(=O)c1ccc(-c2ccc(Cl)nc2)[nH]1)c1nc(CO)cs1]

Compounds 39 and 40 were obtained following the general procedure H and I from amine S38 and acid S4e. Compounds were purified using column chromatography on silica gel. Eluent CHCl₃-MeOH saturated with NH₃ (10:1 and 5:1).

39; (fR): M=375 mg. Yield=24% (over two steps). rt=1.044 min. Purity=96%. LC-MS: m/z [M+H]⁺=378 Da.

40; (fS): M=387 mg. Yield=28% (over two steps). rt=0.999 min. Purity=100%. LC-MS: m/z [M+H]⁺=378 Da.

¹H NMR (DMSO-d₆, 400 MHz): δ=1.72 (br. s., 2H), 2.99 (dd, J=13.2, 7.9 Hz, 1H), 3.13 (dd, J=13.2, 5.3 Hz, 1H), 4.52 (s, 2H), 5.16-5.23 (m, 1H), 5.28 (br. s., 1H), 6.76 (d, J=3.9 Hz, 1H), 7.04 (d, J=3.9 Hz, 1H), 7.27 (s, 1H), 7.51 (d, J=8.5 Hz, 1H), 8.25 (dd, J=8.5, 2.6 Hz, 1H), 8.61 (d, J=8.1 Hz, 1H), 8.86 (d, J=2.4 Hz, 1H), 12.07 (br. s., 1H).

¹³C NMR (DMSO-d₆, 100 MHz): δ=45.5, 54.2, 59.8, 108.7, 112.8, 114.1, 124.2, 127.3, 128.3, 130.6, 135.3, 146.0, 147.8, 157.6, 160.4, 172.2.

HRMS (ESI) calcd for C₁₆H₁₇ClN₅O₂S [M+H]⁺ 378.0786, found 378.0786.

### Example 6.22 N-(2-Amino-1-(4-(hydroxymethyl)thiazol-2-yl)ethyl)-5-(6-(trifluoromethyl)pyridin-3-yl)-1H-pyrrole-2-carboxamide (41 & 42)

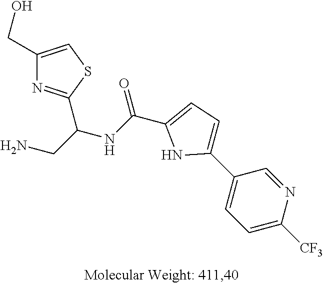 [SMILES: NCC(NC(=O)c1ccc(-c2ccc(C(F)(F)F)nc2)[nH]1)c1nc(CO)cs1]

Compounds 41 and 42 were obtained following the general procedure H and I from amine S38 and acid S4f. Compounds were purified using column chromatography on silica gel. Eluent CHCl₃-MeOH saturated with NH₃ (10:1 and 5:1).

41; (fR): M=240 mg. Yield=33% (over two steps). rt=1.246 min. Purity=100%. LC-MS: m/z [M+H]⁺=412 Da.

42; (fS): M=290 mg. Yield=40% (over two steps). rt=1.262 min. Purity=100%. LC-MS: m/z [M+H]⁺=412 Da.

¹H NMR (DMSO-d₆, 400 MHz): δ=3.03 (dd, J=13.2, 7.8 Hz, 1H), 3.16 (dd, J=13.2, 5.4 Hz, 1H), 4.55 (s, 2H), 5.20-5.28 (m, 1H), 5.36 (br. s., 1H), 6.93 (d, J=3.9 Hz, 1H), 7.11 (d, J=3.9 Hz, 1H), 7.30 (s, 1H), 7.88 (d, J=8.3 Hz, 1H), 8.47 (dd, J=8.3, 1.7 Hz, 1H), 8.71 (d, J=7.8 Hz, 1H), 9.22 (d, J=1.6 Hz, 1H).

¹³C NMR (DMSO-d₆, 100 MHz): δ=45.6, 54.4, 59.8, 109.9, 113.0, 114.2, 120.8 (q, J=2.6 Hz), 121.9 (q, J=273.5 Hz), 129.2, 130.4, 130.9, 132.9, 143.7 (q, J=33.9 Hz), 146.3, 157.7, 160.4, 172.1.

HRMS (ESI) calcd for C₁₇H₁₇F₃N₅O₂S [M+H]⁺ 412.1050, found 412.1054.

### Example 6.23 N-(2-Amino-1-(4-(hydroxymethyl)thiazol-2-yl)ethyl)-5-(6-(trifluoromethyl)pyridazin-3-yl)-1H-pyrrole-2-carboxamide (43 & 44)

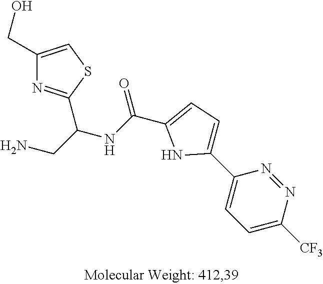 [SMILES: NCC(NC(=O)c1ccc(-c2ccc(C(F)(F)F)nn2)[nH]1)c1nc(CO)cs1]

Compounds 43 and 44 were obtained following the general procedure H and I from amine S38 and acid S4g. Compounds were purified using column chromatography on silica gel. Eluent CHCl₃-MeOH saturated with NH₃ (10:1 and 5:1).

43; (fR): M=322 mg. Yield=42% (over two steps). rt=1.095 min. Purity=100%. LC-MS: m/z [M+H]⁺=413 Da.

44; (fS): M=193 mg. Yield=25% (over two steps). rt=1.065 min. Purity=100%. LC-MS: m/z [M+H]⁺=413 Da.

¹H NMR (DMSO-d₆, 400 MHz): δ=3.01 (dd, J=13.1, 7.8 Hz, 1H), 3.14 (dd, J=13.2, 5.3 Hz, 1H), 4.55 (s, 2H), 5.20-5.28 (m, 1H), 5.32 (br. s., 1H), 7.03 (d, J=3.9 Hz, 1H), 7.22 (d, J=3.9 Hz, 1H), 7.30 (s, 1H), 8.24 (d, J=9.0 Hz, 1H), 8.48 (d, J=9.0 Hz, 1H), 8.97 (d, J=7.1 Hz, 1H).

¹³C NMR (DMSO-d₆, 100 MHz): δ=45.7, 54.3, 59.8, 112.7, 114.2, 114.4, 121.8 (q, J=273.7 Hz), 124.0, 125.2 (q, J=1.7 Hz), 130.1, 130.6, 148.1 (q, J=33.9 Hz), 154.5, 157.7, 159.7, 171.6.

HRMS (ESI) calcd for C₁₆H₁₆F₃N₆O₂S [M+H]⁺ 413.1002, found 413.1000.

### Example 6.24 N-(2-Amino-1-(4-(hydroxymethyl)thiazol-2-yl)ethyl)-5-(5-chloropyrimidin-2-yl)-1H-pyrrole-2-carboxamide (45 & 46)

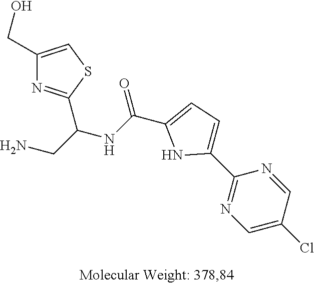 [SMILES: NCC(NC(=O)c1ccc(-c2ncc(Cl)cn2)[nH]1)c1nc(CO)cs1]

Compounds 45 and 46 were obtained following the general procedure H and I from amine S38 and acid S4h. Compounds were purified using column chromatography on silica gel. Eluent CHCl₃-MeOH saturated with NH₃ (10:1 and 5:1).

45; (fR): M=186 mg. Yield=24% (over two steps). rt=1.078 min. Purity=100%. LC-MS: m/z [M+H]⁺=379 Da.

46; (fS): M=124 mg. Yield=16% (over two steps). rt=1.099 min. Purity=100%. LC-MS: m/z [M+H]⁺=379 Da.

¹H NMR (DMSO-d₆, 400 MHz): δ=2.99 (dd, J=13.2, 7.8 Hz, 1H), 3.12 (dd, J=13.3, 5.3 Hz, 1H), 4.55 (s, 2H), 5.16-5.26 (m, 1H), 5.31 (br. s., 1H), 6.91 (d, J=3.8 Hz, 1H), 6.99 (d, J=3.8 Hz, 1H), 7.29 (s, 1H), 8.90 (s, 2H), 8.97 (d, J=7.2 Hz, 1H).

¹³C NMR (DMSO-d₆, 100 MHz): δ=46.1, 54.5, 59.8, 112.5, 114.1, 114.9, 127.3, 129.7, 132.2, 155.9 (2C), 156.3, 157.7, 159.5, 171.8.

HRMS (ESI) calcd for C₁₅H₁₆ClN₆O₂S [M+H]⁺ 379.0738, found 379.0745.

### Example 6.25 N-(2-Amino-1-(4-(hydroxymethyl)thiazol-2-yl)ethyl)-5-(2-methoxypyridin-4-yl)-1H-pyrrole-2-carboxamide (47 & 48)

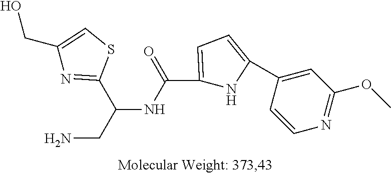 [SMILES: COc1cc(-c2ccc(C(=O)NC(CN)c3nc(CO)cs3)[nH]2)ccn1]

Compounds 47 and 48 were obtained following the general procedure H and I from amine S38 and acid S8. Compounds were purified using column chromatography on silica gel. Eluent CHCl₃-MeOH saturated with NH₃ (20:1 and 10:1).

47; (fR): M=485 mg. Yield=32% (over two steps). rt=0.838 min. Purity=100%. LC-MS: m/z [M+H]⁺=374 Da.

48; (fS): M=423 mg. Yield=28% (over two steps). rt=0.839 min. Purity=100%. LC-MS: m/z [M+H]⁺=374 Da.

¹H NMR (DMSO-d₆, 400 MHz): δ=1.74 (br. s., 2H), 3.00 (dd, J=13.1, 7.9 Hz, 1H), 3.14 (dd, J=13.2, 5.3 Hz, 1H), 3.85 (s, 3H), 4.54 (s, 2H), 5.18-5.25 (m, 1H), 5.30 (br. s., 1H), 6.87 (d, J=3.9 Hz, 1H), 7.03 (d, J=3.9 Hz, 1H), 7.29 (t, J=1.0 Hz, 1H), 7.30 (d, J=0.9 Hz, 1H), 7.41 (dd, J=5.5, 1.4 Hz, 1H), 8.10 (d, J=5.4 Hz, 1H), 8.66 (d, J=7.9 Hz, 1H), 12.07 (br. s., 1H).

¹³C NMR (DMSO-d₆, 100 MHz): δ=45.7, 53.2, 54.5, 59.8, 104.5, 109.8, 112.9, 113.1, 114.1, 128.7, 132.1, 141.5, 147.2, 157.6, 160.4, 164.5, 172.2.

HRMS (ESI) calcd for C₁₇H₂₀N₅O₃S [M+H]⁺ 374.1281, found 374.1281.

### List of Particular Embodiments

The following listing of embodiments is illustrative of the variety of embodiments with respect to breadth, combinations and sub-combinations, class of invention, etc., elucidated herein, but is not intended to be an exhaustive enumeration of all embodiments finding support herein.

Embodiment 1. A compound of Formula (I):

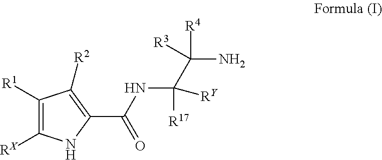

or a pharmaceutically acceptable salt thereof,
wherein in some embodiments:
each of R¹, R², R³, and R⁴, is, independently, H or C₁₋₃ alkyl;
R¹⁷ is H or CH₃;

### R^{X} is

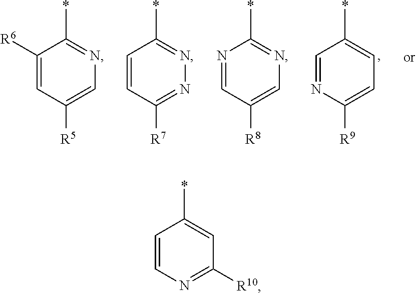

wherein * indicates the point of attachment to the pyrrolyl ring,
each of R⁵, R⁷, R⁸, and R⁹ is H, CH₃, halogen, or CF₃;
R⁶ is H or halogen; and
R¹⁰ is H, halogen, CF₃, OCH₃ or OCF₃; and

### R^{Y} is

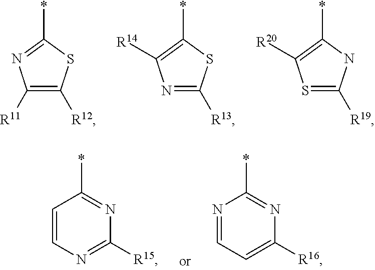

wherein * indicates the point of attachment to the Formula I backbone; and
each of R¹¹, R¹², R¹³, R¹⁴, R¹⁵, R¹⁶, R¹⁹, and R²⁰, is, independently, H, CH₂OH, CH₂CH₂OH, (CH₂)₃OH, or CH(OH)CH₂OH.

Embodiment 2. A compound of Formula II):

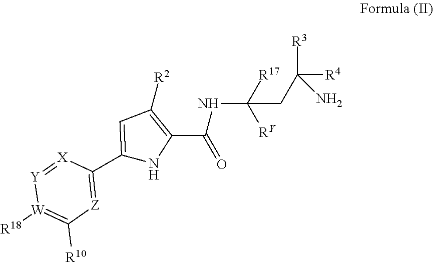

or pharmaceutically acceptable salt thereof,
wherein in some embodiments:

### W is C or N;

X, Y, and Z are CH or N, with the caveats that 1) only X and Y, or X and Z, are both N in any compound, and 2) that at least one of W, X, Y, or Z is N;
each of R², R³, and R⁴, is, independently, H or CH₃;
R¹⁰ is H when X is N, and is H or OCH₃ when X is CH;

### R^{Y} is

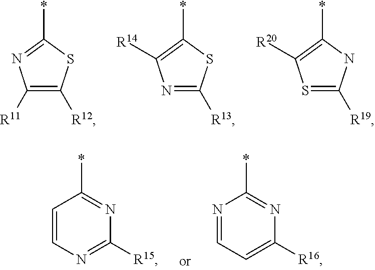

wherein * indicates the point of attachment to the Formula II backbone; and
each of R¹¹, R¹², R¹³, R¹⁴, R¹⁵, R¹⁶, R¹⁹, and R²⁰, is, independently, H, CH₂OH, CH₂CH₂OH, (CH₂)₃OH, or CH(OH)CH₂OH;
R¹⁷ is H or CH₃; and
R¹⁸ is absent when W is N, and is H, CH₃, Cl, F, CH₂F, CHF₂, or CF₃ when W is C.

Embodiment 3. The compound or pharmaceutically acceptable salt of Embodiment 1 or 2 wherein R¹ is H.

Embodiment 4. The compound or pharmaceutically acceptable salt of Embodiments 1 or 2, wherein R¹ is C₁₋₃ alkyl.

Embodiment 5. The compound or pharmaceutically acceptable salt of any one of Embodiments 1-4, wherein R² is H.

Embodiment 6. The compound or pharmaceutically acceptable salt of any one of Embodiments 1-4, wherein R² is C₁₋₃ alkyl.

Embodiment 7. The compound or pharmaceutically acceptable salt of any one of Embodiments 1-6, wherein R³ is H.

Embodiment 8. The compound or pharmaceutically acceptable salt of any one of Embodiments 1-6, wherein R³ is C₁₋₃ alkyl.

Embodiment 9. The compound or pharmaceutically acceptable salt of any one of Embodiments 1-8, wherein R⁴ is H.

Embodiment 10. The compound or pharmaceutically acceptable salt of any one of Embodiments 1-8, wherein R⁴ is C₁₋₃ alkyl.

Embodiment 11. The compound or pharmaceutically acceptable salt of any one of Embodiments 1-10, wherein R¹⁷ is H.

Embodiment 12. The compound or pharmaceutically acceptable salt of any one of Embodiments 1-10, wherein R¹⁷ is CH₃.

Embodiment 13. The compound or pharmaceutically acceptable salt of any one of Embodiments 1-12, wherein R^{x} is

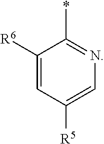

Embodiment 14. The compound or pharmaceutically acceptable salt of Embodiment 13, wherein R⁵ is H.

Embodiment 15. The compound or pharmaceutically acceptable salt of Embodiment 13, wherein R⁵ is CH₃.

Embodiment 16. The compound or pharmaceutically acceptable salt of Embodiment 13 wherein R⁵ is CF₃.

Embodiment 17. The compound or pharmaceutically acceptable salt of Embodiment 13, wherein R⁵ is halogen.

Embodiment 18. The compound or pharmaceutically acceptable salt of any one of Embodiments 13-17, wherein R⁶ is H.

Embodiment 19. The compound or pharmaceutically acceptable salt of any one of Embodiments 13-17, wherein R⁶ is halogen.

Embodiment 20. The compound or pharmaceutically acceptable salt of any one of Embodiments 1-12, wherein R^{x} is

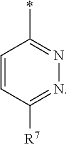

Embodiment 21. The compound or pharmaceutically acceptable salt of Embodiment 20, wherein R⁷ is H.

Embodiment 22. The compound or pharmaceutically acceptable salt of Embodiment 20, wherein R⁷ is CH₃.

Embodiment 23. The compound or pharmaceutically acceptable salt of Embodiment 20, wherein R⁷ is CF₃.

Embodiment 24. The compound or pharmaceutically acceptable salt of Embodiment 20, wherein R⁷ is halogen.

Embodiment 25. The compound or pharmaceutically acceptable salt of any one of Embodiments 1-12, wherein R^{x} is

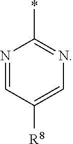

Embodiment 26. The compound or pharmaceutically acceptable salt of Embodiment 25, wherein R⁸ is H.

Embodiment 27. The compound or pharmaceutically acceptable salt of Embodiment 25, wherein R⁸ is CH₃.

Embodiment 28. The compound or pharmaceutically acceptable salt of Embodiment 25, wherein R⁸ is CF₃.

Embodiment 29. The compound or pharmaceutically acceptable salt of Embodiment 25, wherein R⁸ is halogen.

Embodiment 30. The compound or pharmaceutically acceptable salt of any one of Embodiments 1-12, wherein R^{x} is

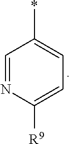

Embodiment 31. The compound or pharmaceutically acceptable salt of Embodiment 30, wherein R⁹ is H.

Embodiment 32. The compound or pharmaceutically acceptable salt of Embodiment 30, wherein R⁹ is CH₃.

Embodiment 33. The compound or pharmaceutically acceptable salt of Embodiment 30, wherein R⁹ is CF₃.

Embodiment 34. The compound or pharmaceutically acceptable salt of Embodiment 30, wherein R⁹ is halogen.

Embodiment 35. The compound or pharmaceutically acceptable salt of any one of Embodiments 1-12, wherein R^{x} is

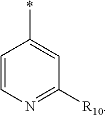

Embodiment 36. The compound or pharmaceutically acceptable salt of Embodiment 35, wherein R¹⁰ is H.

Embodiment 37. The compound or pharmaceutically acceptable salt of Embodiment 35, wherein R¹⁰ is OCH₃.

Embodiment 38. The compound or pharmaceutically acceptable salt of Embodiment 35, wherein R¹⁰ is CF₃.

Embodiment 39. The compound or pharmaceutically acceptable salt of Embodiment 35, wherein R¹⁰ is OCF₃.

Embodiment 40. The compound or pharmaceutically acceptable salt of Embodiment 35, wherein R¹⁰ is halogen.

Embodiment 41. The compound or pharmaceutically acceptable salt of any one of Embodiments 1-40, wherein R^{Y} is

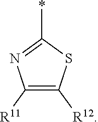

Embodiment 42. The compound or pharmaceutically acceptable salt of Embodiment 41, wherein R¹¹ is H.

Embodiment 43. The compound or pharmaceutically acceptable salt of Embodiment 41, wherein R¹¹ is CH₂OH.

Embodiment 44. The compound or pharmaceutically acceptable salt of Embodiment 41, wherein R¹¹ is CH₂CH₂OH.

Embodiment 45. The compound or pharmaceutically acceptable salt of Embodiment 41, wherein R¹¹ is (CH₂)₃OH.

Embodiment 46. The compound or pharmaceutically acceptable salt of Embodiment 41, wherein R¹¹ is CH(OH)CH₂OH.

Embodiment 47. The compound or pharmaceutically acceptable salt of any one of Embodiments 41-46, wherein R¹² is H.

Embodiment 48. The compound or pharmaceutically acceptable salt of any one of Embodiments 41-46, wherein R¹² is CH₂OH.

Embodiment 49. The compound or pharmaceutically acceptable salt of any one of Embodiments 41-46, wherein R¹² is CH₂CH₂OH.

Embodiment 50. The compound or pharmaceutically acceptable salt of any one of Embodiments 41-46, wherein R¹² is (CH₂)₃OH.

Embodiment 51. The compound or pharmaceutically acceptable salt of any one of Embodiments 41-46, wherein R¹² is CH(OH)CH₂OH.

Embodiment 52. The compound or pharmaceutically acceptable salt of any one of Embodiments 1-40, wherein R^{Y} is

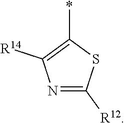

Embodiment 53. The compound or pharmaceutically acceptable salt of Embodiment 52, wherein R¹³ is H.

Embodiment 54. The compound or pharmaceutically acceptable salt of Embodiment 52, wherein R¹³ is CH₂OH.

Embodiment 55. The compound or pharmaceutically acceptable salt of Embodiment 52 wherein R¹³ is CH₂CH₂OH.

Embodiment 56. The compound or pharmaceutically acceptable salt of Embodiment 52, wherein R¹³ is (CH₂)₃OH.

Embodiment 57. The compound or pharmaceutically acceptable salt of Embodiment 52, wherein R¹³ is CH(OH)CH₂OH.

Embodiment 58. The compound or pharmaceutically acceptable salt of any one of Embodiments 52-57, wherein R¹⁴ is H.

Embodiment 59. The compound or pharmaceutically acceptable salt of any one of Embodiments 52-57, wherein R¹⁴ is CH₂OH.

Embodiment 60. The compound or pharmaceutically acceptable salt of any one of Embodiments 52-57, wherein R¹⁴ is CH₂CH₂OH.

Embodiment 61. The compound or pharmaceutically acceptable salt of any one of Embodiments 52-57, wherein R¹⁴ is (CH₂)₃OH.

Embodiment 62. The compound or pharmaceutically acceptable salt of any one of Embodiments 52-57, wherein R¹⁴ is CH(OH)CH₂OH.

Embodiment 63. The compound or pharmaceutically acceptable salt of any one of Embodiments 1-40, wherein R^{Y} is

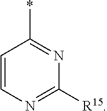

Embodiment 64. The compound or pharmaceutically acceptable salt of Embodiment 63, wherein R¹⁵ is H.

Embodiment 65. The compound or pharmaceutically acceptable salt of Embodiment 63, wherein R¹⁵ is CH₂OH.

Embodiment 66. The compound or pharmaceutically acceptable salt of Embodiment 63, wherein R¹⁵ is CH₂CH₂OH.

Embodiment 67. The compound or pharmaceutically acceptable salt of Embodiment 63, wherein R¹⁵ is (CH₂)₃OH.

Embodiment 68. The compound or pharmaceutically acceptable salt of Embodiment 63, wherein R¹⁵ is CH(OH)CH₂OH.

Embodiment 69. The compound or pharmaceutically acceptable salt of any one of Embodiments 1-40, wherein R^{Y} is

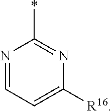

Embodiment 70. The compound or pharmaceutically acceptable salt of Embodiment 69, wherein R¹⁶ is H.

Embodiment 71. The compound or pharmaceutically acceptable salt of Embodiment 69, wherein R¹⁶ is CH₂OH.

Embodiment 72. The compound or pharmaceutically acceptable salt of Embodiment 69, wherein R¹⁶ is CH₂CH₂OH.

Embodiment 73. The compound or pharmaceutically acceptable salt of Embodiment 69, wherein R¹⁵ is (CH₂)₃OH.

Embodiment 74. The compound or pharmaceutically acceptable salt of Embodiment 69, wherein R¹⁶ is CH(OH)CH₂OH.

Embodiment 75. The compound or pharmaceutically acceptable salt of any one of Embodiments 1-40, wherein R^{Y} is

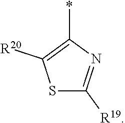

Embodiment 76. The compound or pharmaceutically acceptable salt of Embodiment 75, wherein R¹⁹ is H.

Embodiment 77. The compound or pharmaceutically acceptable salt of Embodiment 75, wherein R¹⁹ is CH₂OH.

Embodiment 78. The compound or pharmaceutically acceptable salt of Embodiment 75 wherein R¹⁹ is CH₂CH₂OH.

Embodiment 79. The compound or pharmaceutically acceptable salt of Embodiment 75, wherein R¹⁹ is (CH₂)₃OH.

Embodiment 80. The compound or pharmaceutically acceptable salt of Embodiment 75, wherein R¹⁹ is CH(OH)CH₂OH.

Embodiment 81. The compound or pharmaceutically acceptable salt of any one of Embodiments 75-80, wherein R²⁰ is H.

Embodiment 82. The compound or pharmaceutically acceptable salt of any one of Embodiments 75-80, wherein R²⁰ is CH₂OH.

Embodiment 83. The compound or pharmaceutically acceptable salt of any one of Embodiments 75-80, wherein R²⁰ is CH₂CH₂OH.

Embodiment 84. The compound or pharmaceutically acceptable salt of any one of Embodiments 75-80, wherein R²⁰ is (CH₂)₃OH.

Embodiment 85. The compound or pharmaceutically acceptable salt of any one of Embodiments 75-80, wherein R²⁰ is CH(OH)CH₂OH.

Embodiment 86. A compound of Formula III):

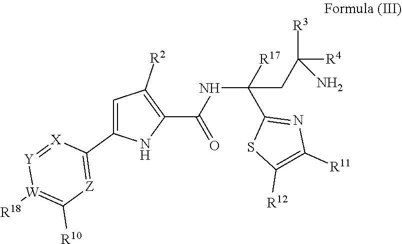

or pharmaceutically acceptable salt thereof,
wherein in some embodiments:

### W is C or N;

X, Y, and Z are CH or N, with the caveats that 1) only X and Y, or X and Z, are both N in any compound, and 2) that at least one of W, X, Y, or Z is N;
each of R², R³, and R⁴, is, independently, H or CH₃;
R¹⁰ is H when X is N, and is H or OCH₃ when X is CH;
Each of R¹¹ and R¹² is, independently, H, CH₂OH, (CH₂)₂OH, (CH₂)₃OH or CH(OH)CH₂OH;
R¹⁷ is H or CH₃; and
R¹⁸ is absent when W is N, and is H, CH₃, Cl, F, CH₂F, CHF₂, or CF₃ when W is C.

Embodiment 87. The compound or pharmaceutically acceptable salt of Embodiment 86, wherein R² is H.

Embodiment 88. The compound or pharmaceutically acceptable salt of Embodiment 86, wherein R² is CH₃.

Embodiment 89. The compound or pharmaceutically acceptable salt of any one of Embodiments 86-88, wherein R³ is H.

Embodiment 90. The compound or pharmaceutically acceptable salt of any one of Embodiments 86-88, wherein R³ is CH₃.

Embodiment 91. The compound or pharmaceutically acceptable salt of any one of Embodiments 86-90, wherein R⁴ is H.

Embodiment 92. The compound or pharmaceutically acceptable salt of any one of Embodiments 86-90, wherein R⁴ is CH₃.

Embodiment 93. The compound or pharmaceutically acceptable salt of any one of Embodiments 86-92, wherein R¹¹ is H.

Embodiment 94. The compound or pharmaceutically acceptable salt of any one of Embodiments 86-92, wherein R¹¹ is CH₂OH.

Embodiment 95. The compound or pharmaceutically acceptable salt of any one of Embodiments 86-92, wherein R¹¹ is (CH₂)₂OH.

Embodiment 96. The compound or pharmaceutically acceptable salt of any one of Embodiments 86-92, wherein R¹¹ is (CH₂)₃OH

Embodiment 97. The compound or pharmaceutically acceptable salt of any one of Embodiments 86-92, wherein R¹¹ is CH(OH)CH₂OH.

Embodiment 98. The compound or pharmaceutically acceptable salt of any one of Embodiments 86-97, wherein R¹² is H.

Embodiment 99. The compound or pharmaceutically acceptable salt of any one of Embodiments 86-97, wherein R¹² is CH₂OH.

Embodiment 100. The compound or pharmaceutically acceptable salt of any one of Embodiments 86-97, wherein R¹² is (CH₂)₂OH.

Embodiment 101. The compound or pharmaceutically acceptable salt of any one of Embodiments 86-97, wherein R¹² is (CH₂)₃OH

Embodiment 102. The compound or pharmaceutically acceptable salt of any one of Embodiments 86-97, wherein R¹² is CH(OH)CH₂OH.

Embodiment 103. The compound or pharmaceutically acceptable salt of any one of Embodiments 86-102, wherein R¹⁷ is H.

Embodiment 104. The compound or pharmaceutically acceptable salt of any one of Embodiments 86-102, wherein R¹⁷ is CH₃.

Embodiment 105. The compound or pharmaceutically acceptable salt of any one of Embodiments 86-104, wherein W is N and R¹⁸ is absent.

Embodiment 106. The compound or pharmaceutically acceptable salt of any one of Embodiments 86-104, wherein W is C.

Embodiment 107. The compound or pharmaceutically acceptable salt of Embodiment 106, wherein R¹⁸ is H.

Embodiment 108. The compound or pharmaceutically acceptable salt of Embodiment 106, wherein R¹⁸ is CH₃.

Embodiment 109. The compound or pharmaceutically acceptable salt of Embodiment 106, wherein R¹⁸ is Cl.

Embodiment 110. The compound or pharmaceutically acceptable salt of Embodiment 106, wherein R¹⁸ is F.

Embodiment 111. The compound or pharmaceutically acceptable salt of Embodiment 106, wherein R¹⁸ is CH₂F.

Embodiment 112. The compound or pharmaceutically acceptable salt of Embodiment 106, wherein R¹⁸ is CHF₂.

Embodiment 113. The compound or pharmaceutically acceptable salt of Embodiment 106, wherein R¹⁸ is CF₃.

Embodiment 114. The compound or pharmaceutically acceptable salt of any one of Embodiments 86-113, wherein X in N.

Embodiment 115. The compound or pharmaceutically acceptable salt of any one of Embodiments 86-113, wherein only X is N, of W, X, Y, and Z, and R¹⁰ is H.

Embodiment 116. The compound or pharmaceutically acceptable salt of any one of Embodiments 86-113, wherein only X and Y are N, of W, X, Y, and Z, and R¹⁰ is H.

Embodiment 117. The compound or pharmaceutically acceptable salt of any one of Embodiments 86-113, wherein only X and Z are N, of W, X, Y, and Z, and R¹⁰ is H.

Embodiment 118. The compound or pharmaceutically acceptable salt of any one of Embodiments 86-113, wherein X in CH.

Embodiment 119. The compound or pharmaceutically acceptable salt of Embodiment 118, wherein R¹⁰ is H.

Embodiment 120. The compound or pharmaceutically acceptable salt of Embodiment 118, wherein R¹⁰ is OCH₃.

Embodiment 121. The compound or pharmaceutically acceptable salt of any one of Embodiments 1-120 wherein C₁₋₃ alkyl is methyl.

Embodiment 122. The compound or pharmaceutically acceptable salt of any one of Embodiments 1-120 wherein C₁₋₃ alkyl is ethyl.

Embodiment 123. The compound or pharmaceutically acceptable salt of any one of Embodiments 1-120 wherein C₁₋₃ alkyl is propyl.

Embodiment 124. The compound or pharmaceutically acceptable salt of any one of Embodiments 1-123 wherein halogen is Cl, F, Br, or I.

Embodiment 125. The compound or pharmaceutically acceptable salt of any one of Embodiments 1-123 wherein halogen is Cl, F, or I.

Embodiment 126. The compound or pharmaceutically acceptable salt of any one of Embodiments 1-123 wherein halogen is Cl or F.

Embodiment 127. The compound or pharmaceutically acceptable salt of any one of Embodiments 1-123 wherein halogen is Cl.

Embodiment 128. The compound or pharmaceutically acceptable salt of any one of Embodiments 1-123 wherein halogen is F.

Embodiment 129. The compound or pharmaceutically acceptable salt of any one of Embodiments 1-128, wherein the compound is an S enantiomer.

Embodiment 130. The compound or pharmaceutically acceptable salt of any one of Embodiments 1-128, wherein the compound is an R enantiomer.

Embodiment 131. The compound or pharmaceutically acceptable salt of any one of Embodiments 1-128, wherein the compound is a mixture of R and S enantiomers.

Embodiment 132. The compound of any one of Embodiments 1-131.

Embodiment 133. The pharmaceutically acceptable salt of any one of Embodiment 1-131.

Embodiment 134. A composition comprising the compound or pharmaceutically acceptable salt of any one of Embodiments 1-133.

Embodiment 135. The composition of Embodiment 134, further comprising one or more carriers, binders, fillers, vehicles, disintegrants, surfactants, dispersion or suspension aids, thickening or emulsifying agents, isotonic agents, preservatives, lubricants, or other excipient.

Embodiment 136. A method of inhibiting HIV in a person in need thereof comprising administering a therapeutically effective amount of the compound, pharmaceutically acceptable salt, or composition of any one of Embodiments 1-135.

Embodiment 137. A method of treating an HIV infection comprising administering a therapeutically effective amount of the compound, pharmaceutically acceptable salt, or composition of any one of Embodiment 1-135 to a person in need thereof.

Embodiment 138. A method of eradicating, reducing, or slowing an HIV infection comprising administering a therapeutically effective amount of the compound, pharmaceutically acceptable salt, or composition of any one of Embodiments 1-135 to a person in need thereof.

Embodiment 139. A method of reducing the viral load associated with an HIV infection comprising administering a therapeutically effective amount of the compound, pharmaceutically acceptable salt, or composition of any one of Embodiments 1-135 to a person in need thereof.

Embodiment 140. A method of reducing reoccurrence of an HIV infection comprising administering a therapeutically effective amount of the compound, pharmaceutically acceptable salt, or composition of any one of Embodiments 1-135 to a person in need thereof.

Embodiment 141. A method of reducing an adverse physiological impact of an HIV infection comprising administering a therapeutically effective amount of the compound, pharmaceutically acceptable salt, or composition of any one of Embodiments 1-135 to a person in need thereof.

Embodiment 142. A method of inducing remission of an organ injury from an HIV infection comprising administering a therapeutically effective amount of the compound, pharmaceutically acceptable salt, or composition of any one of Embodiments 1-135 to a person in need thereof.

Embodiment 143. A method of prophylactically treating an HIV infection in a subject in need thereof, wherein the subject is afflicted with a latent HIV infection, comprising administering to the subject a therapeutically effective amount of the compound, pharmaceutically acceptable salt, or composition of any one of Embodiments 1-135.

Embodiment 144. The compound, pharmaceutically acceptable salt, or composition of any one of Embodiments 1-135 for use in treating an HIV infection.

Embodiment 145. Use of the compound, pharmaceutically acceptable salt, or composition of any one of Embodiments 1-135 in the manufacture of a medicament for treating an HIV infection.

Embodiment 146. The method, or compound, pharmaceutically acceptable salt, or composition for use, or use of any one of Embodiments 136-145, where in HIV is HIV-1.

It should be manifest that each of Embodiments 136 and 138-143 can be modified in a manner similar to the modification of Embodiment 137 by Embodiments 144-146. Embodiments 3-85 and 87-133 illustrate how substituents and alternate forms encompassed by Formulae (I), (II), and (III) can be combined into particular species and subgenera. Not all possible alternatives are explicitly listed and other disclosed substituents may be combined in like manner. Similarly, alternative embodiments may be combined to form larger subgenera.

The foregoing description details specific methods and compositions that can be employed to make and use the compounds described herein, and represents the best mode contemplated. However, it is apparent for one of ordinary skill in the art that further compounds with the desired pharmacological properties can be prepared in an analogous manner, and that the disclosed compounds can also be obtained from different starting compounds via different chemical reactions. Similarly, different pharmaceutical compositions may be prepared and used with substantially the same result. Thus, however detailed the foregoing may appear in text, it should not be construed as limiting the scope of the claims.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1. A compound of Formula (I): 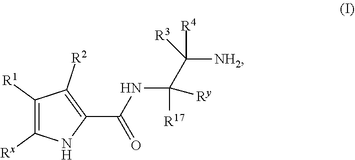
or a pharmaceutically acceptable salt thereof,
wherein:
each of R¹, R², R³, and R⁴, is, independently, H or C₁₋₃ alkyl;
R¹⁷ is H or CH₃;
R^{x} is 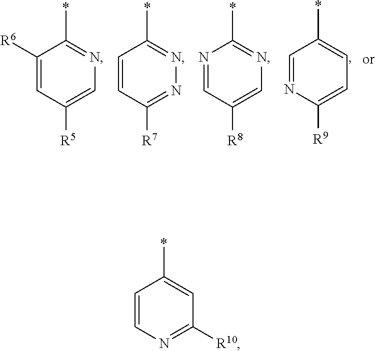
wherein * indicates the point of attachment;
each of R⁵, R⁷, R⁸, and R⁹ is H, CH₃, halogen, or CF₃;
R⁶ is H or halogen; and
R¹⁰ is H, halogen, CF₃, OCH₃ or OCF₃; and
R^{Y} is 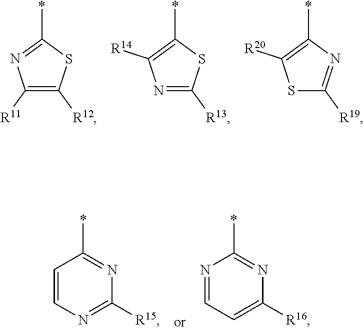
wherein * indicates the point of attachment: and
each of R¹¹, R¹², R¹³, R¹⁴, R¹⁵, R¹⁶, R¹⁹, and R²⁰, is, independently, H, CH₂OH, CH₂CH₂OH, (CH₂)₃OH or CH(OH)CH₂OH.

2. The compound of claim 1, having a structure of Formula (II): 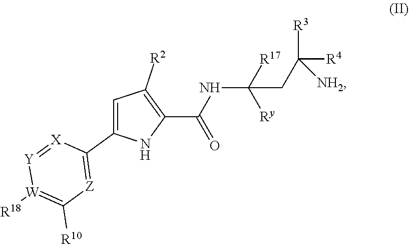
or a pharmaceutically acceptable salt thereof,
wherein:
W is C or N;
X, Y, and Z are independently CH or N, and at least one of W, X, Y, or Z is N; if more than one of W, X, Y, or Z is N, then only X and Y, or X and Z, are both N;
each of R², R³, and R⁴, is, independently, H or CH₃;
R¹⁰ is H when X is N, and is H or OCH₃ when X is CH;
R^{Y} is 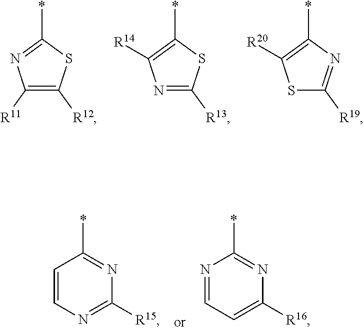
wherein * indicates the point of attachment
Each of R¹¹, R¹², R¹³, R¹⁴, R¹⁵, R¹⁶, R¹⁹, and R²⁰, independently, H, CH₂OH, (CH₂)₂OH, (CH₂)₃OH or CH(OH)CH₂OH;
R¹⁷ is H or CH₃; and
R¹⁸ is absent when W is N, and is H, CH₃, Cl, F, CH₂F, CHF₂, or CF₃ when W is C.

3. The compound of claim 2, wherein the compound is: 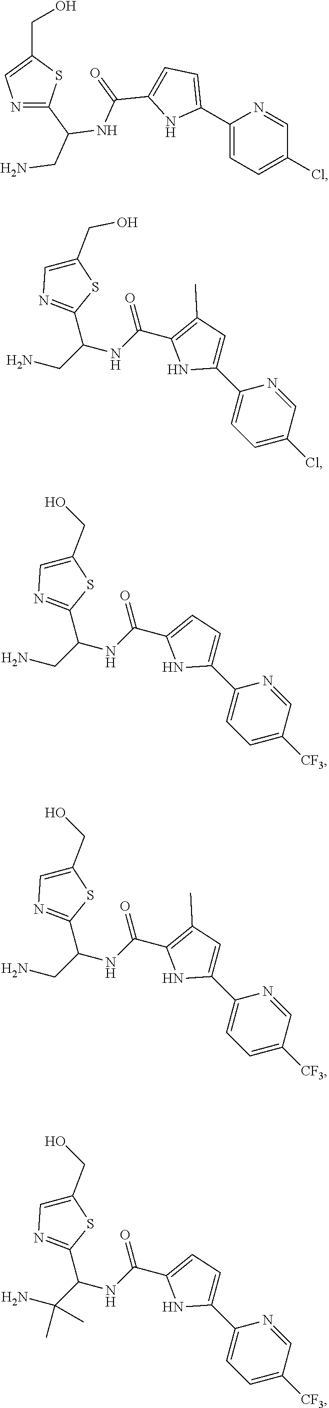 [SMILES: NCC(NC(=O)c1ccc(-c2ccc(Cl)cn2)[nH]1)c1ncc(CO)s1 ; Cc1cc(-c2ccc(Cl)cn2)[nH]c1C(=O)NC(CN)c1ncc(CO)s1 ; NCC(NC(=O)c1ccc(-c2ccc(C(F)(F)F)cn2)[nH]1)c1ncc(CO)s1 ; Cc1cc(-c2ccc(C(F)(F)F)cn2)[nH]c1C(=O)NC(CN)c1ncc(CO)s1 ; CC(C)(N)C(NC(=O)c1ccc(-c2ccc(C(F)(F)F)cn2)[nH]1)c1ncc(CO)s1] 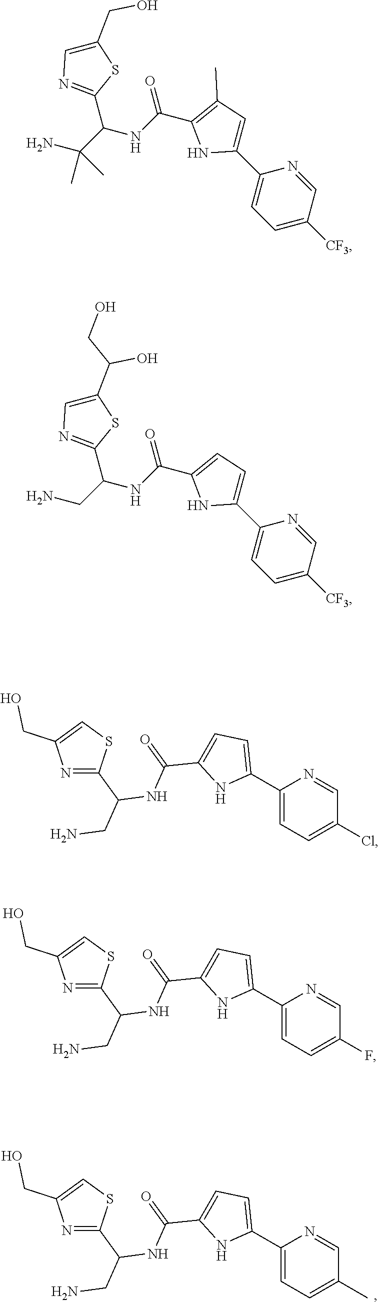 [SMILES: Cc1cc(-c2ccc(C(F)(F)F)cn2)[nH]c1C(=O)NC(c1ncc(CO)s1)C(C)(C)N ; NCC(NC(=O)c1ccc(-c2ccc(C(F)(F)F)cn2)[nH]1)c1ncc(C(O)CO)s1 ; NCC(NC(=O)c1ccc(-c2ccc(Cl)cn2)[nH]1)c1nc(CO)cs1 ; NCC(NC(=O)c1ccc(-c2ccc(F)cn2)[nH]1)c1nc(CO)cs1 ; Cc1ccc(-c2ccc(C(=O)NC(CN)c3nc(CO)cs3)[nH]2)nc1] 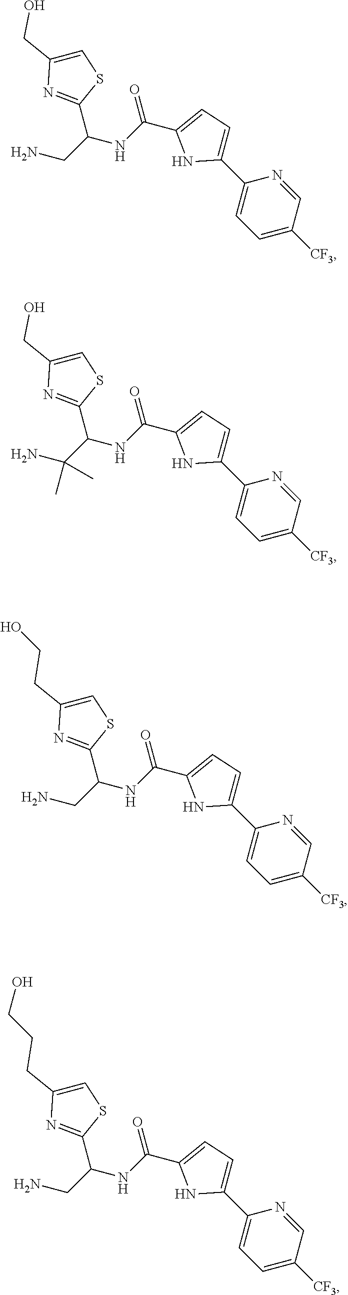 [SMILES: NCC(NC(=O)c1ccc(-c2ccc(C(F)(F)F)cn2)[nH]1)c1nc(CO)cs1 ; CC(C)(N)C(NC(=O)c1ccc(-c2ccc(C(F)(F)F)cn2)[nH]1)c1nc(CO)cs1 ; NCC(NC(=O)c1ccc(-c2ccc(C(F)(F)F)cn2)[nH]1)c1nc(CCO)cs1 ; NCC(NC(=O)c1ccc(-c2ccc(C(F)(F)F)cn2)[nH]1)c1nc(CCCO)cs1] 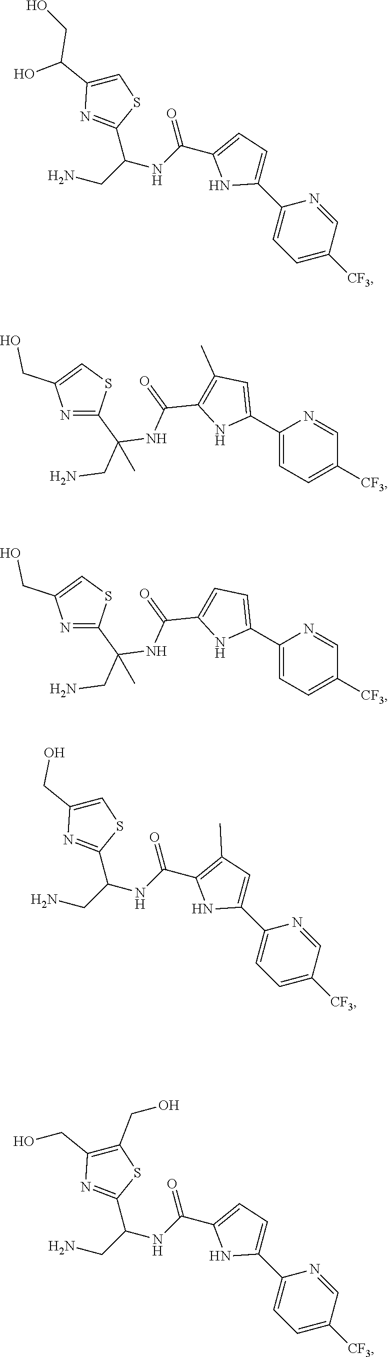 [SMILES: NCC(NC(=O)c1ccc(-c2ccc(C(F)(F)F)cn2)[nH]1)c1nc(C(O)CO)cs1 ; Cc1cc(-c2ccc(C(F)(F)F)cn2)[nH]c1C(=O)NC(C)(CN)c1nc(CO)cs1 ; CC(CN)(NC(=O)c1ccc(-c2ccc(C(F)(F)F)cn2)[nH]1)c1nc(CO)cs1 ; Cc1cc(-c2ccc(C(F)(F)F)cn2)[nH]c1C(=O)NC(CN)c1nc(CO)cs1 ; NCC(NC(=O)c1ccc(-c2ccc(C(F)(F)F)cn2)[nH]1)c1nc(CO)c(CO)s1] 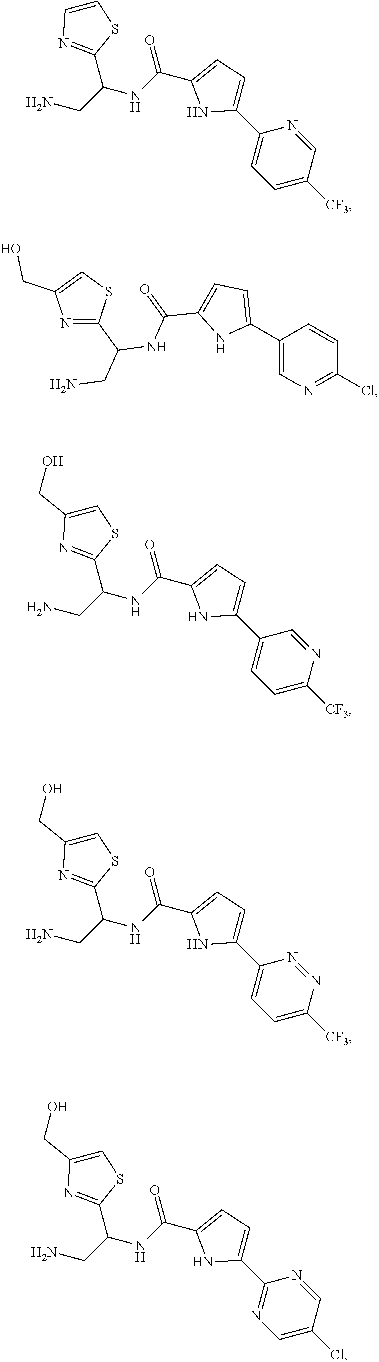 [SMILES: NCC(NC(=O)c1ccc(-c2ccc(C(F)(F)F)cn2)[nH]1)c1nccs1 ; NCC(NC(=O)c1ccc(-c2ccc(Cl)nc2)[nH]1)c1nc(CO)cs1 ; NCC(NC(=O)c1ccc(-c2ccc(C(F)(F)F)nc2)[nH]1)c1nc(CO)cs1 ; NCC(NC(=O)c1ccc(-c2ccc(C(F)(F)F)nn2)[nH]1)c1nc(CO)cs1 ; NCC(NC(=O)c1ccc(-c2ncc(Cl)cn2)[nH]1)c1nc(CO)cs1] 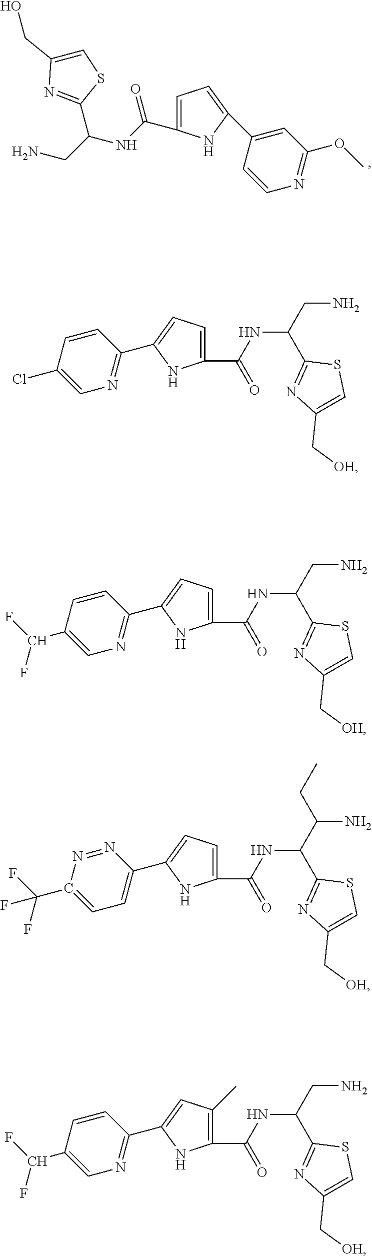 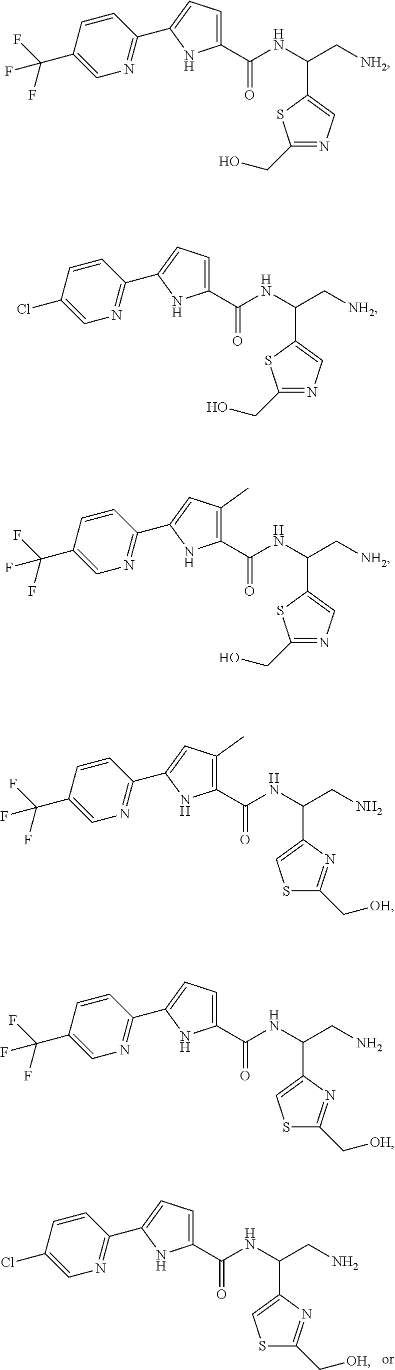 [SMILES: NCC(NC(=O)c1ccc(-c2ccc(C(F)(F)F)cn2)[nH]1)c1cnc(CO)s1 ; NCC(NC(=O)c1ccc(-c2ccc(Cl)cn2)[nH]1)c1cnc(CO)s1 ; Cc1cc(-c2ccc(C(F)(F)F)cn2)[nH]c1C(=O)NC(CN)c1cnc(CO)s1 ; Cc1cc(-c2ccc(C(F)(F)F)cn2)[nH]c1C(=O)NC(CN)c1csc(CO)n1 ; NCC(NC(=O)c1ccc(-c2ccc(C(F)(F)F)cn2)[nH]1)c1csc(CO)n1 ; NCC(NC(=O)c1ccc(-c2ccc(Cl)cn2)[nH]1)c1csc(CO)n1] 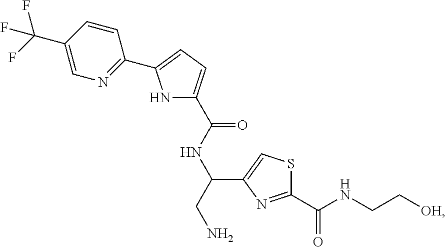 [SMILES: NCC(NC(=O)c1ccc(-c2ccc(C(F)(F)F)cn2)[nH]1)c1csc(C(=O)NCCO)n1]
or a pharmaceutically acceptable salt thereof.

4. (canceled)

5. A pharmaceutical composition, comprising the compound of any one of claims 1-3 and a pharmaceutically acceptable carrier.

6. (canceled)

7. A method of treating an HIV infection in a subject in need thereof, comprising administering to the subject a therapeutically effective amount of a compound of claim 1.

8. The method of claim 7, wherein the HIV infection in the subject is eradicated, reduced, or slowed.

9. The method of claim 7, wherein the HIV viral load in the subject is reduced.

10. The method of claim 7, wherein reoccurrences of the HIV infection in the subject are reduced.

11. The method of claim 7, wherein an adverse physiological impact of the HIV infection in the subject is reduced.

12. The method of claim 7, wherein remission of an organ injury from the HIV infection in the subject is induced.

13. The method of claim 7, wherein the physiological impact of long-term antiviral therapy for the HIV infection in the subject is reduced.

14. The method of claim 6, wherein the inhibition is of a latent HIV infection in the subject.

15. (canceled)
